(19) United States
(12) Patent Application Publication
(10) Pub. No.: US 2023/0280344 A1
(43) Pub. Date: Sep. 7, 2023

(54) Multiple Myeloma Mapping and Uses Thereof

(71) Applicant: The Medical College of Wisconsin, Inc., Milwaukee, WI (US)

(72) Inventors: Jeffrey A. Medin, Shorewood, WI (US); Robyn A. Oldham, Milwaukee, WI (US); Mary L. Faber, Milwaukee, WI (US); Rebekah L. Gundry, Milwaukee, WI (US)

(21) Appl. No.: 18/005,802

(22) PCT Filed: Jul. 15, 2021
(86) PCT No.: PCT/US21/41795

§ 371 Date: Jan. 17, 2023

Related U.S. Application Data
(60) Provisional application No. 63/053,296, filed on Jul. 17, 2020.

(51) Int. Cl. G01N 33/574

(52) U.S. Cl. CPC G01N 33/57407; G01N 2333/70596

(57) ABSTRACT

The present invention provides methods and kits for detecting and treating multiple myeloma. The methods involve detecting proteins that the inventors have identified as biomarkers of multiple melanoma.

### CROSS-REFERENCE TO RELATED APPLICATIONS

This application claims priority to U.S. Provisional Application No. 63/053,296 filed on Jul. 17, 2020, the contents of which are incorporated by reference in its entirety.

### STATEMENT REGARDING FEDERALLY SPONSORED RESEARCH

This invention was made with government support under Grant Numbers R01-HL126785, R01-HL134010, and F31-HL140914 awarded by the National Institutes of Health. The government has certain rights in this invention.

### BACKGROUND

The American Cancer Society estimates that there will be 34,920 new cases and 12,410 deaths from multiple myeloma (MM) in the US in 2021. In MM patients, plasma cell neoplasia can result in bone, nervous system, renal, and hematologic manifestations. Diagnosis of MM is based on morphological features, imaging studies, analysis of myeloma cell-produced proteins, and testing of the blood, urine, and bone marrow (BM). The cell surface antigens CD38 and CD138 can be used to distinguish normal cells from clonal plasma cells, but more extensive use of immunophenotyping has been limited by a lack of universally accepted biomarkers of MM.(3-5) The current standard-of-care for MM includes immunomodulatory drugs (IMiDs), proteasome inhibitors, steroids, and antibody therapies. Initial treatment with bortezomib, lenalidomide, and thalidomide have improved outcomes; however, the majority of MM patients ultimately relapse, necessitating the use of multi-drug combinations.(6) The highly heterogeneous and dynamic nature of MM means that existing therapies are often unable to overcome primary refractory disease and drug-resistant relapses, resulting in a 5-year survival rate of just 50.7%.(7) A comprehensive examination of the MM cell surface is necessary to better define proteins that could be clinically useful for MM diagnosis, stratification, and minimal residual disease tracking.

### SUMMARY

In one aspect, the present disclosure provides methods of detecting multiple myeloma in a sample from a subject having or suspected of having multiple myeloma. The methods comprise detecting the expression of one or more proteins listed in Table 3 at a higher level in the sample than in a non-cancer control.

In a second aspect, the present disclosure provides methods of treating multiple myeloma. The methods comprise detecting the expression of one or more proteins in a sample from a subject having or suspected of having multiple myeloma, and treating the subject with an anti-cancer therapy if at least one of the one or more proteins are detected at a higher level in the sample than in a non-cancer control. The one or more proteins that are detected are selected from the group consisting of: CD5, CD166, CD147, CD98h, CD205, LRBA, CLPTM1L, Homer3, EFNB2, and combinations thereof.

In a third aspect, the present disclosure provides kits for detecting multiple myeloma in a sample from a subject having or suspected of having multiple myeloma. The kits comprise one or more antibodies that are specific to one or more proteins listed in Table 3.

### BRIEF DESCRIPTION OF THE DRAWINGS

FIG. 1 shows an overview of cell surface N-glycoproteins identified by cell surface capture (CSC) analysis of multiple myeloma (MM) and B cell lines. (A) Distribution of protein types identified within each cell line based on UniProt annotations for cluster of differentiation (CD) antigen notations and membrane, single- and multi-pass, GPI- and lipid-anchored proteins. (B) Upset plot(54) showing the distribution of protein observations among B and MM cell lines.

FIG. 2 shows a matrix of CD molecules identified by CSC that were chosen for parallel reaction monitoring (PRM) assay development. For each CD molecule, detection (observed vs. not observed by CSC) in the B cell and MM cell lines in the present study is indicated in the first six columns. Detection by CSC among human cell lines, as described in the cell surface protein atlas (CSPA)(15), is included for comparison. White squares indicate that data are not available for this protein in the CSPA.

FIG. 3 shows the relative abundance of selected proteins assessed by PRM analysis of MM and B cell lines. (A) Syndecan-1 (CD138), (B) ADP-ribosyl cyclase/cyclic ADP-ribose hydrolase 1 (CD38), (C) T-cell surface glycoprotein CD3 delta chain (CD3D), (D) T-cell differentiation antigen CD6, (E) T-cell-specific surface glycoprotein CD28, (F) Neural cell adhesion molecule L1 (L1CAM, CD171), (G) Multimerin-1 (MMRN1), (H) Sortilin (SORT1), (I) Peroxidasin homolog (PXDN), (J) Homer protein homolog 3 (Homer3), (K) B-lymphocyte antigen CD19, and (L) B-lymphocyte antigen CD20 were detectable by PRM in whole cell lysates from the MM and B cell lines. Individual B cell line peak areas are shown in green, and individual MM cell line peak areas are shown in blue. The abundance in a pooled sample control comprising all 6 cell lines is shown in red at the left end of each graph.

FIG. 4 shows a schematic depiction of the target discovery and selection workflow, which indicates the steps completed and number of antigens included at each stage of analysis. MM cell surface proteins were identified by CSC, and proteins of interest were selected. Then, PRM assays were developed, optimized, and used to detect a subset of these proteins of interest in MM patient samples.

FIG. 5 shows the relative abundance of selected proteins detected by PRM analysis of primary human MM patient samples (n=6). (A) Syndecan-1 (CD138), (B) ADP-ribosyl cyclase/cyclic ADP-ribose hydrolase 1 (CD38), (C) CD54 (ICAM1), (D) Integrin B7 (ITGB7), (E) Lipopolysaccharide-responsive and beige-like anchor protein (LRBA), (F) Cleft lip and palate transmembrane protein 1-like protein (CLPTM1L), (G) Homer protein homolog 3 (Homer3), (H) Ephrin-B2 (EFNB2), (I) CD5, (J) Activated leukocyte cell adhesion molecule (ALCAM, CD166), (K) Basigin (CD147), and (L) 4F2 cell-surface antigen heavy chain (CD98hc) all have significantly higher abundance in CD138+ samples compared to in CD138− samples. Individual patient peak areas are shown in blue, where dark shading represents the CD138+ fraction, and light shading represents the CD138− fraction. The average and standard deviation of the six patients per condition are shown at the left in black (CD138+) and white (CD138−). Abundance for a pooled sample control comprising all 6 cell lines is shown in red at the right of each graph. This pooled sample is included only as a control and was not included in statistical comparisons. The mean total fragment ion peak areas of the six patients' CD138+ and CD138− samples were compared using a parametric ratio paired t-test. Statistical significance is assigned by p-value<0.05. On the graphs, p-values are represented by the annotations: n.s. for p>0.05, * for p<0.05, ** for p<0.01, *** for p<0.001, and **** for p<0.0001.

FIG. 6 shows a matrix summarizing the detection of proteins by PRM in MM patient samples. Thirty proteins were detected at significantly higher abundance levels in the MM patient samples compared to their matched controls. For each protein, the gene name and CD annotation are listed. The color gradient in the first 6 columns indicates the relative abundance by PRM within the 6 patient samples (dark blue=highest abundance, light blue=lowest abundance). The levels detected by CSC among human cell lines, as described in the CSPA(15), is included for comparison (black=detected, white=not detected; grey=no data available).

FIG. 7 shows a validation of selected proteins detected by PRM in a second cohort of primary MM patient samples. (A) CD166, (B) CD147, (C) CD98hc, (D), CD205, (E) LRBA, (F) CLPTML1, (G) Homer 3, (H) EFNB2. Individual patient peak areas are shown in blue, where dark shading represents the CD138+ fraction and light shading represents the CD138− fraction. The average and standard deviation of the 4 patients per condition is shown at the left in black (CD138+) and white (CD138−). Abundance for a pooled sample control comprising all 6 cell lines is shown in red at the right of each graph. This pooled sample is included only as a control and was not included in statistical comparisons. The mean total fragment ion peak areas of the six patients' CD138+ and CD138− samples were compared using a parametric ratio paired t-test. Statistical significance is assigned by p-value<0.05. On the graphs, p-value is represented by the annotations: n.s. for p>0.05, * for p<0.05, and ** for p<0.01.

FIG. 8 shows a flow cytometric analyses of target antigen expression on primary MM samples. Expression on CD138+MM cells (blue) and CD138− bone marrow (BM) cells (green) from matched patient samples is shown for the 5 candidate antigens: (A) CD98hc, (B) CD166, (C) CD147, (D) CD205, and (E) CD5. CD138+ and CD138− isolated BM cells from 4 patients were available for flow cytometry (FCM) analysis. The x-axis shows log¹⁰ fluorescence intensities for each antibody, while the y-axis shows cell counts normalized to maximum of cells collected for each sample (20,000 cells per sample). Staining with antibody is shown as open histograms, and isotype staining is shown as shaded histograms.

FIG. 9 shows a matrix of non-CD proteins that were chosen for PRM assay development. For each protein, detection (observed vs. not observed by CSC) in the B cell and MM cell lines in the present study is indicated in the first six columns. Detection by CSC among human cell lines, as described in the CSPA (55), is included for comparison. White squares indicate that data are not available for this protein in the CSPA.

FIG. 10 shows the relative abundance of proteins detected by PRM analysis of whole cell lysates of MM and B cell lines. (A) Kunitz-type protease inhibitor 1 (SPINT1), (B) Calumenin (CALU), (C) Adenylate cyclase type 3 (ADCY3), (D) CD205 (LY75), (E) Protein CREG1 (CREG1), (F) Prothrombin (F2), (G) Metalloproteinase inhibitor 1 (TIMP1), (H) Immunoglobulin heavy constant gamma 2 (IGHG2), (I) Transferrin receptor protein 1 (TFR1), (J) Serotransferrin (TF), (K) Phosphatidylcholine-sterol acyltransferase (LCAT), (L) Thy-1 membrane glycoprotein (CD90), (M) Sodium/potassium-transporting ATPase subunit alpha-1 (ATP1A1), (N) Sodium/potassium-transporting ATPase subunit beta-1 (ATP1B1), (O) Integrin B2 (ITGB2), (P) Intercellular adhesion molecule 1 (ICAM1) (Q) T-cell surface glycoprotein CD5 (R) 4F2 cell-surface antigen heavy chain (CD98hc) (S) CD45 (PTPRC), (T) Leukocyte antigen CD37, (U) Lysosomal-associated membrane protein 1 (LAMP1, CD107a), (V) Coagulation factor V (F5), (W) Intercellular adhesion molecule 2 (ICAM2), (X) CD44, (Y) Sarcoplasmic/endoplasmic reticulum calcium ATPase 2 (SERCA2), (Z) Plasma membrane calcium-transporting ATPase 1 (PMCA1), (AA) TATA-box-binding protein (TBP), (AB) Voltage-dependent anion-selective channel protein 1 (VDAC1), (AC) Plasma membrane calcium-transporting ATPase 4 (ATP2B4), (AD) Integrin beta-7 (ITGB7) (AE) Intercellular adhesion molecule 3 (ICAM3), (AF) Multidrug resistance-associated protein 1 (ABCC1), (AG) Basigin (CD147), (AH) Receptor-type tyrosine-protein kinase FLT3 (FLT3), (AI) Cell surface glycoprotein MUC18 (MCAM), (AJ) Translocon-associated protein subunit alpha (SSR1), (AK) Lipopolysaccharide-responsive and beige-like anchor protein (LRBA), (AL) Ephrin-B2 (EFNB2) (AM) Sodium/potassium-transporting ATPase subunit beta-3 (ATP1B3), (AN) Microfibril-associated glycoprotein 4 (MFAP4), (AO) Histone H3.3 (H3F3A), (AP) Transcription factor A, mitochondrial (TFAM), (AQ) Tumor necrosis factor receptor superfamily member 17 (BCMA), (AR) Acid ceramidase (ASAH1), (AS) CD166 (ALCAM), (AT) P-selectin glycoprotein ligand 1 (SELPLG), (AU)C-type lectin domain family 12 member A (CLEC12A), (AV) Osteoclast-associated immunoglobulin-like receptor (OSCAR), (AW) Prenylcysteine oxidase-like (PCYOXIL), (AX) SLAM family member 6 (SLAMF6), (AY) Ceramide synthase 2 (CERS2), (AZ) Cleft lip and palate transmembrane protein 1-like protein (CLPTMIL), (BA) Protein tweety homolog 2 (TTYH2), (BB) Probable cation-transporting ATPase 13A3 (ATP13A3), (BC) Transmembrane protein 206 (TMEM206), (BD) Ceramide synthase 4 (CERS4), (BE) Adenosine deaminase 2 (ADA2), (BF) Calmodulin-like protein 5 (CALML5), (BG) Peroxisome proliferator-activated receptor gamma coactivator 1-alpha (PPARGCIA), (BH) N-acetylglucosamine-1-phosphotransferase subunit gamma (GNPTG), and (BI) Bis(5′-adenosyl)-triphosphatase ENPP4 (ENPP4) were detectable by PRM in whole cell lysates from the B and MM cell lines. Individual B cell line peak areas are shown in green, and individual MM cell line peak areas are shown in blue. Abundance for a pooled sample control comprising all 6 cell lines is shown in red at the left of each graph.

FIG. 11 shows the relative abundance of proteins detected by PRM analysis of whole cell lysates of primary human MM patient samples. (A) Calumenin (CALU), (B) Adenylate cyclase type 3 (ADCY3), (C) CD205 (LY75), (D) Prothrombin (F2), (E) Metalloproteinase inhibitor 1 (TIMP1), (F) Immunoglobulin heavy constant gamma 2 (IGHG2), (G) Transferrin receptor protein 1 (TFR1), (H) Serotransferrin (TF), (I) Phosphatidylcholine-sterol acyltransferase (LCAT), (J) Thy-1 membrane glycoprotein (CD90), (K) CD3 delta (CD3D), (L) Sodium/potassium-transporting ATPase subunit alpha-1 (ATP1A1), (M) Sodium/potassium-transporting ATPase subunit beta-1 (ATP1B1), (N) Integrin B2 (ITGB2), (O) CD45 (PTPRC), (P) CD37, (Q) Lysosomal-associated membrane protein 1 (LAMP1, CD107a), (R) CD20, (S) Coagulation factor V (F5), (T) Intercellular adhesion molecule 2 (ICAM2), (U) CD44, (V) Sarcoplasmic/endoplasmic reticulum calcium ATPase 2 (SERCA2), (W) Plasma membrane calcium-transporting ATPase 1 (PMCA1), (X) TATA-box-binding protein (TBP), (Y) Voltage-dependent anion-selective channel protein 1 (VDACI), (Z) Plasma membrane calcium-transporting ATPase 4 (ATP2B4), (AA) Intercellular adhesion molecule 3 (ICAM3), (AB) Translocon-associated protein subunit alpha (SSR1), (AC) Sodium/potassium-transporting ATPase subunit beta-3 (ATP1B3), (AD) Microfibril-associated glycoprotein 4 (MFAP4), (AE) Histone H3.3 (H3F3A), (AF) Transcription factor A, mitochondrial (TFAM), (AG) Tumor necrosis factor receptor superfamily member 17 (BCMA), (AH) Acid ceramidase (ASAH1), (AI) P-selectin glycoprotein ligand 1 (SELPLG), (AJ) Osteoclast-associated immunoglobulin-like receptor (OSCAR), (AK) Prenylcysteine oxidase-like (PCYOXIL), (AL) Peroxidasin homolog (PXDN), (AM) Ceramide synthase 2 (CERS2), (AN) Sortilin (SORT1), (AO) Protein tweety homolog 2 (TTYH2), (AP) Transmembrane protein 206 (TMEM206), (AQ) Adenosine deaminase 2 (ADA2), (AR) Calmodulin-like protein 5 (CALML5), (AS) Peroxisome proliferator-activated receptor gamma coactivator 1-alpha (PPARGCIA), (AT)N-acetylglucosamine-1-phosphotransferase subunit gamma (GNPTG), and (AU) Bis(5′-adenosyl)-triphosphatase ENPP4 (ENPP4) were detectable by PRM in the patient samples. Individual patient peak areas are shown in blue, where dark shading represents the CD138+ fraction and light shading represents the CD138− fraction. The average and standard deviation of the 6 patients per condition is shown at the left in black (CD138+) and white (CD138−). Abundance for a pooled sample control comprising all 6 cell lines is shown in red at the right of each graph. This pooled sample is included only as a control and was not included in statistical comparisons. The mean total fragment ion peak areas of the six patients' CD138+ and CD138− samples were compared using a parametric ratio paired t-test. Statistical significance is assigned by p-value<0.05. On the graphs, p-value is represented by the annotations: n.s. for p>0.05, * for p<0.05, ** for p<0.01, *** for p<0.001, and **** for p<0.0001.

FIG. 12 shows a flow cytometric analysis of target antigen expression in normal hematopoietic cells. Expression of the 5 candidate antigens was detected in (A) whole normal BM cells, (B) freshly purified peripheral blood CD19+ normal B cells, and (C) freshly purified peripheral blood CD3+ normal T cells from healthy donors. The x-axis shows log₁₀ fluorescence intensities for each antibody, while the y-axis shows cell counts normalized to the maximum of cells collected for each sample (20,000 cells per sample). Staining with antibody is shown as open histograms and isotype staining is shown as shaded histograms.

### DETAILED DESCRIPTION

The present inventors have identified novel proteins that can be used as biomarkers of multiple melanoma (MM). The inventors first identified N-glycoproteins present on the surface of MM cells using cell surface capture (CSC), and then and used a set of novel parallel reaction monitoring (PRM) assays to identify the subset of the glycoproteins that are significantly more abundant in MM patient cells than in control cells. Finally, the inventors selected nine of the MM-enriched proteins as potential immunotherapeutic targets, and used flow cytometry to confirm that these proteins are expressed on the cell surface of primary MM patient cells. Accordingly, the present disclosure provides methods and kits for detecting multiple myeloma.

### Methods:

In a first aspect, the present invention provides methods of detecting multiple myeloma in a sample from a subject having or suspected of having multiple myeloma. The methods comprise detecting the expression of one or more proteins listed in Table 3 at a higher level in the sample than in a non-cancer control.

Multiple myeloma (MM) is a cancer of plasma cells, a type of white blood cell that produces antibodies. It is the second most common hematological cancer. MM is characterized by the accumulation of neoplastic plasma cells in the bone marrow, which can result in osteolytic lesions, anemia, renal failure, and hypercalcemia. Symptoms of MM include bone pain, bleeding, and frequent infections.

In Table 3, the inventors disclose 30 proteins that are putative biomarkers of MM. As used herein, the term “biomarker” or “marker” refers to a detectable molecule that is differentially expressed in a particular condition. The expression of a biomarker is correlated with a condition such that measuring its concentration (e.g., expression level) may be useful for predicting, prognosticating, or diagnosing the condition. The 30 proteins listed in Table 3 were detected at significantly higher levels in CD138+ cells isolated from bone marrow samples of MM patients as compared to in CD138− cells isolated from the same samples. CD138 (also known as syndecan-1) is a member of the syndecan family of transmembrane heparan sulfate proteoglycans, and expression of CD138 is a hallmark of plasma cells and multiple myeloma cells. Thus, CD138+ cells isolated from the bone marrow of MM patients are enriched for MM cancer cells.

As used herein, the terms “protein” and “polypeptide” are used interchangeably to designate a series of amino acid residues connected by peptide bonds between the alpha-amino and carboxy groups of adjacent residues. The terms “protein” and “polypeptide” refer to a polymer of protein amino acids, including modified amino acids (e.g., phosphorylated, glycated, glycosylated, etc.) and amino acid analogs. “Protein” and “polypeptide” are often used in reference to relatively large polypeptides, whereas the term “peptide” is often used in reference to small polypeptides, but usage of these terms in the art overlaps.

The inventors identified the proteins disclosed in Table 3 in a screen for extracellular glycoproteins that are found on the surface of MM cells. Glycoproteins are proteins that comprise oligosaccharide chains that are covalently attached to amino acid side-chains. The oligosaccharide chains are added to most secreted extracellular proteins as a co-translational or posttranslational modification through a process is known as glycosylation. Thus, enriching for glycosylated proteins allowed the inventors to assay a large subset of the cell surface proteome. Using cell surface proteins, as opposed to intracellular proteins, as biomarkers of MM offers the added benefit that the same proteins can be used as immunotherapy targets. Cell surface proteins are also ideal targets for the development of flow cytometry-based diagnostic/prognostic assays.

In some embodiments, several of these proteins are used in combination as biomarkers of MM. Advantageously, one might detect the levels of one, two, three, four, five, six, seven, eight, nine, or more of the proteins disclosed in Table 3 in the present methods. For instance, in some embodiments, the methods comprise detecting three or more proteins listed in Table 3. In some embodiments, the methods comprise detecting five or more proteins listed in Table 3. In some embodiments, the methods comprise detecting nine or more proteins listed in Table 3.

The inventors selected 9 of the 30 protein biomarkers as potential therapeutic targets for MM. The 9 potential therapeutic targets include 5 proteins (i.e., CD5, CD166, CD147, CD98hc, and CD205) that are currently under investigation as therapeutic targets for other cancer types, and 4 proteins (i.e., LRBA, CLPTM1L, Homer3, and EFNB2) that have not been previously described as therapeutic targets for any malignancy. Thus, in some embodiments, the one or more proteins detected comprise at least one of the nine potential therapeutic targets (i.e., CD5, CD166, CD147, CD98h, CD205, LRBA, CLPTM1L, Homer3, EFNB2, and combinations thereof). Alternatively, the one or more proteins detected may comprise at least two of the nine, at least three of the nine, at least four of the nine, at least five of the nine, at least six of the nine, at least seven of the nine, at least eight of the nine, or all nine of the potential therapeutic targets, i.e., CD5, CD166, CD147, CD98h, CD205, LRBA, CLPTM1L, Homer3, and EFNB2. In some certain embodiments, the one or more proteins detected comprise at least one of the four potential therapeutic targets that was not previously reported (i.e., LRBA, CLPTM1L, Homer3, EFNB2, and combinations thereof). Alternatively, the one or more proteins detected may comprise at least two of the four, at least three of the four, or all four of the proteins LRBA, CLPTM1L, Homer3, and EFNB2.

In the Examples, the inventors confirmed that a subset of the 9 potential therapeutic targets (i.e., CD5, CD166, CD147, CD98hc, and CD205) are expressed on the surface of CD138+MM patient cells using flow cytometry. Notably, the remaining four potential therapeutic targets (i.e., LRBA, CLPTM1L, Homer3, EFNB2) were not be assessed using flow cytometry because suitable antibodies to these proteins were not available. Thus, in some embodiments, the one or more proteins are detected via flow cytometry. In particular embodiments, one or more proteins selected from CD5, CD166, CD147, CD98hc, CD205, and combinations thereof are detected via flow cytometry.

In some embodiments, the methods further comprise treating the subject with an anti-cancer therapy. As used herein, an “anti-cancer therapy” is a therapy that is administered to treat a cancer. In some embodiments, the anti-cancer therapy is specific for MM, meaning (1) that it has been formulated to treat this particular type of cancer or (2) that it has been shown to effectively treat MM. Suitable anti-cancer therapies for the treatment of MM include, without limitation, immunomodulatory drugs (e.g., thalidomide (Thalomid), bortezomib (Velcade), lenalidomide (Revlimid), and pomalidomide (Pomalyst)), proteasome inhibitors, chemotherapies, corticosteroids (e.g., prednisone and dexamethasone), radiation therapy, antibody therapies, chimeric antigen receptor (CAR) therapies, and bone marrow transplant. Often, these therapies are administered in combination. For example, prior to a bone marrow transplant, patients are given high doses of chemotherapy to destroy diseased bone marrow. Thus, in some embodiments, the methods further comprise treating the subject with a combination of anti-cancer therapies.

In some embodiments, the anti-cancer therapy is a targeted therapy, i.e., a therapy that interferes with a molecule involved in the growth, progression, and/or spread of the cancer. In many targeted therapies, antibodies that specifically bind to a protein present on the cancer cell surface (e.g., the biomarkers disclosed herein) are used to target and kill these cells. Suitable antibody-based targeted therapies include, without limitation, those comprising antibodies, antibody-drug conjugates, bispecific antibodies, and radioimmunotherapies. For example, some MM drugs comprise an antibody that simply binds to cancer cells, “flagging” them to help the immune system identify and attack them (e.g., elotuzumab (Empliciti™, a monoclonal antibody to CS1, expressed on MM cells)). In other MM drugs, an antibody that targets an MM biomarker is conjugated to a cytotoxic drug or radioactive material that kills the cells. Other targeted therapies utilize chimeric antigen receptor T cells (CAR T cells), i.e., T cells that have been genetically engineered to express an artificial T-cell receptor that recognizes an MM biomarker, thereby targeting the modified T cells to destroy cancer cells. Often, CAR T cells are engineered to express chimeric receptors that have an antigen recognition domain that is derived from the variable regions of an antibody. However, any protein or peptide that targets an appropriate biomarker may be used to in the antigen recognition domain to endow the CAR T cell with the desired specificity.

Using the same biomarker to target/treat MM that was used to diagnose MM may offer advantages in terms of both efficiency and efficacy. Thus, in certain preferred embodiments, the anti-cancer therapy used with the present invention specifically targets the protein that was detected in the sample from the subject in the first step of the method. At least five of the disclosed protein biomarkers (i.e., CD5, CD147, CD205, CD98, and CD166) are targets of anti-cancer therapies that are being developed for use against various cancers. Thus, if one or more of these biomarkers are detected, the corresponding anti-cancer therapy can be used to specifically target cancer cells expressing the biomarker(s) to treat the subject.

For example, if the detected biomarker is CD5, an anti-CD5 anti-cancer therapy may be used to treat the subject. The term “anti-CD5” indicates that the anti-cancer therapy is specifically targeted to cancer cells that express CD5. Likewise, an anti-cancer therapy that targets biomarker “X” can be referred to as an “anti-X anti-cancer therapy”. Suitable anti-CD5 anti-cancer therapies include, for example, anti-CD5 chimeric antigen receptor T (CAR-T) cells. CD5 targeting CAR-T cells have been tested in clinical trials (e.g., NCT03081910) and are described in the literature. See, e.g., Blood (2015) 126(8):983-92, which is incorporated by reference in its entirety regarding CAR-T cells.

In another embodiment, the detected biomarker is CD147, and an anti-CD147 anti-cancer therapy is used to treat the subject. Suitable anti-CD147 anti-cancer therapies, including antibodies and small molecules specific for CD147, are known in the art and are under development. For example, several anti-CD147 therapies are described by Landras et al. (Cancers (Basel) (2019) 11(11):1803) including the antibody MEM-M6/1, Acriflavine (ACF), the antibody 161-Ab, the antibody 059-053, and the antibody CNTO3899. Other anti-CD147 therapies that have been described in the literature include the antibodies 1B3 and 3B3 described by Wang et al. (Hybridoma (Larchmt) (2006) 25:60-67); the agent Licartin (generic name: (I¹³¹) metuximab), which comprises the anti-CD147 monoclonal antibody HAb18 conjugated to the radioisotope I¹³¹; alternate forms HAb18, including a chimeric antibody; the cHAb18 antibody described by Chen et al. (EP Patent No. 20030711796), which contains the variable heavy and light chains of the antibody HAb18 and the constant regions of human IgG1γ1; and the HcHAb18 antibody conjugates described by Huhe et al. (Biochem Biophys Res Commun (2019) 513:1083-1091), which are conjugated to cytotoxic drugs. Each of the above references (i.e., Landras et al., Wang et al., Chen et al., Huhe et al.) are hereby incorporated by reference in their entirety with regards to CD147-based therapies.

In another embodiment, the detected biomarker is CD205, and an anti-CD205 anti-cancer therapy is used to treat the subject. Suitable anti-CD205 anti-cancer therapies are known in the art and include, for example, the anti-CD205 therapies described in Merlino et al. (Molecular Cancer Therapeutics (2019) 18(9):1533-1543); Gaudio et al. (Hematologica (2020) 105(11):2584-2591); and Canzonieri et al. (Journal of Clinical Oncology (2017) 35(15_suppl)), each of which is incorporated by reference in their entirety with regards to CD205-based therapies.

In another embodiment, the detected biomarker is CD98, and an anti-CD98 anti-cancer therapy is used to treat the subject, preferably an antibody against CD98. Suitable anti-CD98 antibodies are known in the art and include those described in Bixby et al. (Blood (2015) 126 (23):3809); Hayes et al. (International Journal of Cancer (2015) 137(3):710-720); and Bajaj et al. (Cancer Cell (2016) 30(5):792-805), each of which is incorporated by reference in its entirety regarding anti-CD98 antibodies.

In another embodiment, the detected biomarker is CD166, and an anti-CD166 anti-cancer therapy is used to treat the subject, preferably an antibody-drug conjugate targeting CD166. Suitable anti-CD166 antibodies are known in the art and include, for example, those described in Boni et al. (J Clin Oncol 38: 2020 (suppl; abstr 526)); Wiiger et al. (Cancer Immunology, Immunotherapy (2010) 59(11):1665-1674); and Roth et al. (Molecular Cancer Therapeutics (2007) 6(10):2737-2746), the contents of which are incorporated by reference in their entireties regarding anti-CD166 therapies.

The anti-cancer therapies used with the present invention may be administered by any suitable method. However, those of skill in the art understand that the method of administration must be selected with both the particular anti-cancer therapy and the subject being treated in mind. Suitable methods of administration include, for example, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, intradermal administration, intrathecal administration, and subcutaneous administration. Administration of the anti-cancer therapies can be continuous or intermittent.

As used herein, the term “subject” refers to either a human or non-human animal. Examples of non-human animals include vertebrates, such as non-human primates, dogs, rodents (e.g., mice, rats, or guinea pigs), pigs and cats, etc. In a preferred embodiment, the subject is a human patient having or suspected of having MM. A subject may be suspected of having MM, for example, if the subject exhibits a symptom of MM or if the results of a diagnostic test are suggestive of MM. Symptoms of MM include, without limitation, bone pain, nausea, constipation, loss of appetite, mental fogginess or confusion, fatigue, frequent infections, weight loss, weakness or numbness in the legs, and excessive thirst. Diagnostic tests for MM include, for example, blood and urine tests (e.g., those that measure M protein, M protein light chain, immunoglobulin levels, serum albumin, or serum beta-2 microglobulin), x-rays, magnetic resonance imaging, computed tomography (CAT) scan, positron emission tomography (PET) scan, bone marrow biopsies, fat pad aspirates, or molecular testing of a tumor (e.g., to identify genes or proteins associated with MM).

As used herein, the term “sample” refers to a sample derived from a subject having or suspected of having multiple myeloma. The sample may be obtained directly from the subject or may be derived from cultured cells obtained from the subject. Suitable samples include, without limitation, bone marrow aspirates, bone marrow biopsies, lymph node samples, urine samples, and blood samples. In some embodiments, the sample is a biopsy from the subject. In some embodiments, the sample is a peripheral blood sample. In certain preferred embodiments, the sample is a bone marrow sample, and the methods comprise obtaining a bone marrow sample from a subject. Methods of obtaining a bone marrow sample are known in the art. Such methods commonly involve removing bone marrow through a small, hollow needle that is inserted into the bone of the subject.

In some embodiments, to enrich for MM cells, the methods further comprise isolating CD138+ cells from the sample prior to detecting the one or more proteins. CD138+ cells may be isolated using an anti-CD138 antibody, e.g., in an antibody pull-down assay, affinity purification, or fluorescence-activated cell sorting (FACS). In certain embodiments, the CD138+ cells are isolated using flow cytometry or magnetic beads (e.g., Whole Blood CD138 MicroBeads from Miltenyi Biotec). However, enriching for MM cells may not be necessary, especially for patients with a high disease burden. Thus, in other embodiments, the sample is not enriched for CD138+ cells prior to use.

The methods of the present invention involve detection of biomarkers that the inventors have identified as having increased expression on MM cancer cells. While the disclosed protein biomarkers were identified using proteomics, upregulation of these biomarkers may be detected at either the protein or RNA level in the present methods. RNA detection can be performed by any suitable method including, for example, reverse transcription polymerase chain reaction (RT-PCR), quantitative PCR, nuclear run-on assays, RNase protection assays, Northern blotting, in situ hybridization, microarray analysis, or any RNA sequencing method. To facilitate detection, the RNA may first be isolated from cells using an RNA extraction technique, such as guanidinium thiocyanate-phenol-chloroform extraction (e.g., using TRIzol), trichloroacetic acid/acetone precipitation followed by phenol extraction, or using commercially available column-based system (e.g., RNeasy RNA Preparation Kit from Qiagen). Such techniques are well known in the art.

In preferred embodiments, the biomarkers are detected at the protein level. Protein detection can be performed by any suitable method including, for example, immunoassays, flow cytometry, mass spectrometry, western blot, and 2-D PAGE. In the Examples, the inventors used flow cytometry to confirm that a subset of the disclosed protein biomarkers are expressed on the surface of patient-derived MM cells. Flow cytometry is a widely used method for analyzing cell surface protein expression. In this method, a sample containing cells is suspended in a fluid and injected into the flow cytometer instrument, which focuses the flow of a sample such that roughly one cell passes through a laser beam at a time. Proteins of interest can be labeled on the cell with a fluorescent marker (e.g., via a fluorescently labeled antibody) prior to flow cytometry, such that light is absorbed and then emitted at a particular wavelength if a cell comprises the labeled protein. With this method, tens of thousands of cells can be quickly examined. Thus, in certain embodiments, the one or more proteins are detected using flow cytometry.

The inventors initially identified the disclosed protein biomarkers using a mass spectrometry-based proteomics method. As used herein, the term “mass spectrometry-based method” refers to any method that utilizes mass spectrometry, an analytical technique that measures the mass-to-charge ratio of ions. The mass spectrometry-based method that the inventors utilized to identify the biomarkers is known as cell surface capture (CSC). In CSC, cell surface oligosaccharides are labeled and captured to enrich for extracellular glycoproteins in a sample before it is applied to a mass spectrometer. Thus, in some embodiments, the one or more protein is detected using a mass spectrometry-based method. In particular embodiments, the mass spectrometry-based method is cell surface capture (CSC).

The inventors developed a series of parallel reaction monitoring (PRM) assays that can be used to detect a subset of the identified biomarker proteins. PRM is an ion monitoring technique that uses a high-resolution hybrid mass spectrometer, such as a Q-Orbitrap. PRM is suitable for the quantification of multiple proteins in complex samples at an attomole-level of detection. PRM first uses a quadrupole to select a precursor ion, then the precursor ion is fragmented in the collision cell, and all product ions are scanned with high resolution and accuracy. Following data acquisition, quantification is carried out using selected fragment ions. A more detailed description PRM can be found in the art, for example, the description by Rauniyar (Int. J. Mol. Sci. (2015) 16:28566-28581), the contents of which are incorporated by reference in their entirety. Importantly, PRM allows for targeted, quantitative detection of a particular biomarker using far fewer cells than are required for CSC (less than 1 million vs. over 50 million). Thus, in preferred embodiments, the mass spectrometry-based method is a PRM assay.

In the present methods, the level of a biomarker detected in the sample is compared to the level detected in a non-cancer control. As used herein, the term “non-cancer control” refers to a sample comprising noncancerous cells. For example, the non-cancer control may comprise cell from a healthy subject, i.e., a subject that exhibits no signs of MM. The non-cancer control may also comprise a noncancerous cell line or cells remaining from a patient sample after any malignant cells have been removed. Alternatively, the non-cancer control may be a reference sample, i.e., a sample in which the level of the biomarker has been established, allowing it to serve as a standard. The non-cancer control may comprise CD138− cells and/or CD138+ cells (e.g., from the bone marrow of a healthy subject). The non-cancer control may comprise plasma cells or cells of another cell type, such as B cells.

In a second aspect, the present invention provides methods of treating multiple myeloma. The methods comprise (1) detecting the expression of one or more proteins in a sample from a subject having or suspected of having multiple myeloma, and (2) treating the subject with an anti-cancer therapy if at least one of the one or more MM biomarker proteins is detected at a higher level in the sample than in a non-cancer control. For these methods, the one or more proteins are selected from the biomarkers listed in Table 3. Preferably, the one or more proteins include at least one or more of the nine potential therapeutic targets identified by the inventors, namely CD5, CD166, CD147, CD98h, CD205, LRBA, CLPTM1L, Homer3, and EFNB2.

As used herein, “treating” or “treatment” describes the management and care of a subject for the purpose of combating a disease, condition, or disorder. Treating includes administering a treatment to prevent the onset of the symptoms or complications, to alleviate the symptoms or complications, or to eliminate the disease, condition, or disorder. For example, treating cancer in a subject includes the reducing, repressing, delaying or preventing of cancer growth, reduction of tumor volume, and/or preventing, repressing, delaying or reducing metastasis of the tumor. Treating cancer in a subject also includes the reduction of the number of tumor cells within the subject.

In some embodiments, the anti-cancer therapy used with these methods is a therapy that specifically targets the protein detected in the sample (i.e., in the subject's cells). Specifically, in some embodiments, the methods comprise one or more of the following steps: (a) detecting increased expression of CD5 in the sample, and treating the subject with an anti-CD5 anti-cancer therapy, preferably a CD5 chimeric antigen receptor T cell; (b) detecting increased expression of CD147 in the sample, and treating the subject with an anti-CD147 anti-cancer therapy, preferably a radioimmunotherapy; (c) detecting increased expression of CD205 in the sample, and treating the subject with an anti-CD205 anti-cancer therapy, preferably an anti-CD205 antibody-drug conjugate; (d) detecting increased expression of CD98 in the sample, and treating the subject with an anti-CD98 anti-cancer therapy, preferably an antibody against CD98; and/or (e) detecting increased expression of CD166 in the sample, and treating the subject with an anti-CD166 anti-cancer treatment, preferably an antibody-drug conjugate targeting CD166.

### Kits:

In a third aspect, the present invention provides kits for detecting multiple myeloma in a sample from a subject having or suspected of having multiple myeloma. The kits comprise one or more antibodies that are specific to one or more of the protein biomarkers listed in Table 3. An antibody is “specific” to a protein if it binds to that protein in preference to other molecules. A specific antibody does not bind to molecules other than the target protein in a significant amount. Specific binding can also mean that the antibody binds to the target protein with an affinity that is at least 25% greater, at least 50% greater, at least 100% (2-fold) greater, at least ten times greater, at least 20-times greater, or at least 100-times greater than the affinity with which it binds to any other molecule.

The antibodies included in the kits enable detection of the protein biomarkers disclosed herein using an antibody-based detection method. Suitable antibody-based detection methods include, for example, western blot, flow cytometry, and enzyme-linked immunosorbent assay (ELISA). Notably, an ELISA may be performed either on whole-cells or on cell lysates using the kits of the present invention.

In some embodiments, a panel of antibodies is used in an ELISA, wherein each antibody in the panel targets a different biomarker listed in Table 3. Suitable antibody panels may include a solid support to which the antibodies are attached, for example, a plate, a filter, a plastic surface, a microtiter plate, a tissue culture plate, a tube or the like. In one example, the antibodies may be specific to one or more of the nine potential therapeutic targets identified by the inventors (i.e., CD5, CD166, CD147, CD98h, CD205, LRBA, CLPTM1L, Homer3, and EFNB2). Alternatively, the antibodies may be specific to two or more, three or more, four or more, five or more, six or more, seven or more, eight or more, or all nine of the protein biomarkers selected from CD5, CD166, CD147, CD98h, CD205, LRBA, CLPTM1L, Homer3, and EFNB2. In another example, the kit or panel comprises antibodies that are specific to one or more, two or more, three or more, four or more, or all five of the protein biomarkers selected from CD138, CD38−, CD45, CD19, and CD56.

Suitable samples for use with the present kits include, without limitation, bone marrow and peripheral blood. Advantageously, mononuclear cells are isolated from such samples before protein detection is performed. Thus, in some embodiments, the kits further comprise a reagent for isolating mononuclear cells. Suitable reagents for isolating mononuclear cells include, for example, Ficoll, cell preparation tubes (CPTs), and SepMate™ tubes (Stemcell Technologies).

To facilitate biomarker detection in a sample comprising a low percentage of MM cells, it may be advantageous to enrich the sample for CD138+ cells prior to protein detection. Thus, in some embodiments, the kits further comprise a reagent for isolating CD138+ cells. Such reagents may include, for example, an anti-CD138 antibody that can be used to isolate CD138+ cells from the sample, e.g., using an antibody pull-down assay, affinity purification, or flow cytometry.

In some embodiments, the kits further comprise at least one additional reagent selected from the group consisting of: a lysis buffer, non-cancer control cells (i.e., to be used as a negative control), and instructions for using the kit.

### Examples

Multiple myeloma (MM) is characterized by clonal expansion of malignant plasma cells in the bone marrow. While recent advances in treatment for MM have improved patient outcomes, the 5-year survival rate remains ˜50%. A better understanding of the MM cell surface proteome could facilitate development of new directed therapies and assist in stratification and monitoring of patient outcomes.

In the following Example, the inventors first used a mass spectrometry (MS)-based discovery-driven cell surface capture (CSC) approach to map the cell surface N-glycoproteome of MM cell lines. They then developed targeted MS assays and applied these assays to cell lines and primary patient samples to refine the list of candidate tumor biomarkers. Candidates of interest detected by MS on MM patient samples were further validated using flow cytometry.

In total, the inventors identified 696 MM cell surface N-glycoproteins by CSC and developed 73 targeted MS detection assays. MS-based validation using primary specimens detected 30 proteins with significantly higher abundance in patient MM cells than controls. Nine of these proteins were identified as potential immunotherapeutic targets, including five that were validated by flow cytometry, confirming their expression on the cell surface of primary MM patient cells. This MM surface N-glycoproteome will be a valuable resource in the development of biomarkers and therapeutics, and the targeted MS assays will useful in the clinic for the diagnosis, stratification, and treatment of MM patients.

### BACKGROUND:

Recent gene expression analyses performed on MM patient samples have defined transcriptome signatures with the potential to improve predictions of disease progression and patient survival.(8, 9) These studies have also identified novel candidate therapeutic targets. However, DNA and RNA-based approaches do not provide critical information, such as protein abundance levels and sub-cellular localization. Proteomic data are potentially more informative in this regard, but they are not as prevalent as transcriptome data for this indication to date. Present cell-surface proteomic approaches are often exclusively based on immunoassays, such as flow cytometry (FCM). FCM is a powerful tool, but it requires prior knowledge of proteins of interest and it can be limited by the availability and quality of antibodies developed for each particular protein. In contrast, mass spectrometry (MS)-based approaches for surface protein discovery, including methods such as cell surface capture (CSC), enable the semi-quantitative detection of hundreds of proteins in an antibody-independent manner. CSC has recently been applied to develop cell surface protein maps for multiple cancer types.(10-15) When they are used together for antigen discovery and validation, MS and FCM are highly complementary techniques.

To date, efforts to define the cell surface proteome of MM have focused on individual cell lines(16, 17), have studied changes in response to one particular therapy(18), or have used methods that are not optimal for detection of low abundance membrane proteins.(19) As a result, knowledge of clinically informative MM cell surface proteins is lacking. In this study, we first used CSC to define the cell surface N-glycoproteome of four MM cell lines. This discovery-based approach identified 696 MM cell surface N-glycoproteins. Next, targeted quantitation of 73 proteins of interest was carried out using parallel reaction monitoring (PRM) analyses of primary MM patient samples. Cell surface abundance of five proteins (CD5, CD98hc, CD147, CD66, and CD205) was further validated on MM patient samples using FCM. This combination of CSC for discovery, and PRM and FCM for validation of selected candidates provides a detailed view of the MM cell surface, including proteins of biological and therapeutic relevance.

### Materials and Methods:

### Cell Culture

RPMI-8226, RPMI-8226/R5, U-266, MM.1R, and RPMI-1788 cell lines were maintained in RPMI-1640 media (Sigma) supplemented with 10% FBS (20% for RPMI-1788) (Gibco) and 1× Penicillin-Streptomycin-Glutamine (Gibco) at 37° C. and 5% C02. An EBV-transformed B-lymphoblastoid cell line (BLCL) was maintained in RPMI-1640 media supplemented with 10% FBS and 1×PSQ. For use in FCM assays, normal donor mononuclear cells (MNCs) were isolated by density gradient centrifugation (Lymphoprep™ and PBMC Isolation Tubes, StemCell Technologies) from cells derived from discarded leukocyte reduction system cones or discarded apheresis products. T and B cells were isolated according to the manufacturer's instructions using CD3+ or CD19+ selection kits, respectively (Stem Cell Technologies). Normal human bone marrow mononuclear cells (BMNCs) were obtained frozen from Stem Cell Technologies.

### Patient Samples

Informed consent was obtained from all MM patients under MCW IRB approval #PRO00027134. Primary human MM cells were obtained from fresh BM aspirates. BMNCs were isolated by density gradient centrifugation (Lymphoprep™ and PBMC Isolation Tubes, StemCell Technologies). CD138+MM cells were isolated using the EasySep™ Human CD138 Positive Selection Kit (StemCell Technologies). CD138+ and CD138− cell fractions were frozen on the day of collection, either as pellets or suspended in freezing media (50% X-VIVO 20 media (Lonza), 40% autologous human serum, and 10% DMSO). Lysis of the cells was required to remove the beads prior to MS analysis.

### Flow Cytometry

Following collection of patient specimens, the following cell subsets were analyzed by FCM: whole BM, BMNCs, CD138+ cells, and CD138− cells. For each sample, at least 200,000 cells were washed and incubated for 30 minutes at 4° C. with CD138-PeCy7, CD38-BV421, CD45-APC, CD19-PE and CD56-FITC, or matching isotype controls (BioLegend) at a 1:200 dilution. Stained cells were washed once and fixed with 1% PFA. For analysis of previously frozen primary CD138+ and CD138− cells, BMNCs, T cells, and B cells, Human TruStain FcX™ Fc Receptor Blocking Solution (BioLegend) was used according to the manufacturer's directions. Cells were then stained with CD5-BV421, CD147-PE/Cy7, CD166-PE, CD205-APC (BioLegend), and CD98hc-FITC (ThermoFisher), or matching isotype controls (BioLegend). All samples were analyzed on a BD LSRFortessa X-20 flow cytometer (BD Biosciences). Data were analyzed using FlowJo software (FlowJo LLC).

### CSC for Discovery of Cell Surface N-Glycoproteins

The CSC technology(20) workflow was performed as previously described(14, 21, 22) with ˜100 million cells per biological replicate (n=3-6). Briefly, cell surface oligosaccharides on live cells were oxidized under mild conditions and labeled with biocytin hydrazide. Following lysis under hypotonic conditions, lysates were depleted of nuclei by differential centrifugation. A membrane-enriched fraction was prepared by ultracentrifugation at 210,000×g for 18 hours. The resulting membrane pellet was digested with trypsin and biotinylated glycopeptides were captured by immobilized streptavidin resin and stringently washed to remove non-specifically bound peptides. Upon digestion with PNGaseF, the peptides were released from the glycan moiety and then subsequently desalted and dried under vacuum. Samples were analyzed using a Q Exactive MS (Thermo; Waltham, Mass.). Data were analyzed using ProteomeDiscoverer 2.2 (Thermo). The exported peptide lists were manually reviewed and proteins that lacked at least one peptide with a deamidated asparagine within the N-linked glycosylation consensus sequence (N-X-S/T/C, where X is any amino acid except proline) were discarded.

### Cell Lysis, Protein Digestion, and Peptide Cleanup

For whole-cell lysate analysis of lymphocyte cell lines and patient samples, pellets of cells were lysed in 500 μL of 2× Invitrosol (40% v/v; Thermo Fisher Scientific) and 20% acetonitrile in 50 mM ammonium bicarbonate. Samples were sonicated (VialTweeter; Hielscher Ultrasonics, Teltow, Germany) by three ten-second pulses, set on ice for one minute, and then re-sonicated. Beads were removed magnetically. Samples were brought to 5 mM TCEP and reduced for 30 min at 37° C. on a Thermomixer at 1200 RPM. Samples were then brought to 10 mM IAA and alkylated for 30 min at 37° C. on a Thermomixer at 1200 RPM in the dark. 20 μg of trypsin was added to each sample; digestion occurred overnight at 37° C. on a Thermomixer at 1200 RPM. Peptides were cleaned by SP2 following a standard protocol.(23)

### Targeted Quantitation of Proteins of Interest Among Cell Lines and Primary Human Cells

All targeted analyses were performed using an Orbitrap Fusion Lumos Tribrid MS (Thermo; for a full description see Supplemental Methods, below). Data were imported into Skyline(24) and chromatographic peaks were extracted from MS2 spectral data for each detected peptide from the target list. Statistical analyses were performed using Student's t-test and plots were generated in GraphPad Prism.

### Data Sharing Statement

Data are available in a public, open access repository. Original mass spectrometry data have been deposited to MassIVE (MSV000084858) and Panorama (panoramaweb.org/MedinMM.url).

### Supplemental Methods

### CSC-Technology

Approximately 100 million cells from each biological replicate (n=3-6) were taken through the CSC-Technology workflow as previously described in detail.(1-3) Cells were washed with PBS and oxidized by treatment with 1 mM sodium meta-periodate (Pierce, Rockford, Ill.) in PBS pH 7.6 for 15 min at 4° C. followed by 2.5 mg/ml biocytin hydrazide (Biotium, Hayward, Calif.) in PBS pH 6.5 for 1 hour at 4° C. Cells were then collected and homogenized in 10 mM Tris pH 7.5, 0.5 mM MgCl₂ and the resulting cell lysate was centrifuged at 800×g for 10 min at 4° C. The supernatant was centrifuged at 210,000×g for 16 hours at 4° C. to collect the membranes. The supernatant was removed and the membrane protein pellet was washed with 25 mM Na₂CO₃ to disrupt peripheral protein interactions. To the resulting membrane pellet, 300 μl 100 mM NH₄HCO₃, 5 mM Tris(2-carboxyethyl) phosphine (Sigma, St. Louis, Mo.), and 0.1% (v/v) Rapigest (Waters, Milford, Mass.) were added and the sample was placed on a Thermomixer (750 rpm) to continuously vortex. Proteins were allowed to reduce for 10 min at 25° C. followed by alklylation with 10 mM iodoacetamide for 30 min. The sample was incubated with 20 μg proteomics grade trypsin (Promega, Madison, Wis.) at 37° C. overnight. Samples were acidified with 5 μl phosphoric acid (88%) then centrifuged at 14,000 rpm for 10 min to remove particulates. The resulting peptide mixture was incubated with 450 μl bead slurry of UltraLink Immobilized Streptavidin PLUS (Pierce, Rockford, Ill.) for 1 hour at 25° C. Beads were sequentially washed with 10 mL each of 0.05% Triton X-100 in 100 mM NH₄HCO₃, 5M NaCl, 100 mM NH₄HCO₃, 100 mM Na₂CO₃, and 80% isopropanol to remove non-specific peptides and lipids. Beads were resuspended in 100 mM NH₄HCO₃ and 500 units glycerol-free endoproteinase PNGaseF (New England Biolabs, Ipswich, Mass.) and incubated at 37° C. overnight with end-over-end rotation to release the peptides from the beads. Collected peptides were desalted and concentrated using a C₁₈ MicroSpin™ column (Harvard Apparatus, Holliston, Mass.) according to manufacturer's instructions.

### Preparation of Whole Cell Lysates for PRM Assays

Cell pellets (approx. 10×10⁶ cells per pellet) were resuspended in 240 μL 100 mM ammonium bicarbonate, 120 μL acetonitrile, and 240 μL Invitrosol LC/MS Protein Solubilizer (5× solution, Thermo Scientific) for 600 μL total lysis buffer volume. This total volume was scaled in equal parts based on cell count, to 200 μL for pellets with 0.8×10⁶-3.7×10⁶ cells or to 1200 μL for pellets with 20×10⁶ cells. Downstream additions of TCEP, iodoacetamide, trypsin, and 10% trifluoroacetic acid (TFA) were also scaled accordingly. Cell suspensions were sonicated (VialTweeter, Hielscher, Teltow, Germany) in microcentrifuge tubes using a 10 s sonication pulse followed by a 10 s pause on ice, with this cycle repeated 10 times total. 33 μL of 100 mM TCEP was added to each tube. Tubes were vortexed then incubated with shaking at 37° C. and 1400 rpm for 30 minutes. 66 μL 100 mM iodoacetamide was added to each tube and tubes were incubated with shaking for another 30 minutes in the dark. 20 μg sequencing grade trypsin (Promega) was added to each tube and digestion proceeded at 37° C. and 1400 rpm overnight. 30 μL 10% TFA was added to each tube to quench digestion. In both cell line and patient sample analyses, a 20 μL aliquot was taken from each sample and cleaned using SP2(4) and eluted into 100 μL 2% acetonitrile 98% water. Peptide concentrations were determined using Pierce Quantitative Fluorometric Peptide Assay (Thermo). Cleaned samples were then diluted with 2% acetonitrile, 98% water with 0.1% formic acid to a final working concentration of 25 ng/μL total sample peptide concentration with Pierce Peptide Retention Time Calibration Mixture (PRTC, Thermo) spiked in, to a final concentration of 1 fmol/μL PRTC. Chromatography, MS and data analysis details are outlined in Tables 9 and 10, below.

### PRM Assay Development

Multiple myeloma cell lines were digested and first analyzed by data dependent acquisition to inform peptide target selection. Three replicate injections, each of 500 ng total peptide, were analyzed by the method outlined in Table 9. MS data were analyzed using Proteome Discoverer 2.2 (Thermo) platform as outlined in Table 10. Identified peptides belonging to proteins of interest (selected from CSC-Technology and intracellular controls) were used to generate a peptide precursor ion target list for follow up parallel reaction monitoring (PRM) analyses. Peptides were selected if they met the following criteria: at least 6 amino acids in length, unique to a master protein accession (i.e. not counting isoforms), and produced at least 5 identified fragment ions in the MS² spectrum. Peptides containing missed tryptic cleavage sites or methionine oxidation were excluded. A maximum of 7 peptides were targeted per protein. All PRM data were analyzed using Skyline(5) software with spectral libraries generated from PD2.2 search results of the DDA data. Chromatographic peak areas are defined by sum of MS² fragment ion signal for identified fragments within a manually surveyed chromatographic peak. Five variations of the normalized collision energy (NCE; 25, 27, 28, 29, or 31) were tested for all peptide targets. For NCE optimization, peptides from all six cell lines were pooled in equal parts and analyzed as two technical injections of 1000 ng total peptide per method. Following analysis in Skyline, the final method was designed to use NCE values that produced the greatest chromatographic peak area for total MS² fragment ion signals.

TABLE 9
Chromatography and MS instrument acquisition settings
for analysis of CSC-Technology Samples

| Sample Volume | 20 | μL |
|---|---|---|
| Stationary Phase | C₁₈ | |
| NanoLC System | Dionex UltiMate 3000 | |
| | RSLCnano | |
| LC Solvent A | 100% H₂O, | |
| | 0.1% formic acid | |
| LC Solvent B | 80% ACN, 20% H₂O, | |
| | 0.1% formic acid | |
| Gradient Ramp | 2.0-27.5% B | |
| Duration | 135 | minutes |
| Flow Rate | 300 | nL/min |
| Mass Spectrometer | Thermo Q Exactive | |
| | Orbitrap | |
| Spray Voltage | 2.0 | kV |
| In-Source CID | 0.0 | eV |
| MS¹ Scan Range | 350-1600 | m/z |
| MS¹ Resolution | 70,000 @ 200 | m/z |
| MS¹ AGC Target | 1e6 | |
| MS¹ Maximum IT | 50 | ms |
| MS² Acquisition | Data dependent, Top 15 | |
| | precursor, Centroid | |
| MS² Fragmentation | HCD | |
| MS² Detection | Orbitrap | |
| MS² Fixed First Mass | — | |
| MS² Resolution | 17,500 @ 200 | m/z |
| Isolation Window | 2.0 | m/z |
| MS² AGC target | 5e4 | |
| MS² Maximum IT | 110 | ms |
| Normalized Collision | 27 | |
| Energy | | |
| Minimum Intensity | 4500 | |
| Req. | | |
| Dynamic Exclusion | 30.0 | s |

TABLE 10
Proteome Discoverer 2.2 search parameters

| Platform | ProteomeDiscoverer 2.2 | |
|---|---|---|
| Search Algorithms | SequestHT, MS | |
| | Amanda 2.0 | |
| Validation | Percolator | |
| | Peptide Validator | |
| | Protein FDR Validator | |
| Database | UniProt; Human; | |
| | created Oct. 3, 2017 | |
| Digest | Trypsin (semi) | |
| | 2 Missed Cleavages | |
| | Allowed | |
| Precursor Mass | 10 | ppm |
| Tolerance | | |
| Fragment Mass | 0.02 | Da |
| Tolerance | | |

| | |
|---|---|
| Static Modifications | Carbamidomethyl (C) |
| Dynamic | Oxidation (M), Acetylation |
| Modifications | (N-terminus) |
| | Deamidation (N) for CSC- |
| | Samples only |
| Target FDR (Strict) | 0.01 |
| for PSMs: | |
| Target FDR (Relaxed) | 0.05 |
| for PSMs: | |
| Target FDR (Strict) | 0.01 |
| for Peptides: | |
| Target FDR (Relaxed) | 0.05 |
| for Peptides: | |

### PRM Assay Application

Whole cell lysate digestions of the six cell lines (n=3 biological replicates) and six multiple myeloma patients (CD138+ and CD138− fractions) were prepared as described above. For all samples, 500 ng total peptide injections were analyzed in technical triplicate. Samples were first analyzed using a data-dependent acquisition method applied to tryptic digests of whole cell lysates to enable selection of unmodified peptides (i.e. not glycopeptides) from cell surface proteins as well as several intracellular proteins of interest. Fully tryptic peptides were selected for parallel reaction monitoring (PRM)(6) assay development and chromatography and normalized collision energies were optimized to obtain at least 10 points across the peak and maximum fragment ion intensity. Skyline(5) was used for all analyses. Overall, suitable results were obtained for 209 peptides from 73 proteins, including 48 cell surface proteins and 14 intracellular controls.

These PRM assays were applied to the same 6 cell lines (n=3 biological replicates each) used for discovery by CSC. To guard against system bias, pooled quality control (Pooled QC) samples were generated by combining equivalent peptides from each of the 6 cell lines per biological replicate and analyzed prior to and after each replicate sample block. Pooled QC samples were generated from respective biological replicate batches. Technical replicate blocks were queued in uniquely randomized order per block. Each block was preceded and followed by analysis of the pooled QC sample. This block/randomization was repeated for each biological replicate. For the primary cells, the six patient samples were blocked by technical replicate, with each fraction analyzed in alternating blocks. Pooled QC samples for each fraction (CD138+, CD138−) were prepared separately and analyzed prior to their respective blocks. A final pooled QC run for CD138+ followed immediately by CD138− was inserted at the end of the overall sample queue. For all PRM analyses, Pierce Peptide Retention Time Calibration Mixture (PRTC, Thermo) was spiked in, to a final concentration of 1 fmol/μL and 500 ng total peptide injections were analyzed in triplicate per sample. Chromatography and MS parameters are described in the Table 11, below.

TABLE 11
Chromatography and MS instrument acquisition
settings for PRM analyses

| Sample Volume | 20 | μL |
|---|---|---|
| Stationary Phase | C₁₈ | |
| NanoLC System | Dionex UltiMate 3000 | |
| | RSLCnano | |
| LC Solvent A | 100% H₂O, | |
| | 0.1% formic acid | |
| LC Solvent B | 80% ACN, 20% H₂O, | |
| | 0.1% formic acid | |
| Gradient Ramp | 2.0-27.5% B | |
| Duration | 135 | minutes |
| Flow Rate | 300 | nL/min |
| Mass Spectrometer | Thermo Orbitrap Fusion | |
| | Lumos | |
| Spray Voltage | 2.1 | kV |
| In-Source CID | 0.0 | eV |
| MS¹ Scan Range | 300-1700 | m/z |
| MS¹ Resolution | 120,000@200 | m/z |
| MS¹ AGC Target | 4e5 | |
| MS¹ Maximum IT | 50 | ms |
| MS² Acquisition | Targeted, Profile | |
| MS² Fragmentation | HCD | |
| MS² Detection | Orbitrap | |
| MS² Scan Range | 120-1200 | m/z |
| MS² Resolution | 30,000 @ 200 | m/z |
| Isolation Window | 1.6 | m/z |
| MS² AGC Target | 1e5 | |
| MS² Maximum IT | 120 | ms |

| | |
|---|---|
| Normalized Collision | 30 |
| Energy (Default) | |

Data were imported into Skyline and chromatographic peaks were extracted from MS² spectra for each detected peptide from the target list. The mean total fragment ion peak areas of the six patients' CD138+ and CD138− samples were compared using a parametric ratio paired t-test using GraphPad Prism. Statistical significance is assigned by p-value<0.05. On graphs, p-value represented by annotations: n. s. for p>0.05, * for p<0.05, ** for p<0.01, *** for p<0.001, **** for p<0.0001.

### Results:

### The Cell Surface N-Glycoproteome of MM Cell Lines

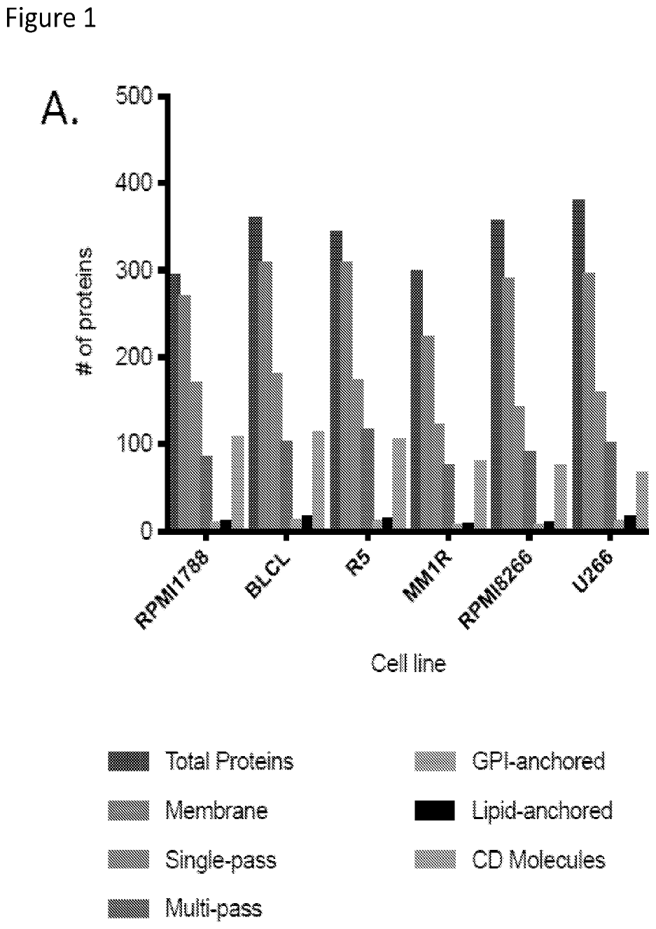

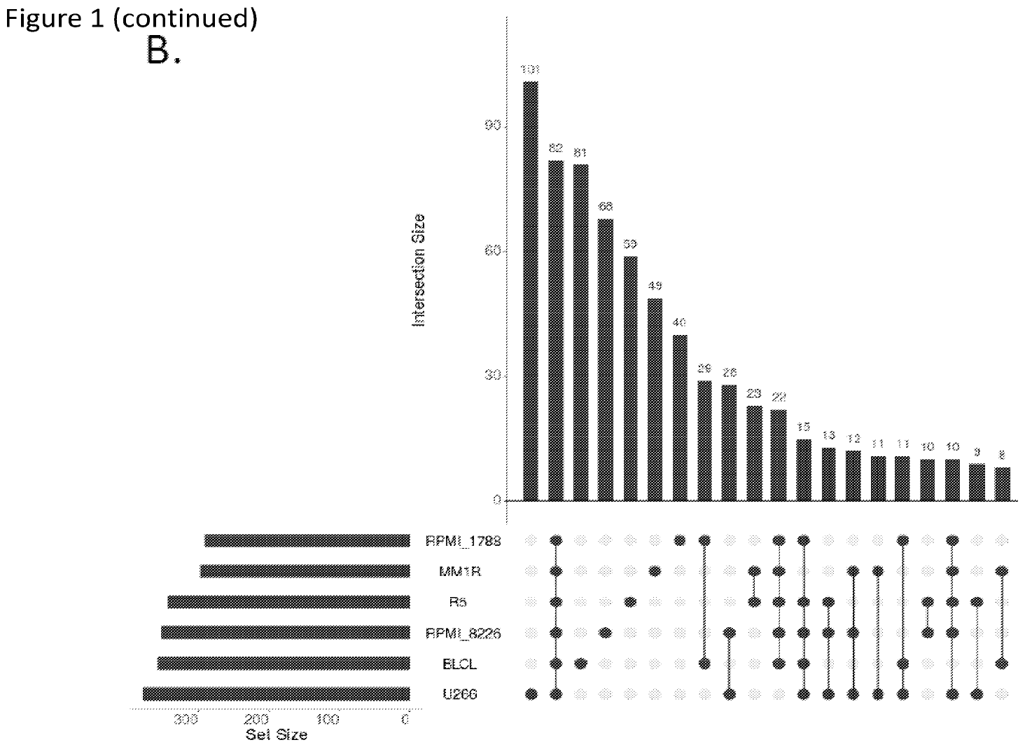

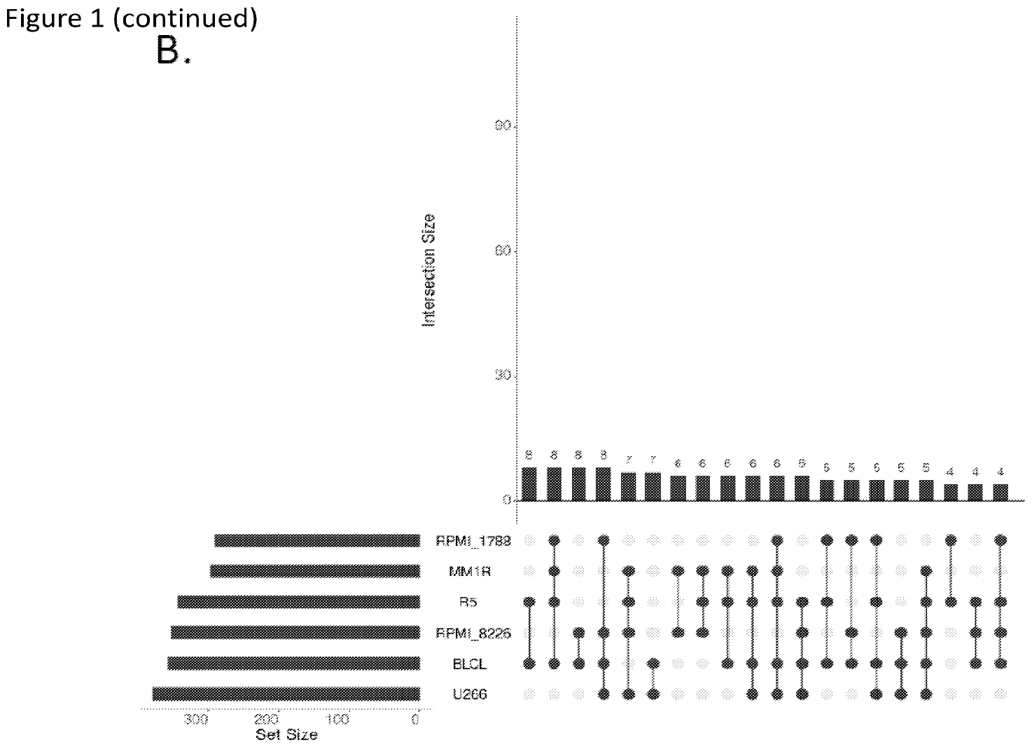

Four cell lines derived from MMN patients (RPMI-8226, RPMI-8226/R5, U-266, MN.fiR) were analyzed. Two B cell lines (RPMJ-1788, BLCL) were included for comparison. By applying CSC technology, 846 distinct cell surface N-glycoproteins were identified, including 171 cluster of differentiation (CD) antigens. The list of 846 N-glycoproteins includes single- and multi-pass membrane proteins, GPI-anchored proteins, and lipid-anchored proteins (FIG. 1A). Overall, 81% of the proteins identified are known to be membrane-associated, demonstrating a high-quality enrichment for surface-localized proteins in the dataset.

Of 696 proteins identified on the 4 MM cell lines, 104 proteins were common to all lines. Many of these 104 proteins were also found on one or both B cell lines, with 7 proteins found exclusively on all 4 MM cell lines (FIG. 1B). This discovery-driven screen identified hematopoietic and B cell markers (e.g., HLA, IgM, CD80), and known MM markers, such as CD38, in addition to proteins not previously described on MM cells.

To further support the utility of our approach for identifying cell surface proteins with relevance to MM, we compared our results to a panel of known MM antigens. Seven proteins known to be informative for immunophenotyping and monitoring of MM (BCMA, CD28, CD33, CD38, CD44, CD45, and CD54) were detected by CSC, as expected. A further 9 proteins (CD19, CD20, CD27, CD52, CD56, CD81, CD 117, CD200, CD307) were not detected, which is consistent with a known lack of expression in MM or expression on cells from only a subset of MM patients.

### Parallel Reaction Monitoring (PRM) Assay Development for MM Markers of Interest

CSC is limited to the detection of extracellular N-glycopeptides and typically requires>50 million cells per experiment. This prohibits application of this approach to routine analysis of primary human cells, especially for patients with low myeloma counts. For these reasons, we applied PRM assays to whole cell lysates from individual MM patient samples, which allowed us to compare the relative abundance of proteins based on the detection of multiple non-modified peptides (which can provide more reliable quantitation than modified peptides) without having to pool patients or expand cells ex vivo. While the analysis of whole cell lysate provides a summary view of the total cell content (not only abundance at the cell surface) for a given protein, PRM assays allow for targeted, quantitative detection of pre-selected proteins with high selectivity and a lower limit of detection than CSC, and require less than 1 million cells. Thus, this approach was applied here to obtain additional evidence of cell type specificity or abundance differences for select proteins prior to subsequent analyses by FCM, which was used to confirm protein abundance at the cell surface.

MM antigens of interest for PRM assay development were identified from the CSC dataset by comparison between the MM and B cell data, along with inclusion of data from the Cell Surface Protein Atlas (CSPA)(15), which is comprised of CSC data from over 80 human and mouse cell lines and primary cells. Publicly available expression databases, such as the Human Protein Atlas(25), were also used as references.

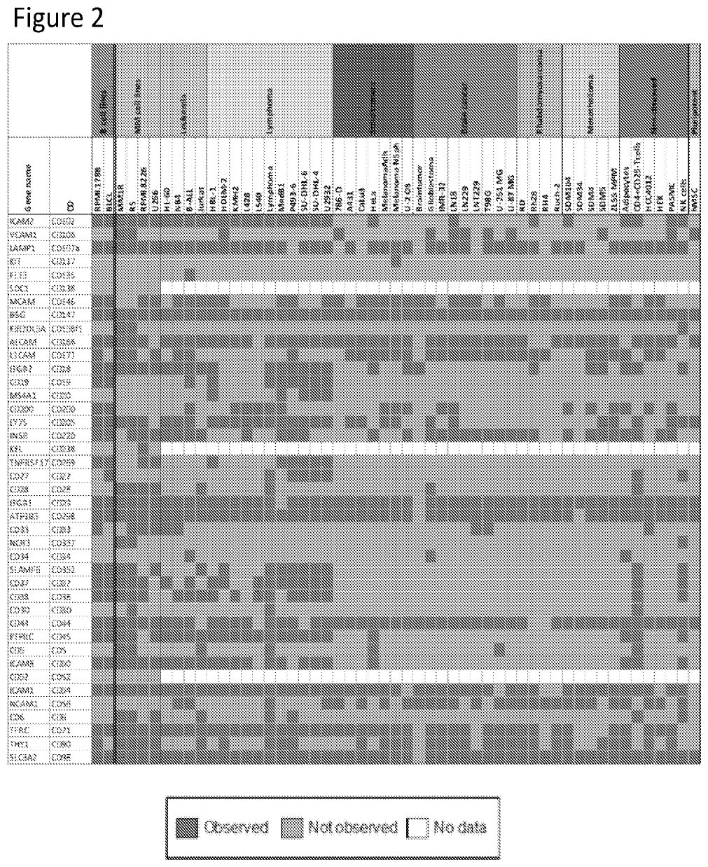

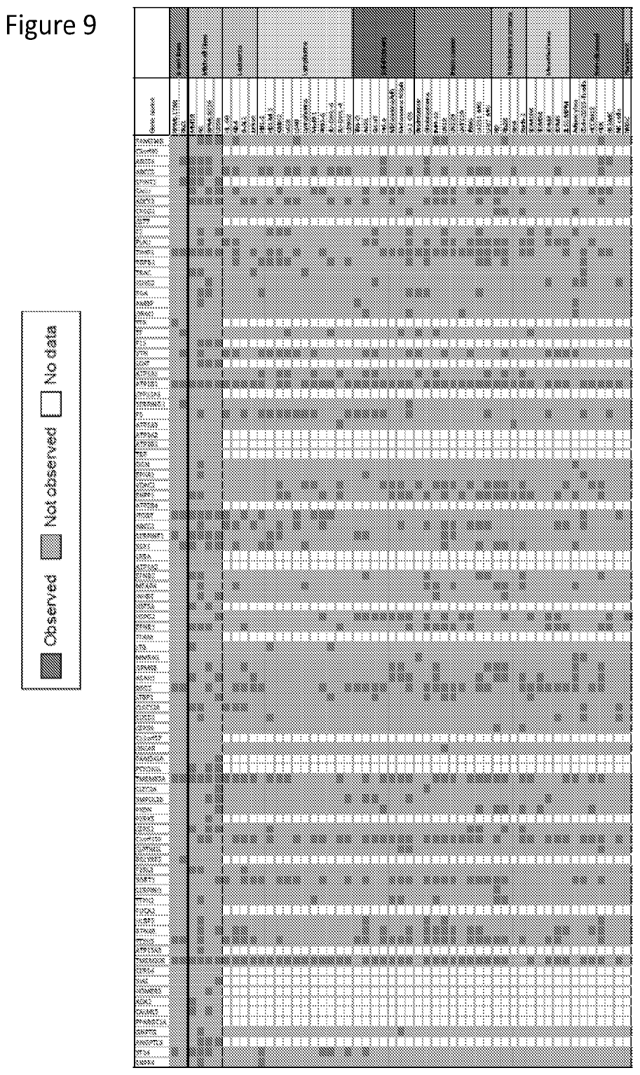

Well-known markers of MM (e.g., CD38, BCMA) were among the proteins selected for PRM assay development. This includes CD138, which was not detected by CSC. As the single predicted N-glycosylation site in CD138 is within a region of the protein that, after trypsin digestion, would yield a peptide that is not detectable with standard analyses, the lack of CD138 detection by CSC is not surprising. Some previously known markers for MM (e.g., SLAMF7, CD305) were identified by CSC at low levels, but were not included for PRM assay development. Proteins expected to be expressed on B cells (e.g., CD19, CD20), therapeutic candidates (e.g., SERCA2, CD28, CD54, CD147), and diagnostic/prognostic candidates (e.g., SLC3A2, CD5, CD90) were also chosen. Other selections included proteins involved in BM homing/bone disease and calcium binding/transport (MM patients often present with osteolytic lesions and bone pain), and proteins involved in cell migration, adhesion, and drug resistance. Several proteins of interest discovered during preliminary data analysis (not shown), but ultimately not identified by CSC as present in the MM cell lines (including CLPTML1, LRBA and others), were also included as candidates for PRM development. Overall, 133 proteins were chosen for further study. The CD and non-CD proteins selected for PRM assay development are listed in FIG. 2 and FIG. 9, respectively, and in each case their previous observation among various cell types in the CSPA are indicated.

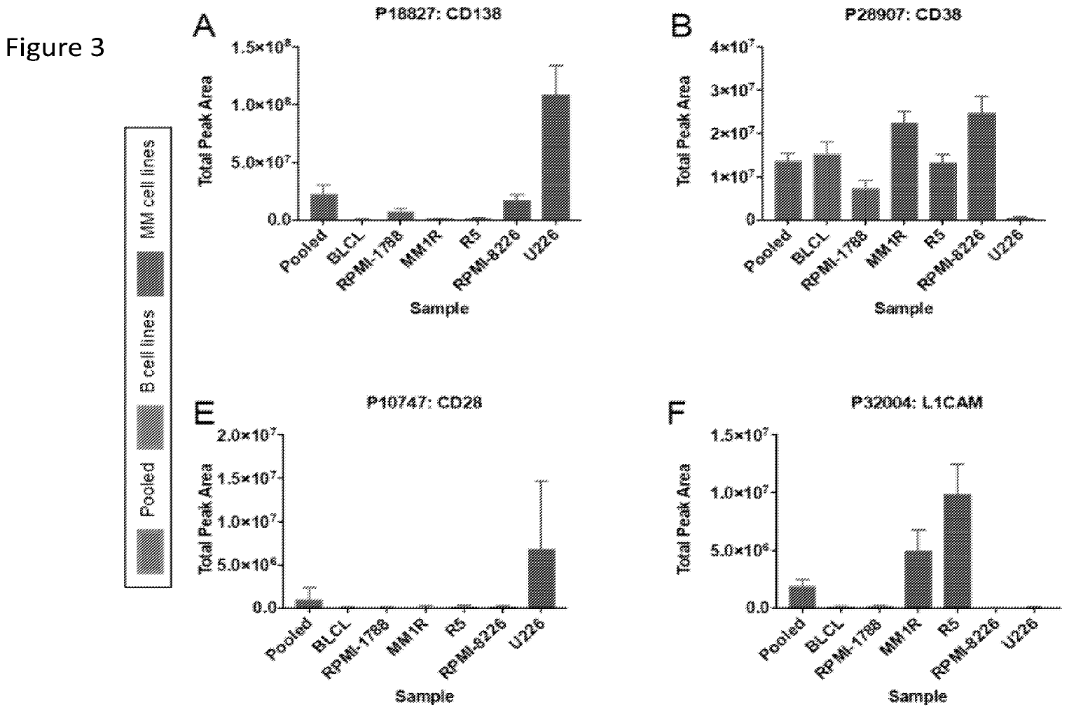

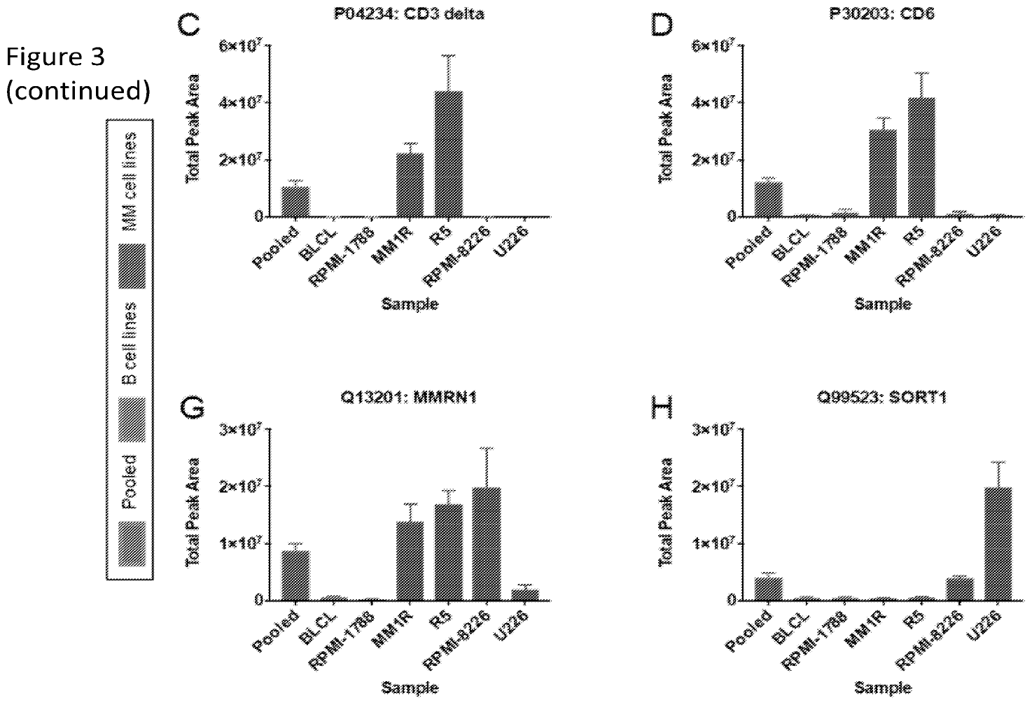

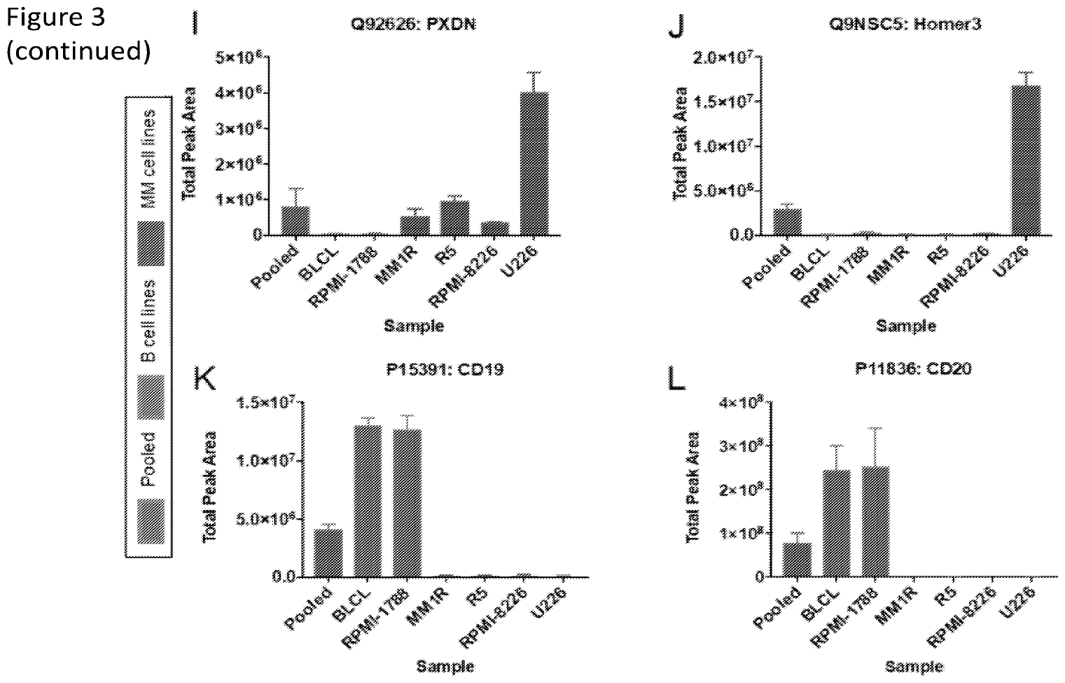

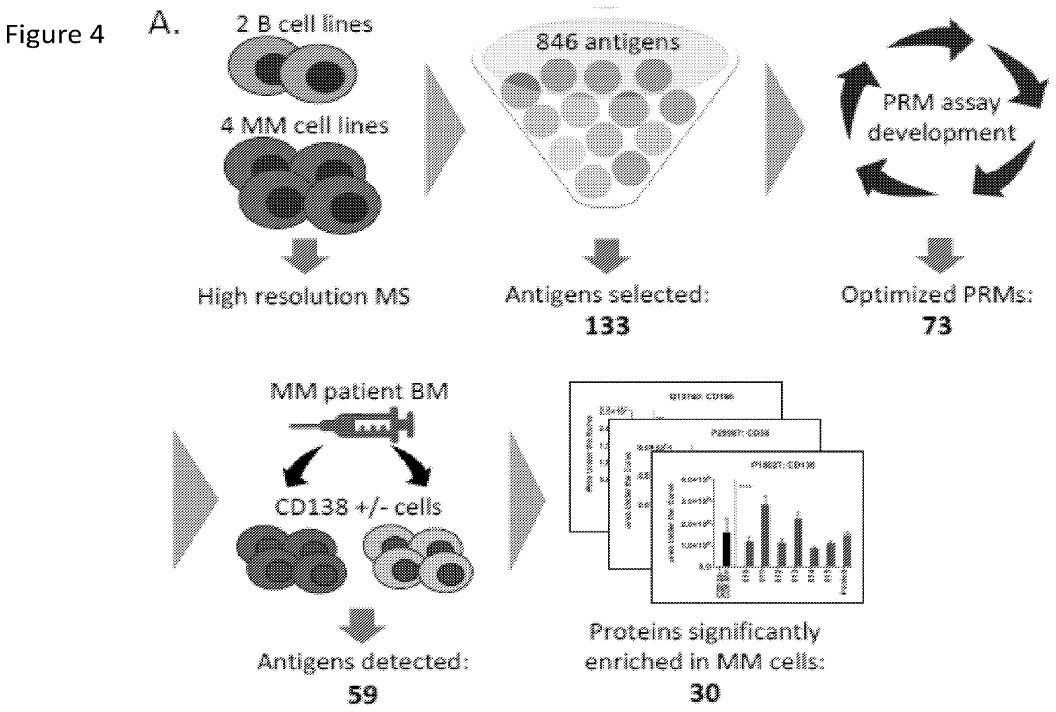

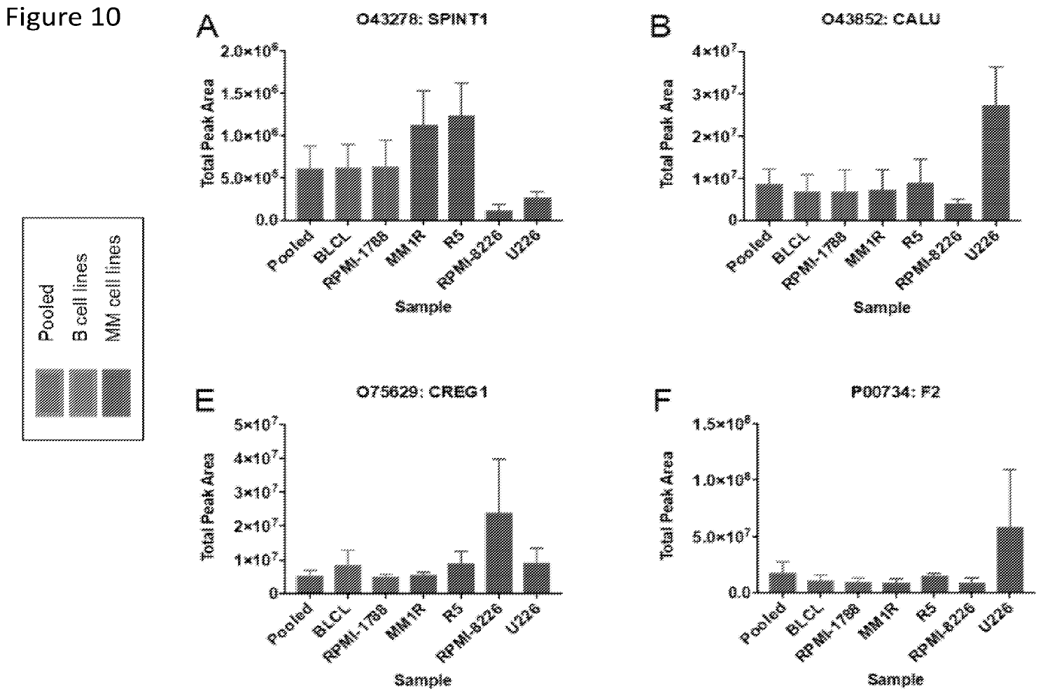

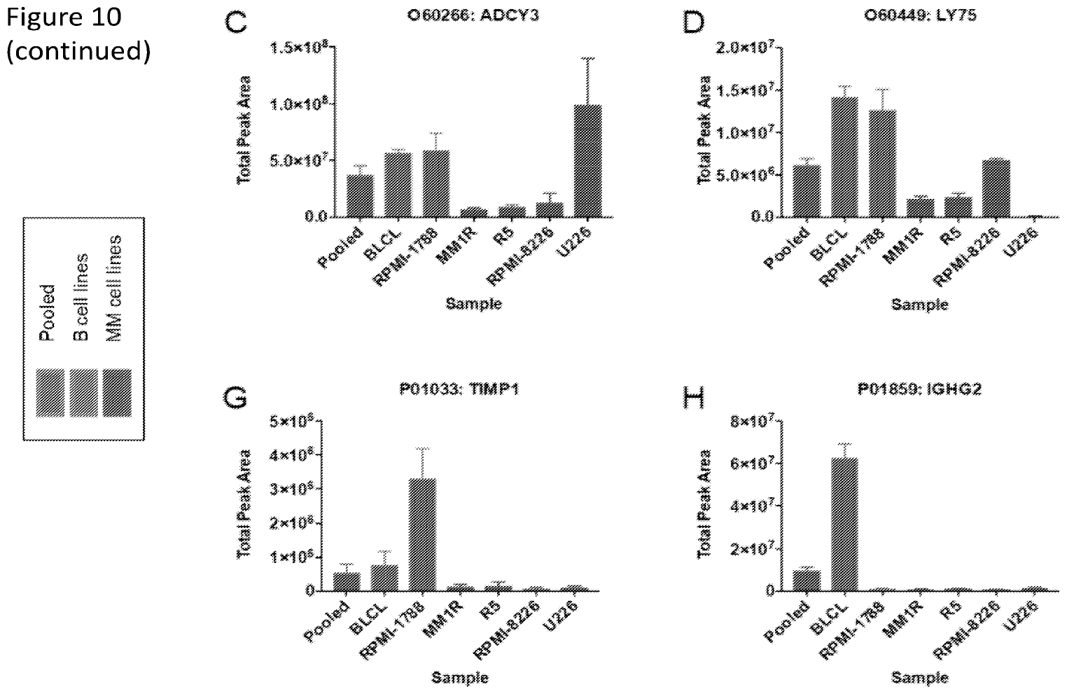

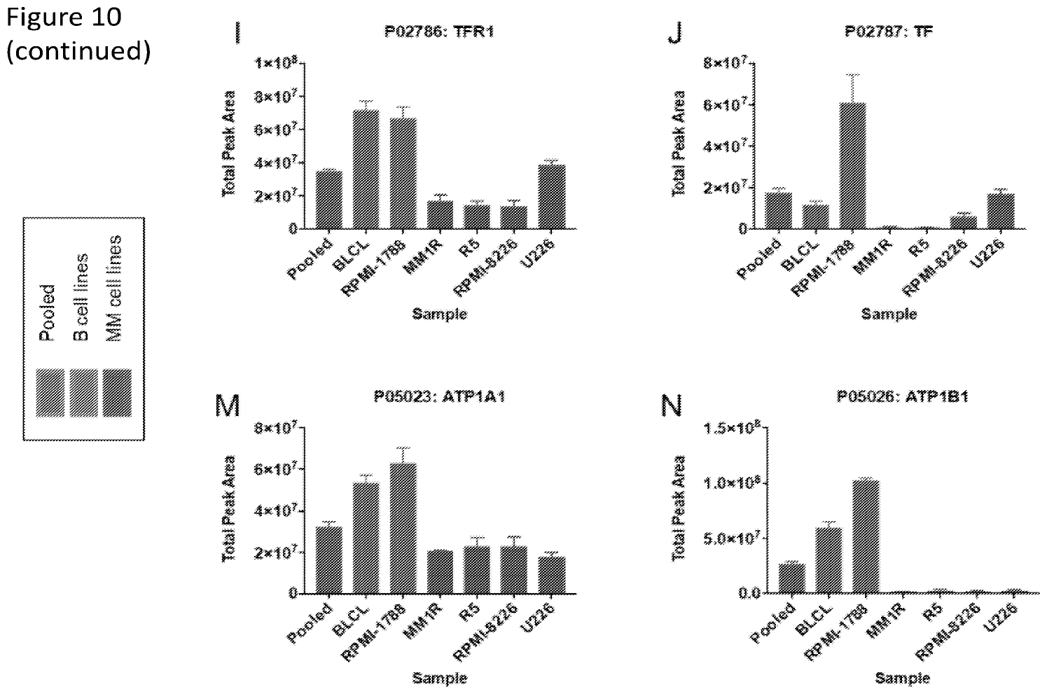

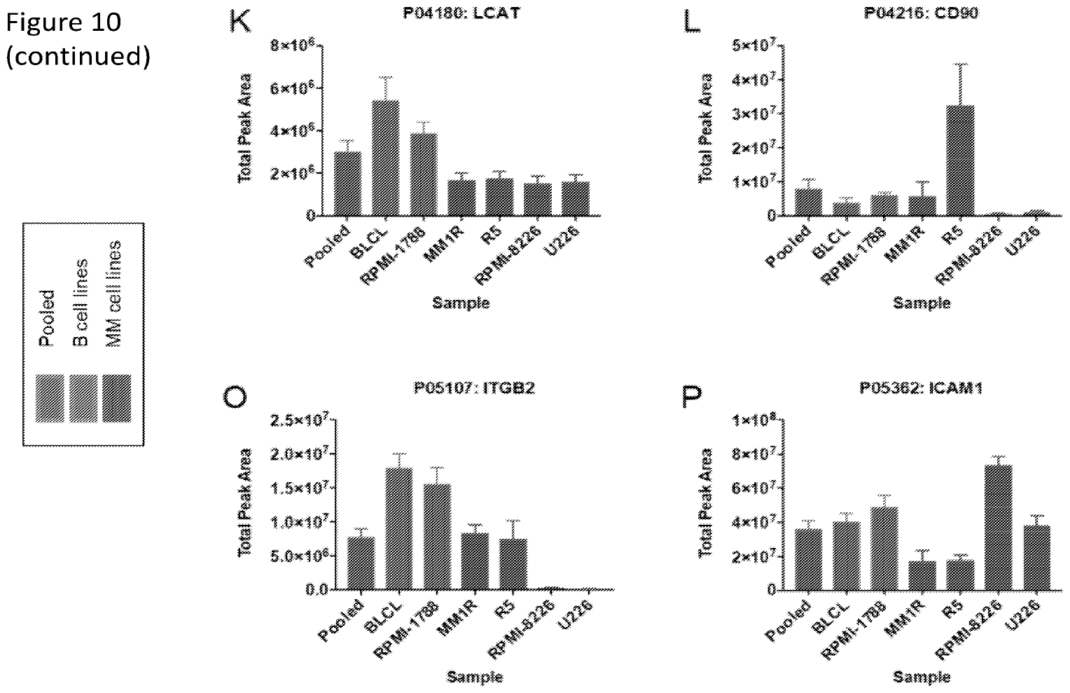

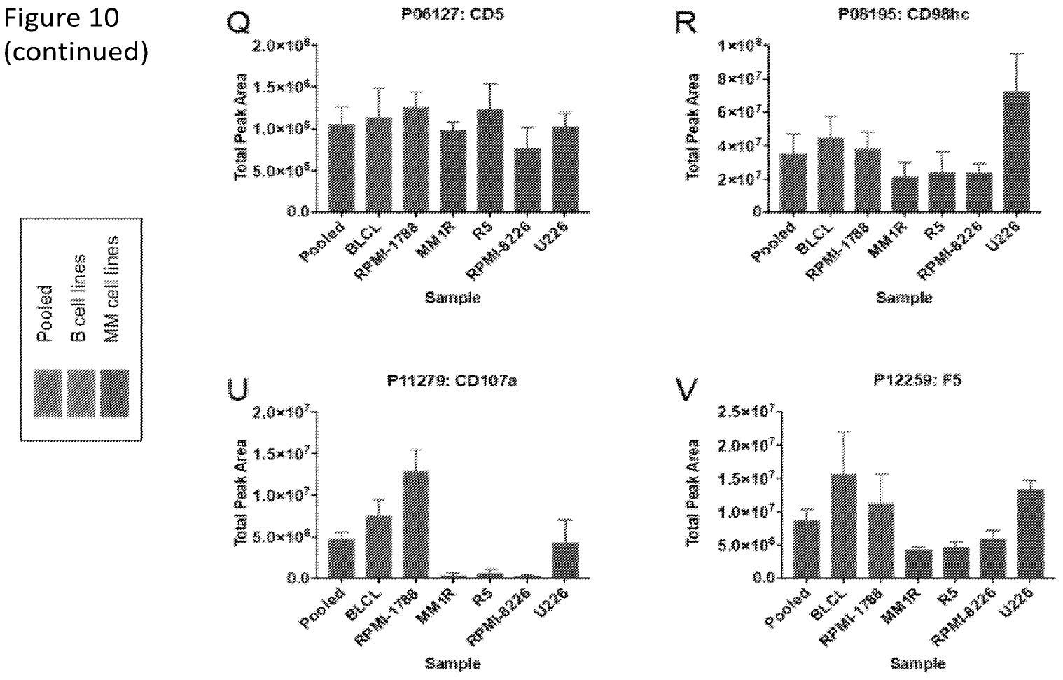

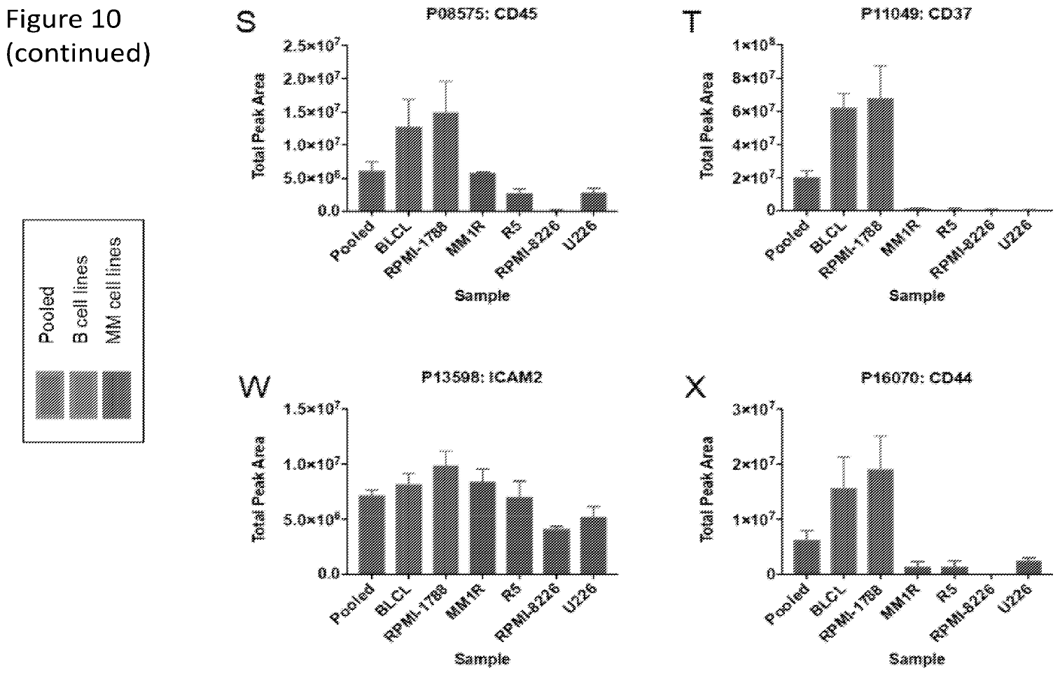

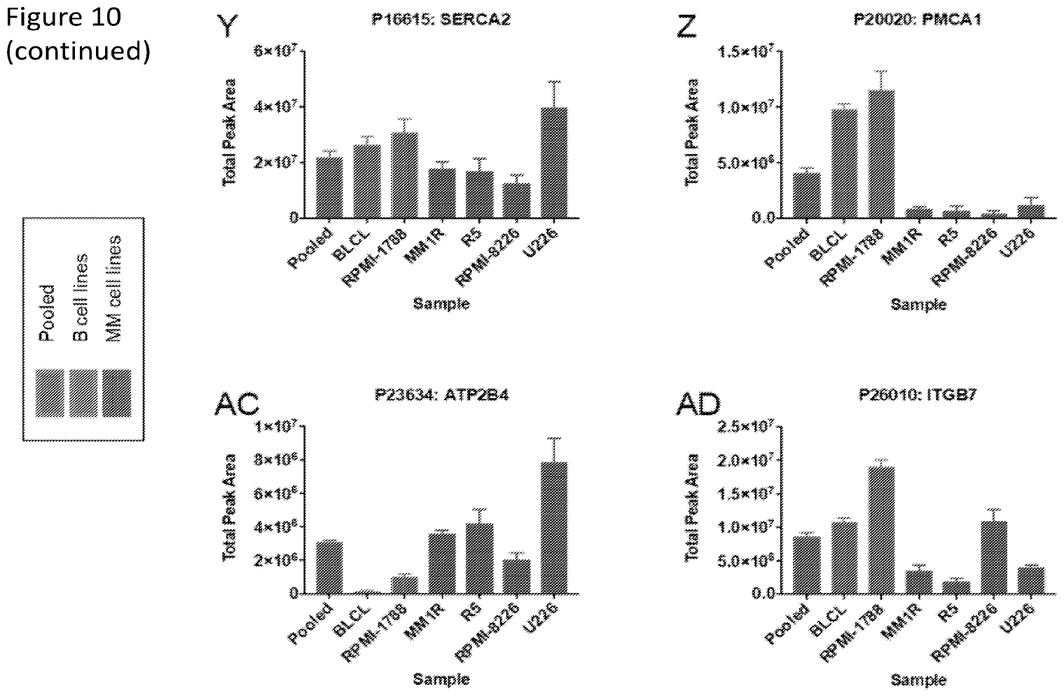

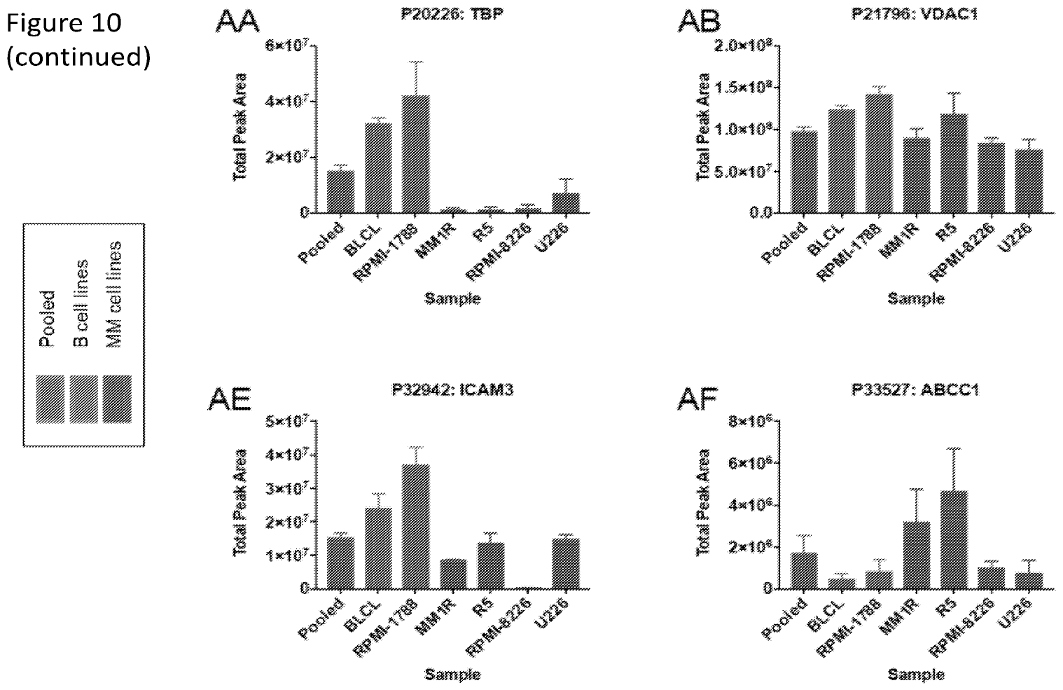

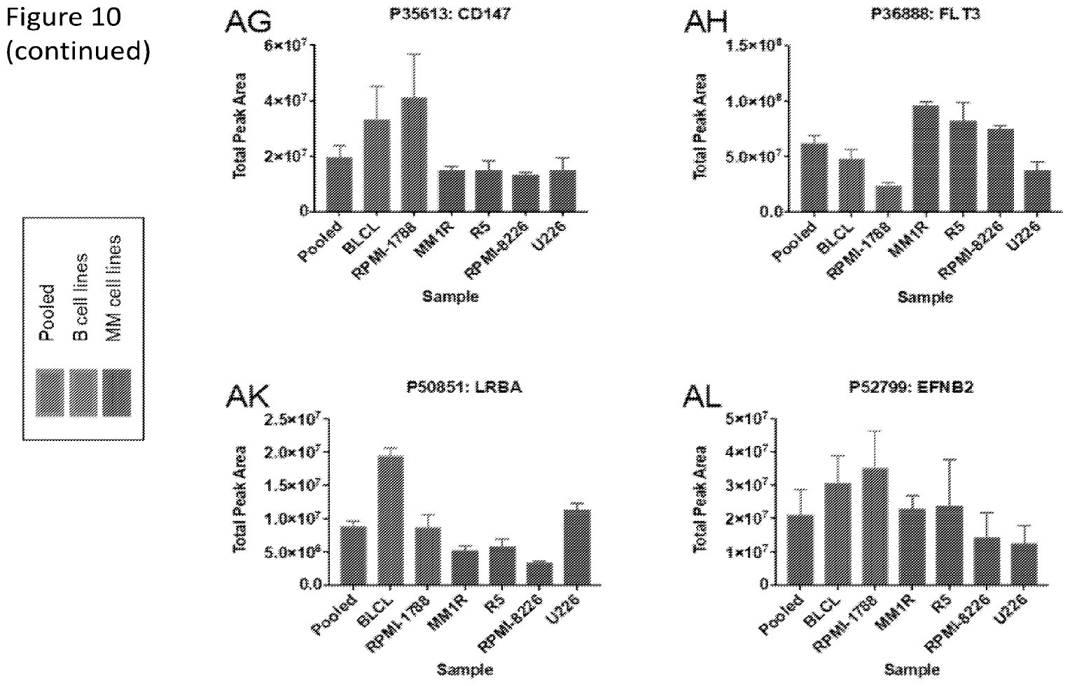

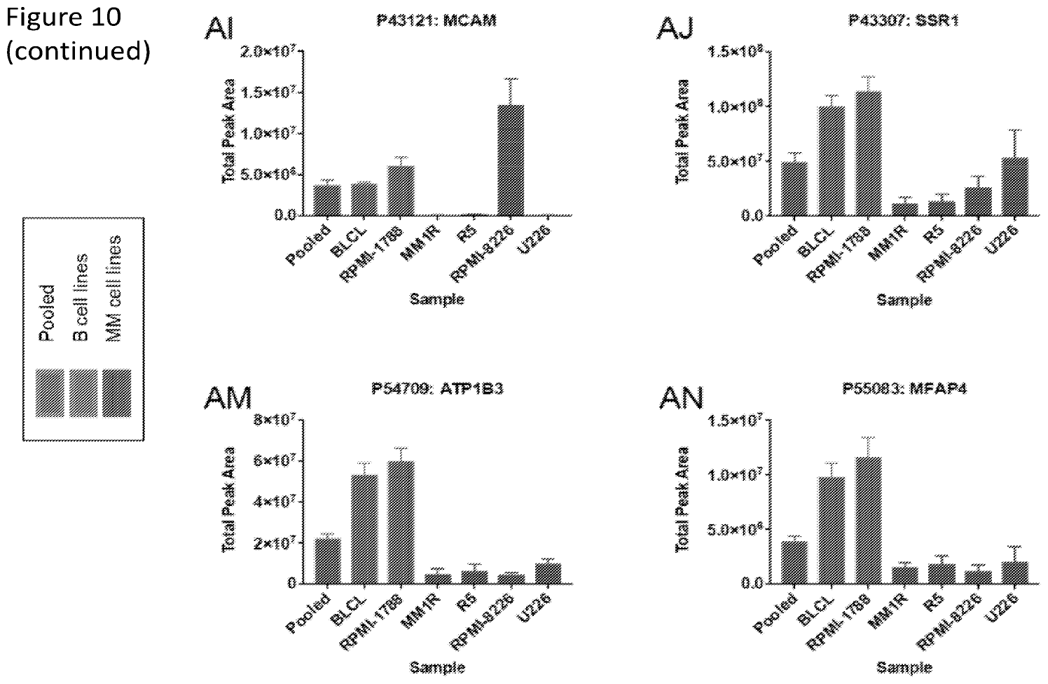

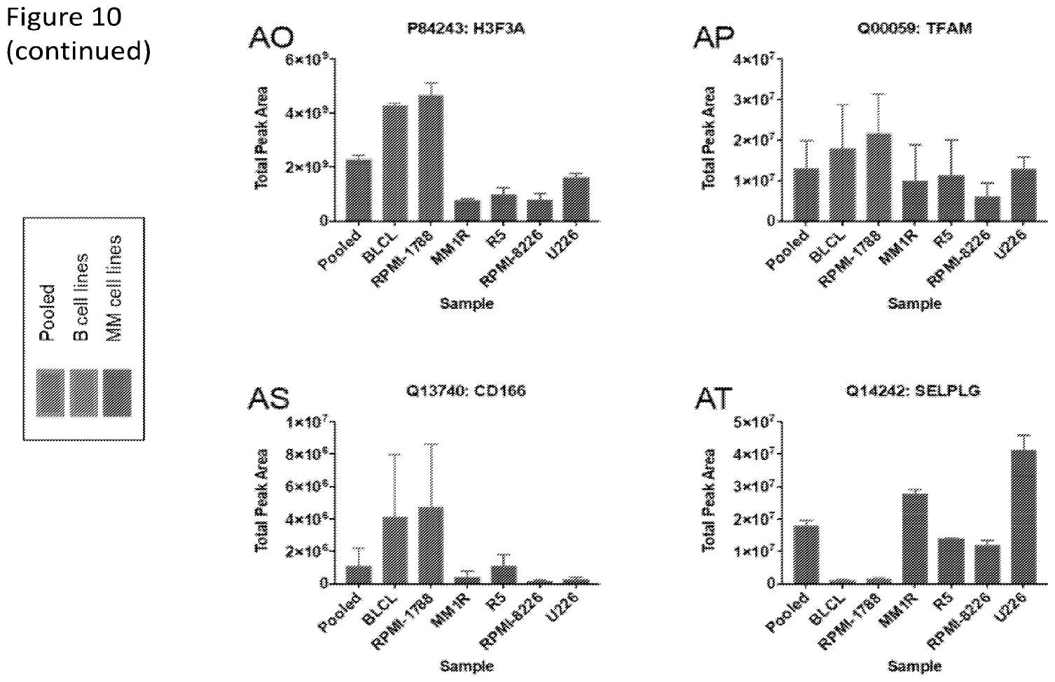

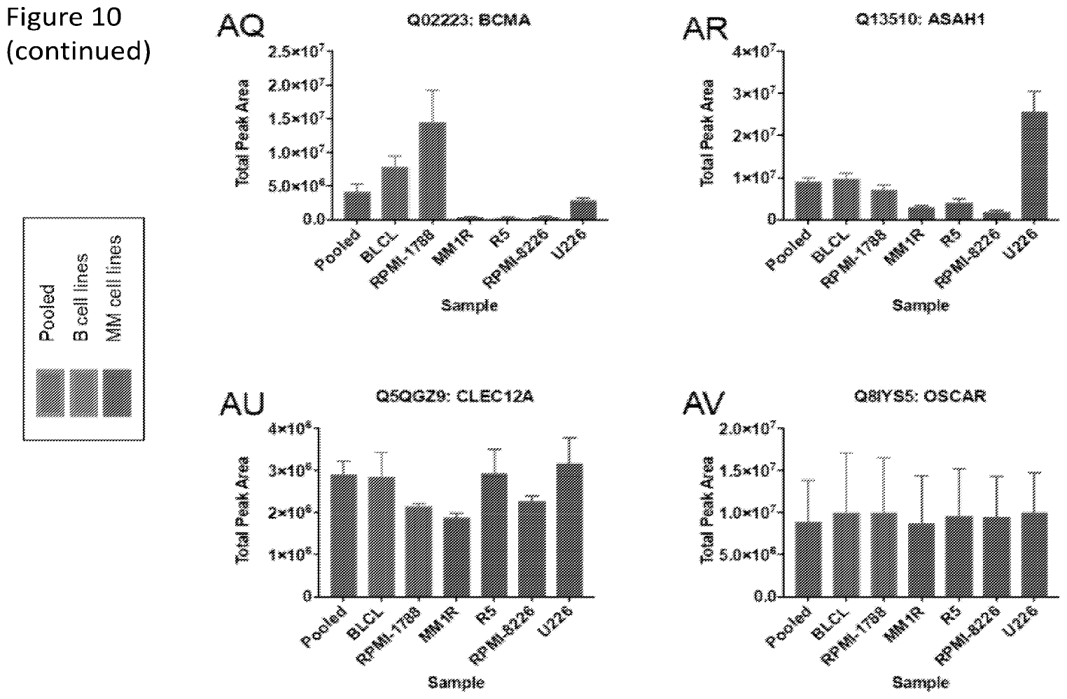

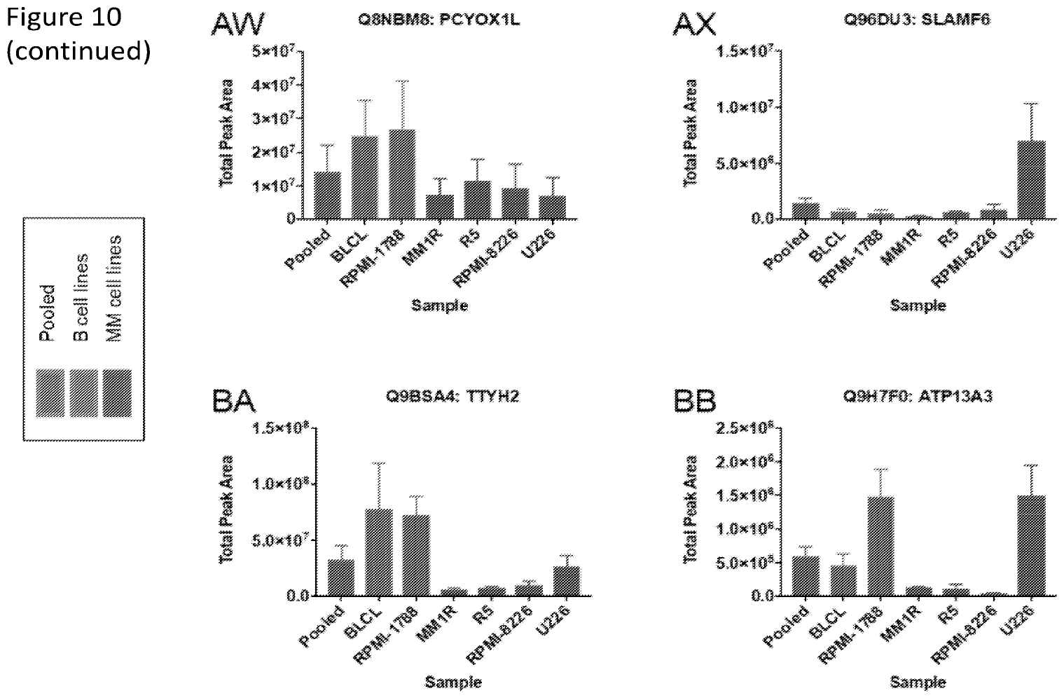

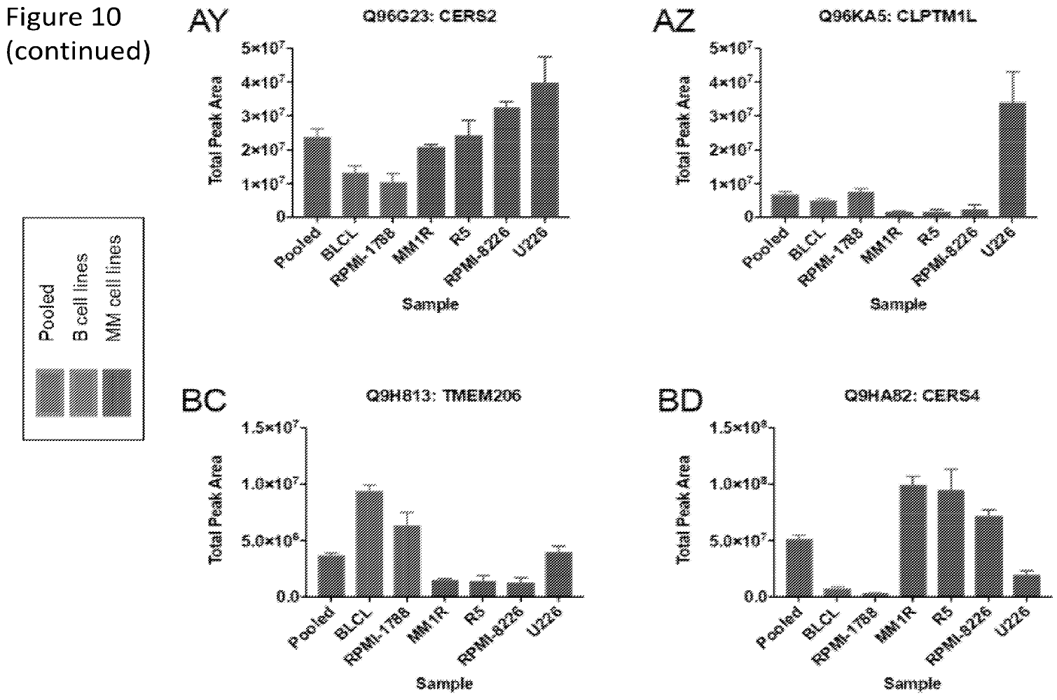

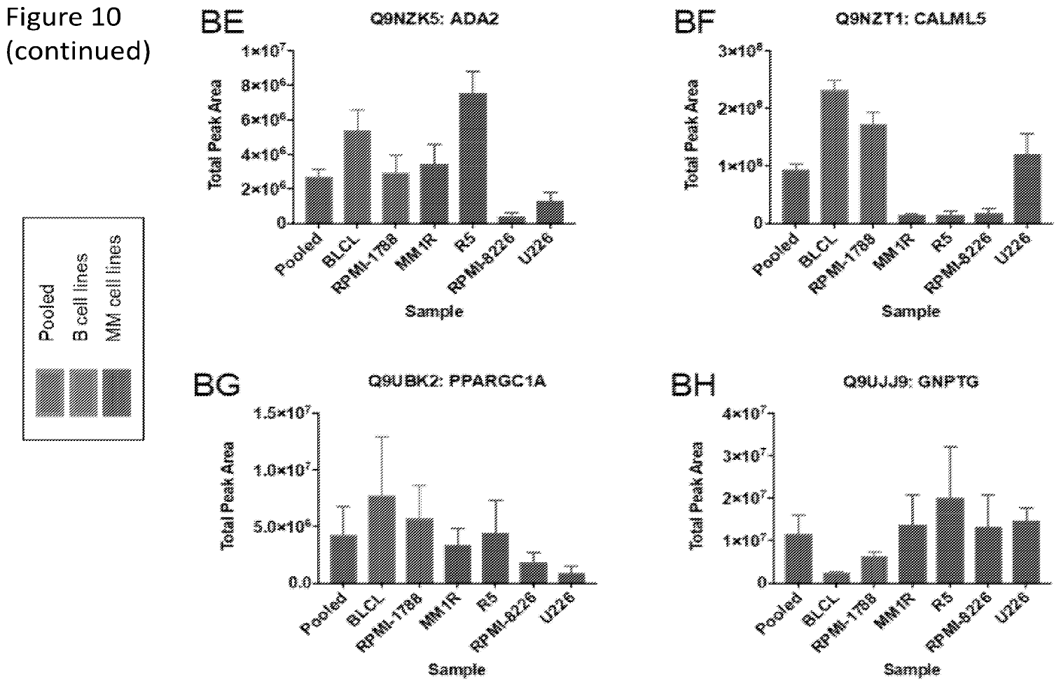

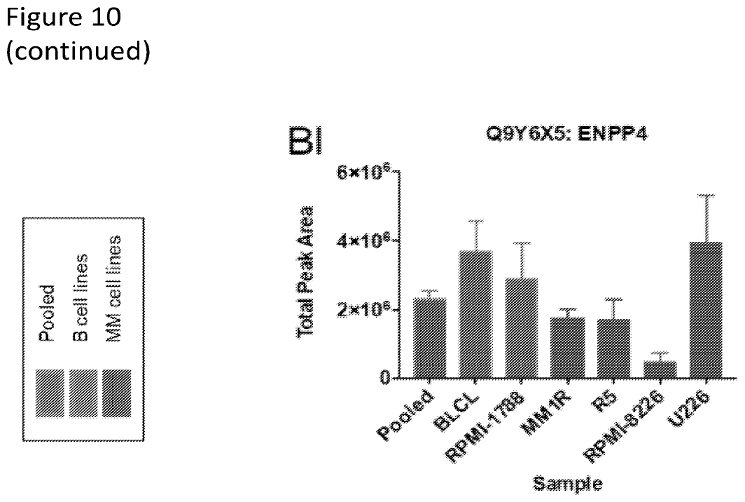

Of the 133 proteins selected, PRM assays were successfully developed for 73 candidates and applied to the MM and B cell lines for validation (FIG. 3; FIG. 10), where all 73 antigens were detected. As expected, proteins such as CD19 and CD20 were found exclusively on the B cell lines. CD138, CD38, CD45, and CD54 were detected at varying levels across both the MM and B cell lines. Detection of BCMA (TNFRSF17) in MM cells was lower than anticipated in comparison to the B cell lines, but expression on the B cell lines was not unexpected since both the RPMI1788 and BLCL cell lines are EBV+, a factor that has been associated with BCMA expression.(26) Proteins that were detected exclusively in the MM cell lines included CD3, CD6, CD28, L1CAM, MMRN1, SORT1, PXDN, and Homer3.

### Assessment of Selected Surfaceome Proteins in Primary MM Samples

Primary BM was obtained from 10 MM patients (a first cohort of 6 patients, and a second cohort of 4 patients) who presented with 2-53% (average 26.7) plasma cells in the BM by clinically diagnostic BM differential cell count. Between 1×10⁶ and 48×10⁶ (average 17.9×10⁶) MM cells per patient were enriched by CD138+ selection to a purity of 23.3-98.9% (average 71.20%), as determined by FCM (Table 1). All patient samples were CD138+ and CD38+, and just one of the 10 was CD19+. Three of the 10 patient specimens were CD56+.

TABLE 1
Patient sample characteristics

| | Cells | % Plasma in | % | |
|---|---|---|---|---|
| | recovered | BM | Purity | Phenotype |
| Cohort 1 | | | | |
| Patient 10 | 48.0 × 10⁶ | 12.6 | 98.9 | No data |
| Patient 11 | 5.0 × 10⁶ | 2.0 | 86.9 | CD56−, CD19− |
| Patient 12 | 41.4 × 10⁶ | 51.2 | 83.8 | CD56−, CD19− |
| Patient 13 | 2.1 × 10⁶ | 52.6 | 89.0 | CD56−, CD19− |
| Patient 14 | 1.0 × 10⁶ | 20.8 | 23.3 | CD56+, CD19− |
| Patient 15 | 2.3 × 10⁶ | 20.8 | 32.8 | CD56+, CD19− |
| Cohort 2 | | | | |
| Patient 16 | 35.0 × 10⁶ | 32.2 | 92.5 | CD56−, CD19− |
| Patient 17 | 2.3 × 10⁶ | 2.2 | 27.8 | CD56−, CD19+ |
| Patient 18 | 15.4 × 10⁶ | 48.4 | 84.5 | CD56−, CD19− |
| Patient 19 | 26.3 × 10⁶ | 24.4 | 92.4 | CD56+, CD19− |

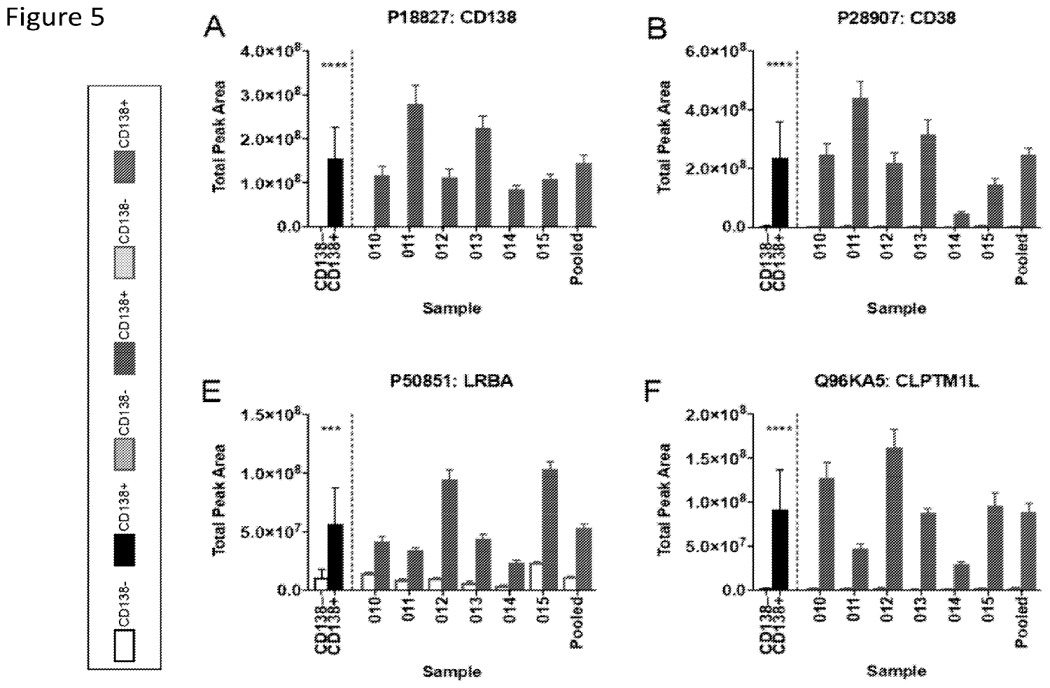

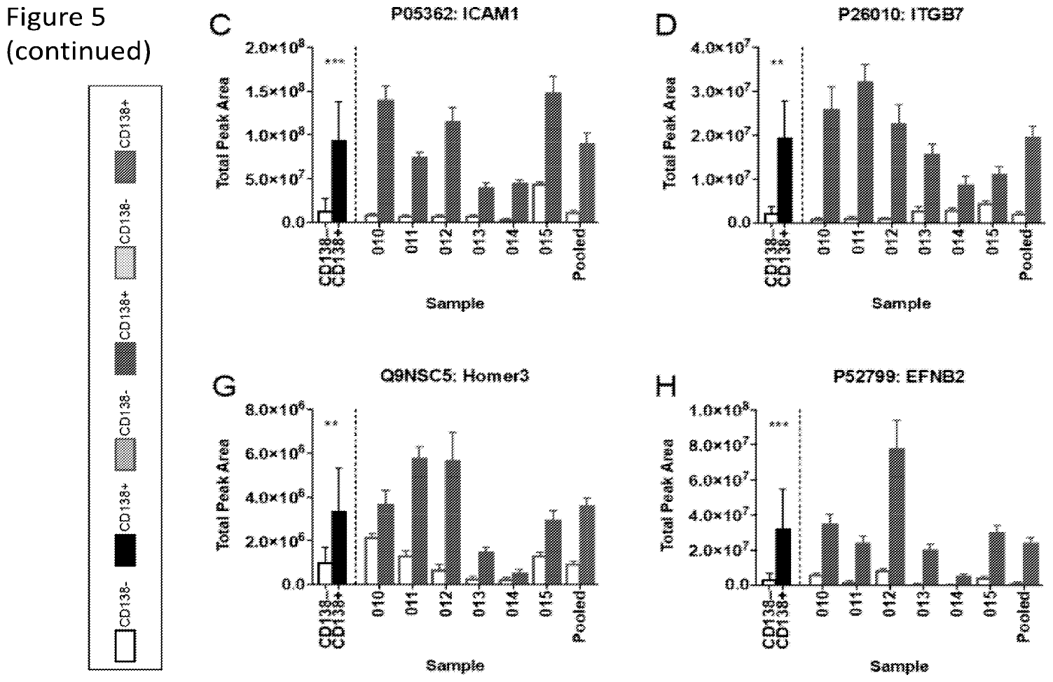

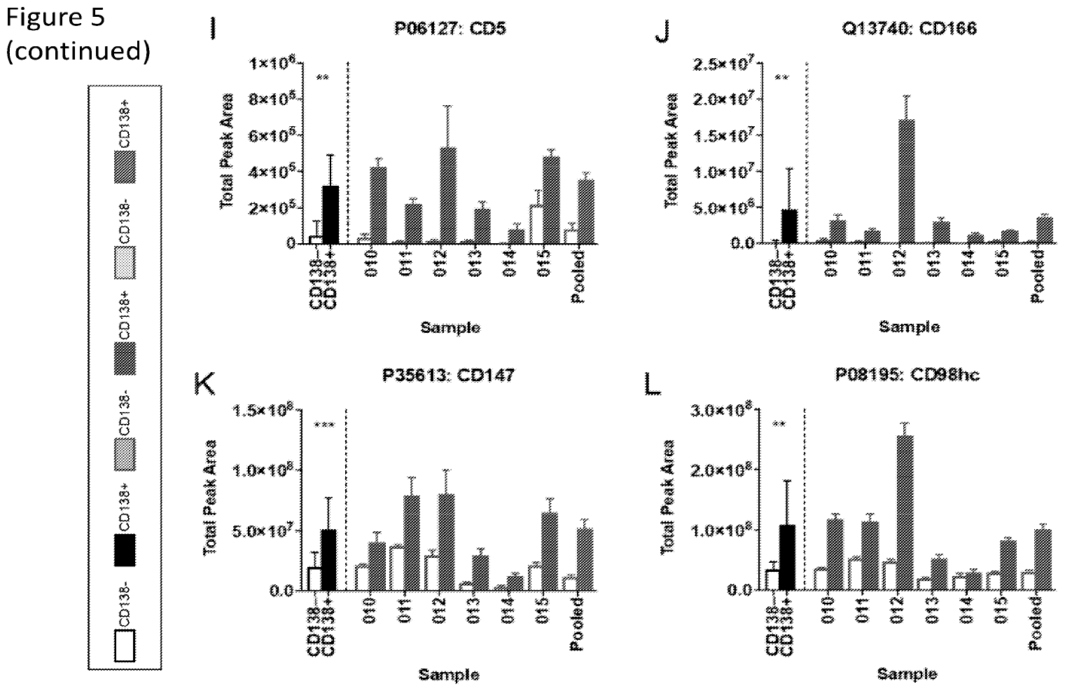

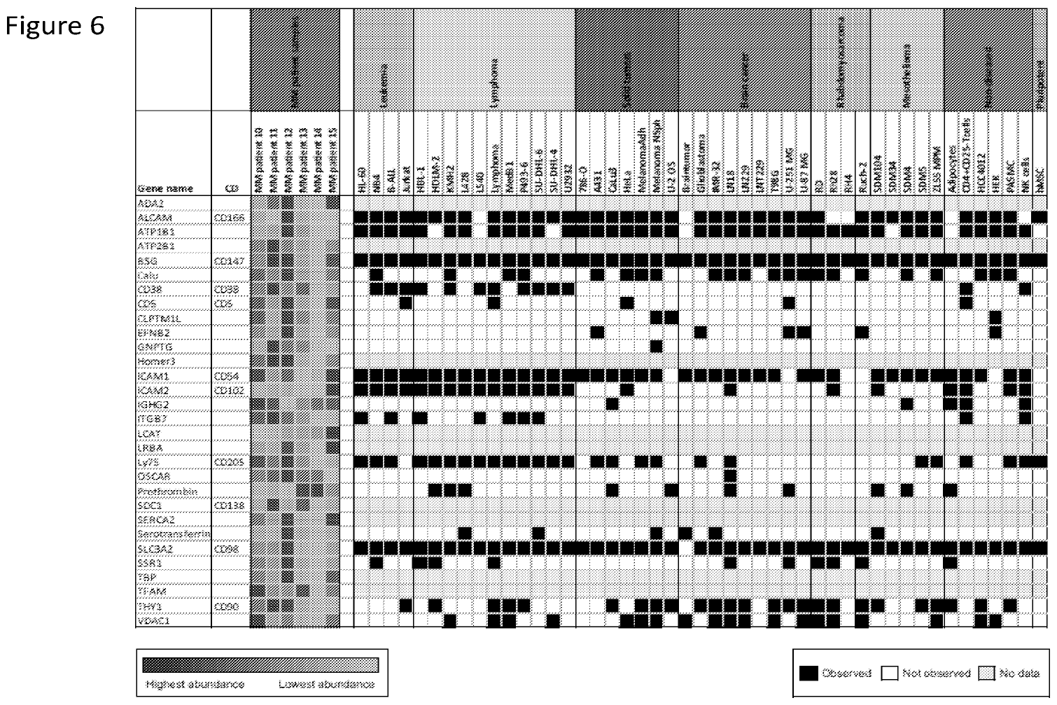

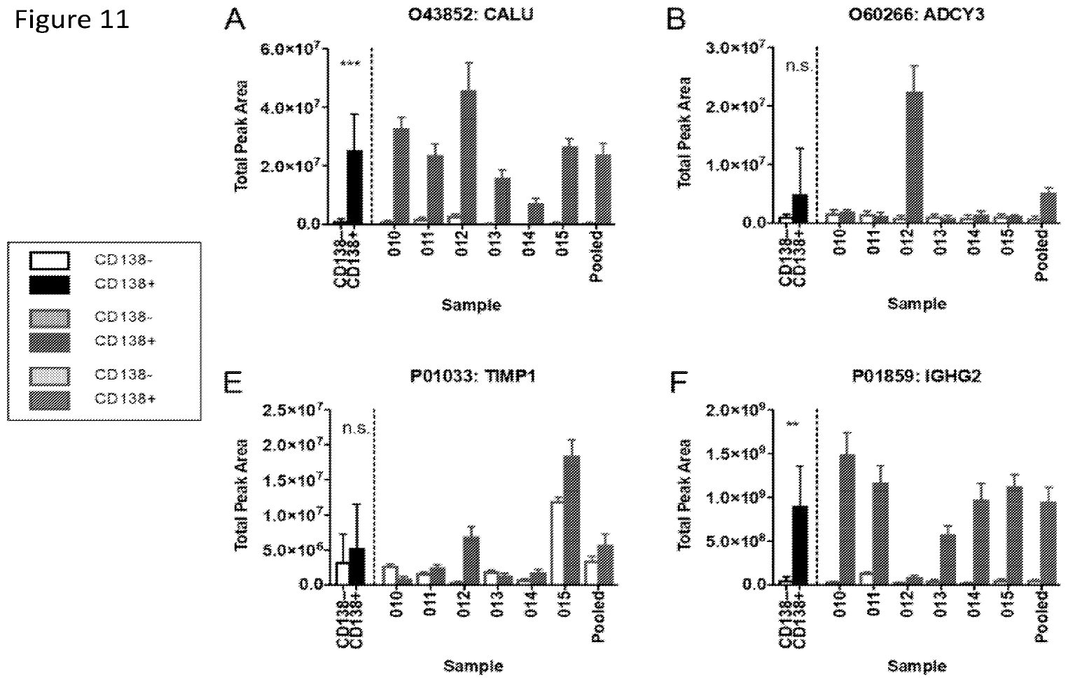

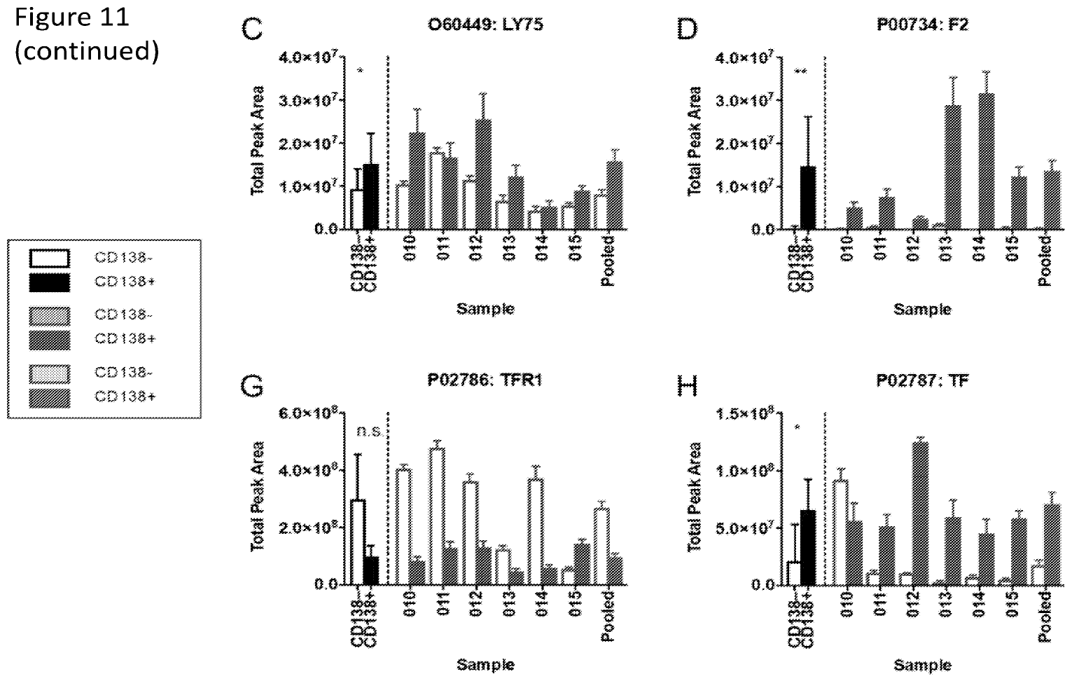

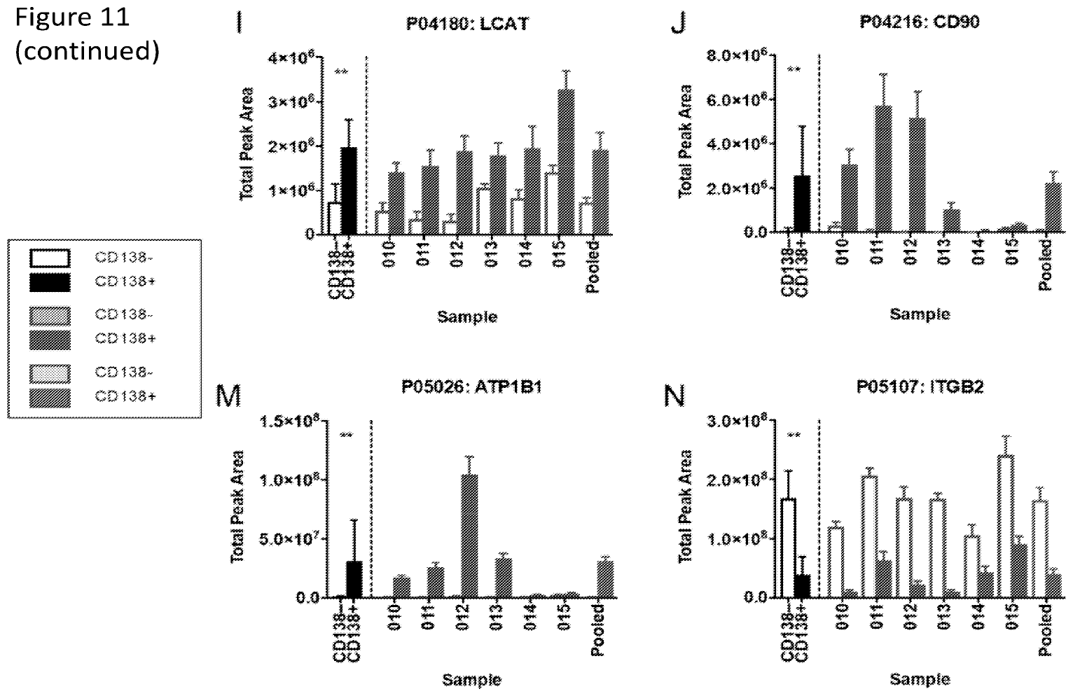

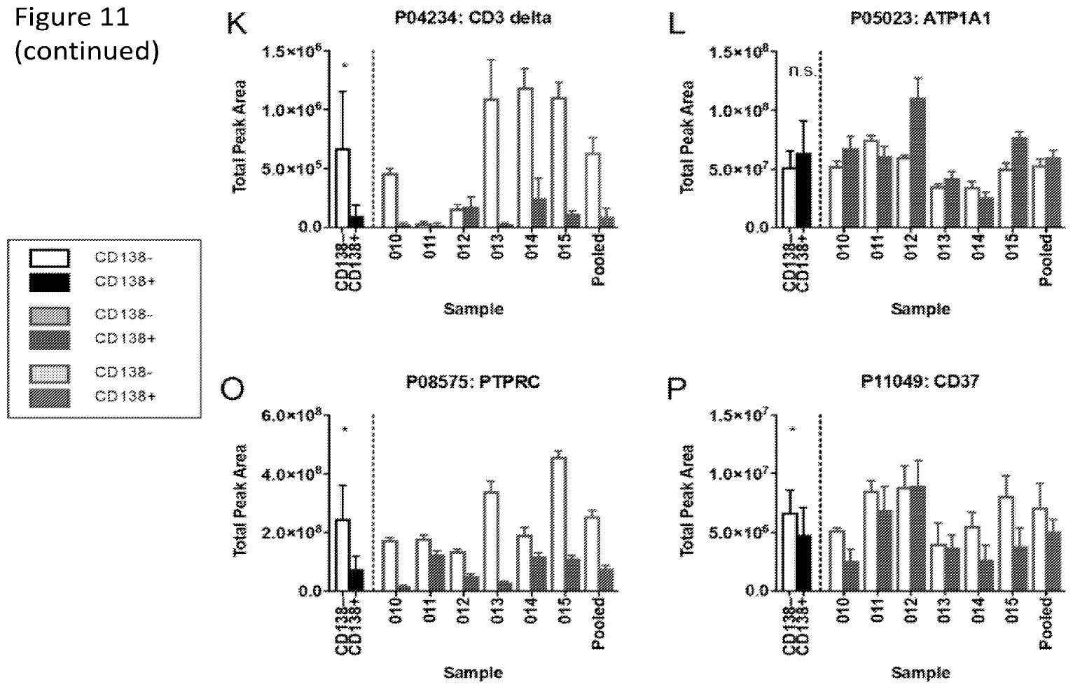

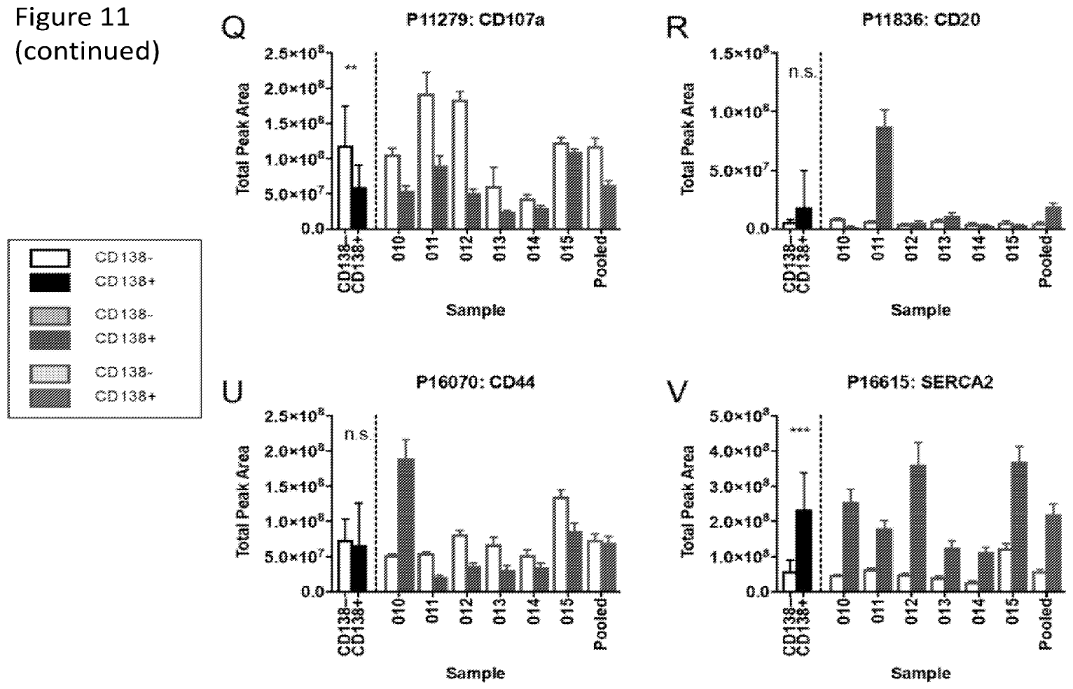

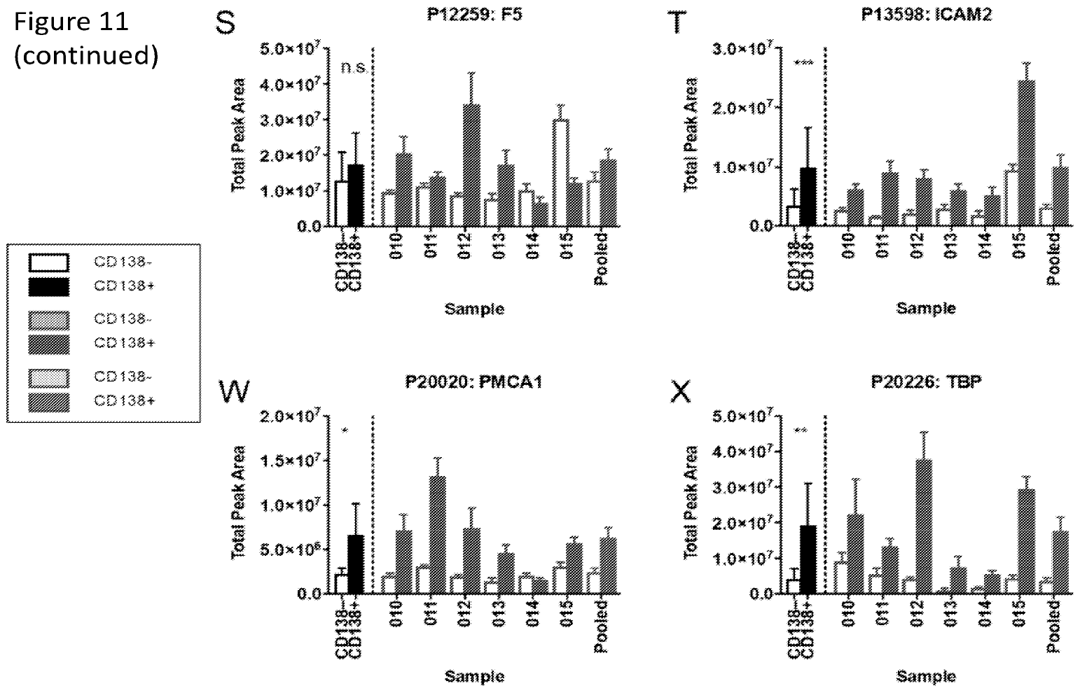

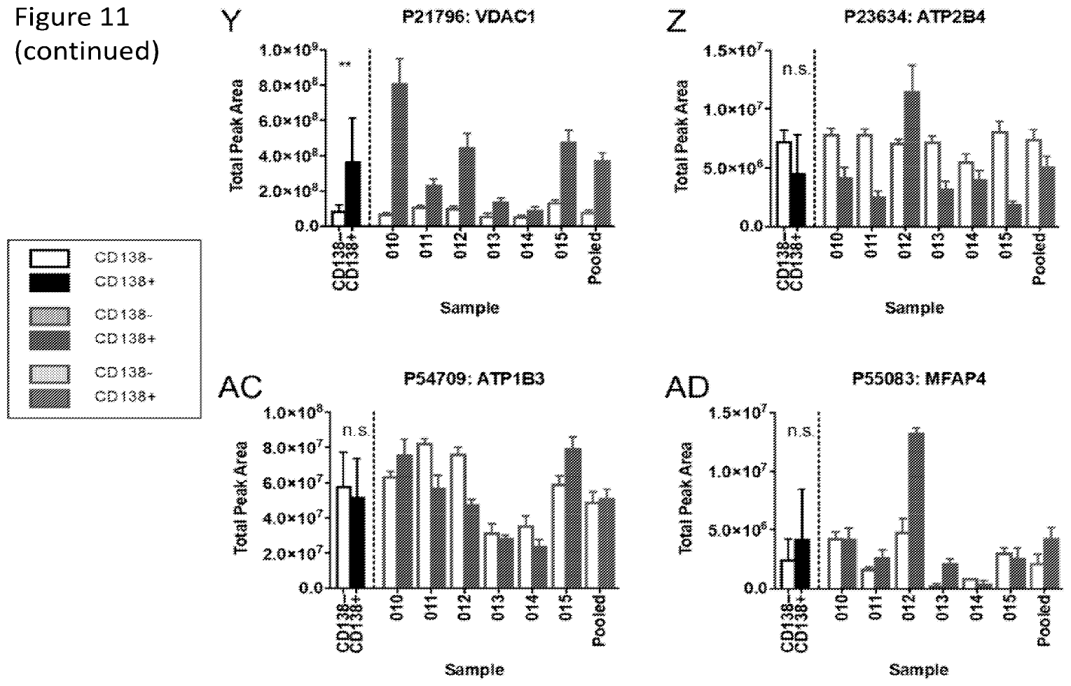

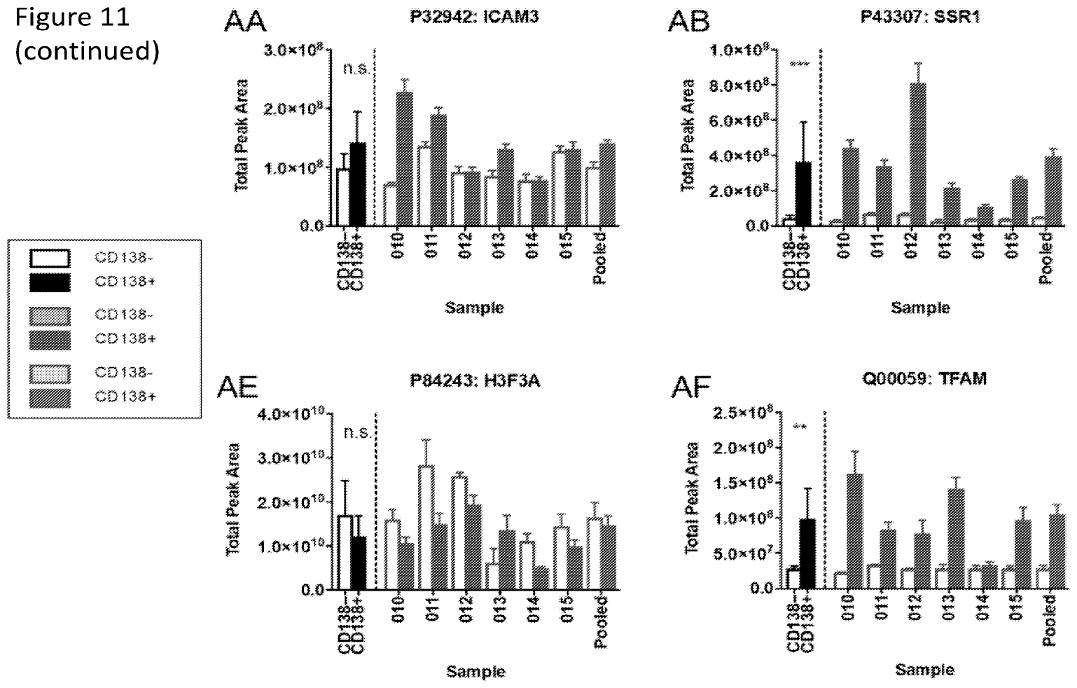

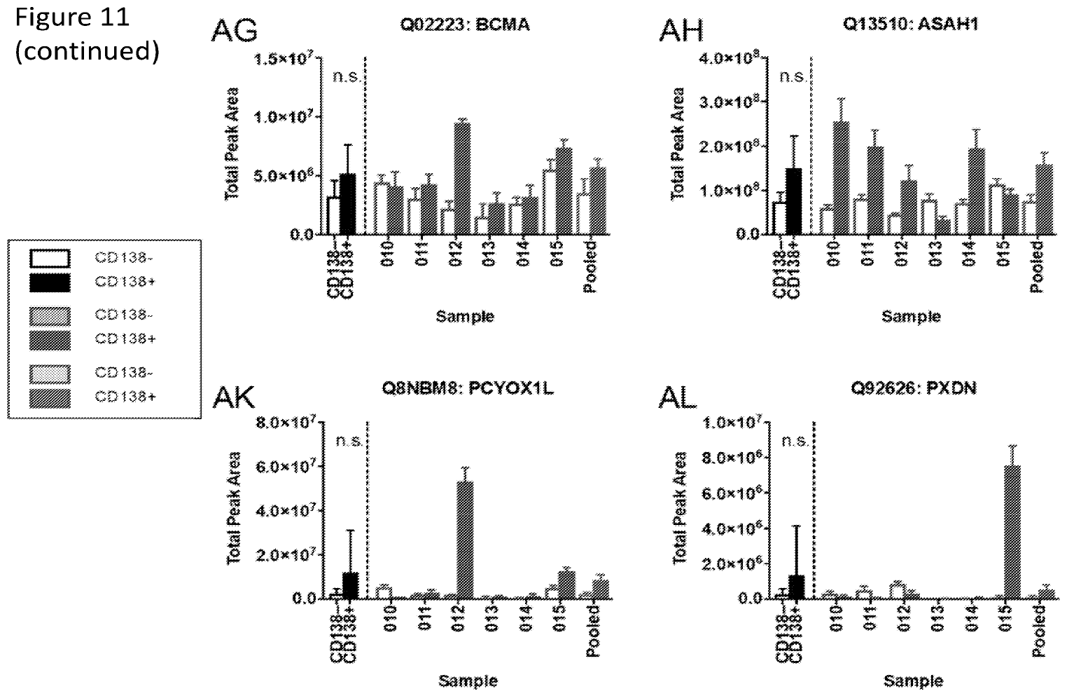

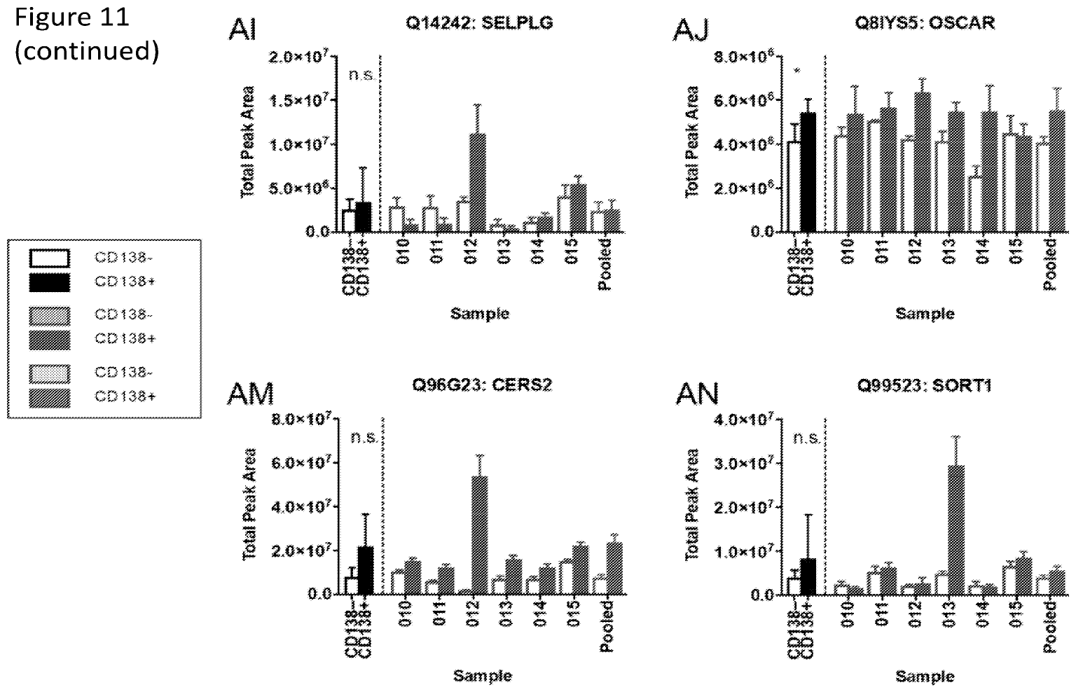

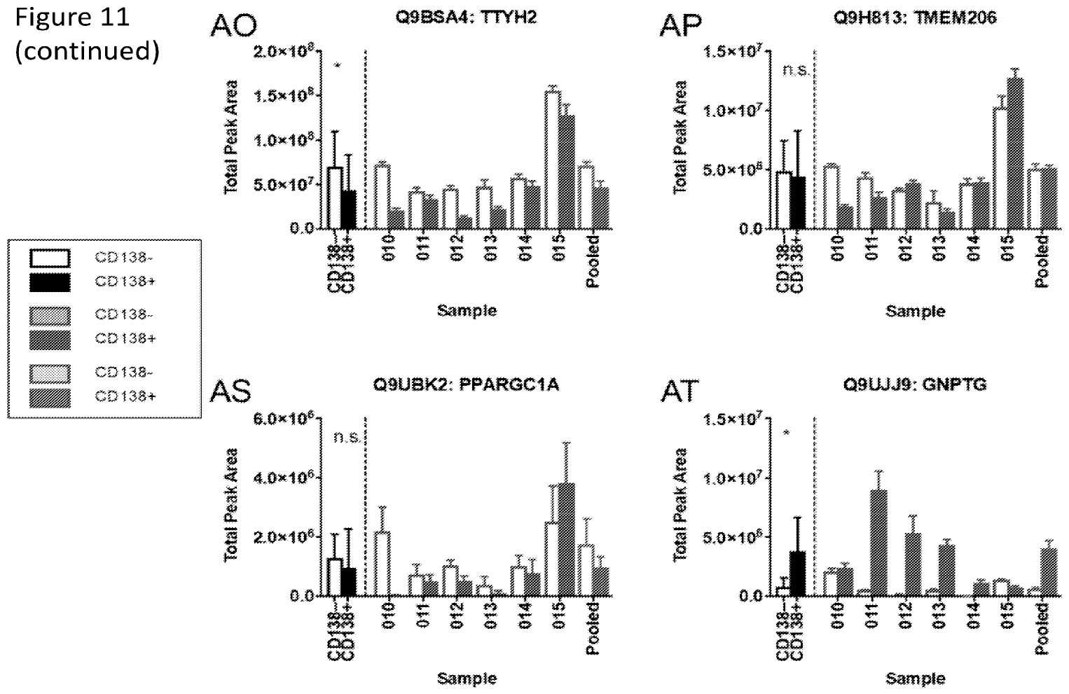

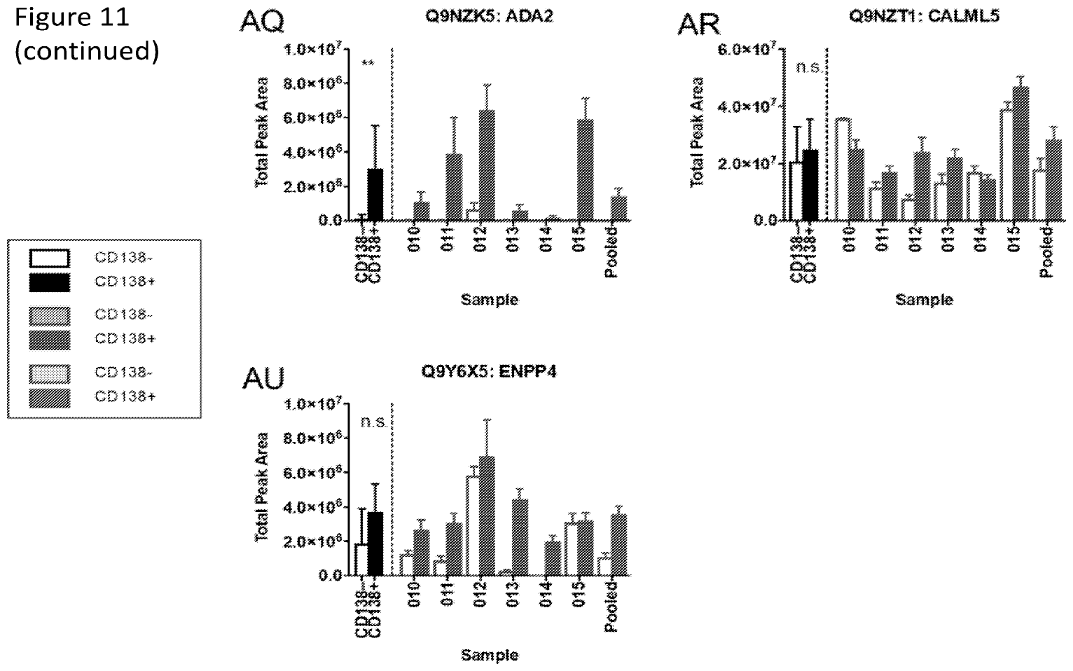

The 73 proteins of interest were assessed by PRM analysis of whole cell lysates of CD138+ and matched CD138− controls. In the first cohort of MMV patient cells (n=6), 59 of the 73 proteins were detected in the primary cell lysates. A number of proteins typically used for immunophenotyping and/or therapy of MM were identified (Table 2). Of the 59 proteins detected in human primary cells, 30 were detected at significantly higher levels in the CD138+ samples (FIG. 5; FIG. 11; Table 3). Many of these proteins are used for diagnosis and prognosis in a range of tumor types (Table 4) and are known to be expressed in malignant hematological and non-hematological cells, as well as in some non-diseased tissues (FIG. 6). There was no association between the abundance of 23 of the proteins and CD138 status (Table 5). Six proteins were detected at lower levels in the CD138+ than the CD138− samples (Table 6), while 14 proteins were not detected in the MM patient cell lysates (Table 7). Failure to detect these proteins in primary cells could be related to differences in proteoforms present (additional modifications or truncations not present in the cell lines) or to differences in expression between cell lines and primary MM samples, among other complications.

TABLE 2
Cell surface N-glycoproteins that are known MM antigens and were selected for detection by PRM

| Protein | Description |
|---|---|
| CD19 | CD19 is a B cell marker that is not typically considered to be a therapeutic target for MM. |
| | However, it may be expressed on a minor MM stem cell subset⁷. CD19 CARs have been used to |
| | treat MM even in the absence of CD19 detection on 99.95% of MM cells⁸. |
| | In our study, CD19 was not detected by M/S on the CD138− or CD138+ samples. |
| CD20 | CD20 is a B cell marker. CD20 expression on MM has been reported in 18% of MM patients⁹. |
| | In our study, CD20 was identified at very low levels across our patient samples, with the exception |
| | of one patient with high CD20 expression in the CD138+ subset. |
| CD27 | No PRM assay developed |
| CD28 | CD28 is a co-stimulatory protein important for T cell activation. Aberrant CD28 expression has been |
| | reported on MM cells from 41% of myeloma patients¹⁰. |
| CD33 | No PRM assay developed |
| CD38 | CD38 was confirmed to have significantly higher expression on the isolated MM cells, supporting the |
| | validity of our approach. |
| CD44 | CD44 is an adhesion molecule with roles in migration and homing. CD44 expression levels |
| | have been associated with MM progression¹¹⁻¹³. |
| | In our study, no significant difference in CD44 expression was present overall. However, one patient in |
| | this study had much higher expression of CD44 on their CD138+ cell subset. |
| CD45 | CD45 is expressed on all nucleated hematopoietic cells. MM cells are reported to have two |
| | distinct populations with low and high CD45 expression^{14,15}. |
| | In our study, CD45 expression was identified as significantly lower on the CD138+ cell population. |
| | However, expression was still detected at some level in cells from all patients, consistent with |
| | previous reports¹⁶⁻¹⁸ |
| CD52 | No PRM assay developed |
| CD54 | CD54 is associated with advanced disease and drug resistance in MM¹⁹⁻²¹. |
| | In our study, CD45 was identified as having significantly higher expression in CD138+ cells. |
| | An anti-CD54 mAb has already completed phase II trials for smoldering MM²². |
| CD56 | CD56 expression is known to be variable on MM cells and may have prognostic significance²³⁻²⁵. |
| | In our study, two of five evaluated patient samples were CD56+ by flow cytometry. However, a PRM |
| | assay for CD56 was not successfully developed. |
| CD177 | No PRM assay developed |
| CD138 | CD138 was confirmed to have significantly higher expression on the isolated MM cells, supporting the |
| | validity of our approach. |
| CD200 | No PRM assay developed |
| BCMA | BCMA is a plasma cell marker that is already an immunotherapy target for MM^{26,27}. |
| | Significant differences in BCMA expression between CD138+ and CD138− samples were not found |
| | in this patient set. |

TABLE 3
Cell surface N-glycoproteins with significantly higher abundance
in the CD138+ MM patient cell subset by PRM analysis

| Uniprot ID | Description | CD protein | Significance |
|---|---|---|---|
| O43852 | Calumenin (CALU) | — | *** |
| O60449 | Lymphocyte antigen 75 (LY75) | CD205 | * |
| P00734 | Prothrombin (F2) | — | ** |
| P01859 | Immunoglobulin heavy constant gamma 2 (IGHG2) | — | ** |
| P02787 | Serotransferrin (TF) | — | * |
| P04180 | Phosphatidylcholine-sterol acyltransferase (LCAT) | — | ** |
| P04216 | Thy-1 membrane glycoprotein | CD90 | ** |
| P05026 | Sodium/potassium-transporting ATPase subunit beta-1 (ATP1B1) | — | ** |
| P05362 | Intercellular adhesion molecule 1 (ICAM1) | CD54 | *** |
| P06127 | T-cell surface glycoprotein CD5 | CD5 | ** |
| P08195 | 4F2 cell-surface antigen heavy chain | CD98hc | ** |
| P13598 | Intercellular adhesion molecule 2 (ICAM2) | — | *** |
| P16615 | Sarcoplasmic/endoplasmic reticulum calcium ATPase 2 (SERCA2, | — | *** |
| | ATP2A2) | | |
| P18827 | Syndecan-1 (SDC1) | CD138 | **** |
| P20020 | Plasma membrane calcium-transporting ATPase 1 (ATP2B1, PMCA1) | — | * |
| P20226 | TATA-box-binding protein (TBP) | — | ** |
| P21796 | Voltage-dependent anion-selective channel protein 1 (VDAC1) | — | ** |
| P26010 | Integrin beta-7 (ITGB7, LPAM-1) | — | ** |
| P28907 | ADP-ribosyl cyclase/cyclic ADP-ribose hydrolase 1 | CD38 | **** |
| P35613 | Basigin (EMMPRIN, BSG) | CD147 | *** |
| P43307 | Translocon-associated protein subunit alpha (SSR1, TRAP-alpha) | — | *** |
| P50851 | Lipopolysaccharide-responsive and beige-like anchor protein (LRBA, | — | *** |
| | CDC4L) | | |
| P52799 | Ephrin-B2 (EFNB2) | — | *** |
| Q00059 | Transcription factor A, mitochondrial (TFAM, mtTFA) | — | ** |
| Q13740 | CD166 antigen (ALCAM, MEMD, CD6L) | CD166 | ** |
| Q8IYS5 | Osteoclast-associated immunoglobulin-like receptor (OSCAR) | — | * |
| Q96KA5 | Cleft lip and palate transmembrane protein 1-like protein (CLPTM1L) | — | **** |
| Q9NSC5 | Homer protein homolog 3 (Homer3) | — | ** |
| Q9NZK5 | Adenosine deaminase 2 (ADA2) | — | ** |
| Q9UJJ9 | N-acetylglucosamine-1-phosphotransferase subunit gamma (GNPTG) | — | * |

* for p < 0.05,
** for p < 0.01,
*** for p < 0.001,
**** for p < 0.0001

TABLE 4
Cell surface N-glycoproteins identified by PRM on primary MM
cells with potential diagnostic or prognostic significance.

| Protein | Prognostic Significance | Reference |
|---|---|---|
| CD90 | Associated with tumorigenesis and poor survival in hepatocellular carcinoma, | (28-32) |
| | hepatoblastoma, and lung cancer | |
| ATP1B1 | In cytogenetically normal AML, this protein is associated with shorter overall | (33) |
| | survival | |
| CD5 | Identified as a poor prognostic marker for cancers such as diffuse large B-cell | (34, 35) |
| | lymphoma and mantle cell lymphoma | |
| CD98HC | Associated with poor survival in cancers such as oropharyngeal cancer, | (36-38) |
| | hypopharyngeal squamous cell carcinoma, and gastric cancer | |
| ICAM2 | Increased expression has been associated with poor survival in various cancers; | (39, 40) |
| | may be associated with anti-tumor immune response | |
| SERCA2 | High expression has been associated with tumor grade and metastasis in | (41, 42) |
| (ATP2A2) | colorectal cancer; expression has been associated with response to bortezomib in | |
| | liposarcoma | |
| TBP | Known to be upregulated by oncogenic signaling pathways; may play an early | (43) |
| | role in tumorigenesis of cancers such as colon carcinomas and adenomas | |
| LRBA | Shown to be predictive of mortality and recurrence in breast cancer | (44) |
| EFNB2 | Significant correlations between expression, overall survival, and disease-free | (45-48) |
| | survival have been noted in various solid tumors | |
| HOMER3 | Overexpression is significantly associated with advanced stage in esophageal | (49) |
| | squamous cell carcinoma | |
| ADA2 | Increased expression has been correlated with lymph node involvement, grade, | (50) |
| | and tumor size in breast cancer | |
| CLPTM1L | Overexpression has been associated with poor prognosis in lung cancer; | (51, 52) |
| | demonstrated to play a role in cisplatin resistance | |
| CD166 | Plays a significant role in MM progression and BM homing; strongly correlated | (53, 54) |
| | with unfavorable prognosis in melanoma | |

TABLE 5
Proteins with no significant difference in abundance
in the CD138+ cell subset as determined by PRM

| Uniprot | | CD | Detected by |
|---|---|---|---|
| ID | Description | protein | PRM? |
| O60266 | Adenylate cyclase type 3 (ADCY3) | — | Yes |
| P01033 | Metalloproteinase inhibitor 1 (TIMP1) | — | Yes |
| P02786 | Transferrin receptor protein 1 (TFR1) | CD71 | Yes |
| P05023 | Sodium/potassium-transporting ATPase subunit alpha-1 | — | Yes |
| | (ATP1A1) | | |
| P11836 | B-lymphocyte antigen CD20 | CD20 | Yes |
| P12259 | Coagulation factor V (F5) | — | Yes |
| P16070 | CD44 antigen | CD44 | Yes |
| P23634 | Plasma membrane calcium-transporting ATPase 4 (ATP2B4) | — | Yes |
| P32942 | Intercellular adhesion molecule 3 (ICAM3) | — | Yes |
| P54709 | Sodium/potassium-transporting ATPase subunit beta-3 | — | Yes |
| | (ATP1B3) | | |
| P55083 | Microfibril-associated glycoprotein 4 (MFAP4) | — | Yes |
| P84243 | Histone H3.3 (H3F3A) | — | Yes |
| Q02223 | Tumor necrosis factor receptor superfamily member 17 (BCMA, | — | Yes |
| | TNFRSF17) | | |
| Q13510 | Acid ceramidase (ASAH1) | — | Yes |
| Q14242 | P-selectin glycoprotein ligand 1 (SELPLG) | — | Yes |
| Q8NBM8 | Prenylcysteine oxidase-like (PCYOX1L) | — | Yes |
| Q92626 | Peroxidasin homolog (PXDN) | — | Yes |
| Q96G23 | Ceramide synthase 2 (CERS2) | — | Yes |
| Q99523 | Sortilin (SORT1) | — | Yes |
| Q9H813 | Transmembrane protein 206 (TMEM206) | — | Yes |
| Q9NZT1 | Calmodulin-like protein 5 (CALML5) | — | Yes |
| Q9UBK2 | Peroxisome proliferator-activated receptor gamma coactivator 1- | — | Yes |
| | alpha (PPARGC1A) | | |
| Q9Y6X5 | Bis(5′-adenosyl)-triphosphatase ENPP4 | — | Yes |

TABLE 6
Proteins with significantly lower abundance in
the CD138+ cell subset as determined by PRM.

| Uniprot | | CD | Detected by |
|---|---|---|---|
| ID | Description | protein | PRM? |
| P04234 | T-cell surface glycoprotein CD3 delta chain | CD3D | Yes |
| P05107 | Integrin beta-2 (ITGB2) | CD18 | Yes |
| P08575 | Receptor-type tyrosine-protein phosphatase C (PTPRC) | CD45 | Yes |
| P11049 | Leukocyte antigen CD37 | CD37 | Yes |
| P11279 | Lysosome-associated membrane glycoprotein 1 (LAMP1) | CD107a | Yes |
| Q9BSA4 | Protein tweety homolog 2 (TTYH2) | — | Yes |

TABLE 7
Proteins not detected in patient MM samples by PRM

| Uniprot | | CD | Detected by |
|---|---|---|---|
| ID | Description | protein | PRM? |
| O43278 | Kunitz-type protease inhibitor 1 (SPINT1) | — | No |
| O75629 | Protein CREG1 | — | No |
| P10747 | T-cell-specific surface glycoprotein CD28 | CD28 | No |
| P15391 | B-lymphocyte antigen CD19 | CD19 | No |
| P30203 | T-cell differentiation antigen CD6 | CD6 | No |
| P32004 | Neural cell adhesion molecule L1 (L1CAM) | CD171 | No |
| P33527 | Multidrug resistance-associated protein 1 (ABCC1) | — | No |
| P36888 | Receptor-type tyrosine-protein kinase FLT3 | CD135 | No |
| P43121 | Cell surface glycoprotein MUC18 (MCAM) | — | No |
| Q13201 | Multimerin-1 (MMRN1) | — | No |
| Q5QGZ9 | C-type lectin domain family 12 member A (CLEC12A) | — | No |
| Q96DU3 | SLAM family member 6 (SLAMF6) | — | No |
| Q9H7F0 | Probable cation-transporting ATPase 13A3 (ATP13A3) | — | No |
| Q9HA82 | Ceramide synthase 4 (CERS4) | — | No |

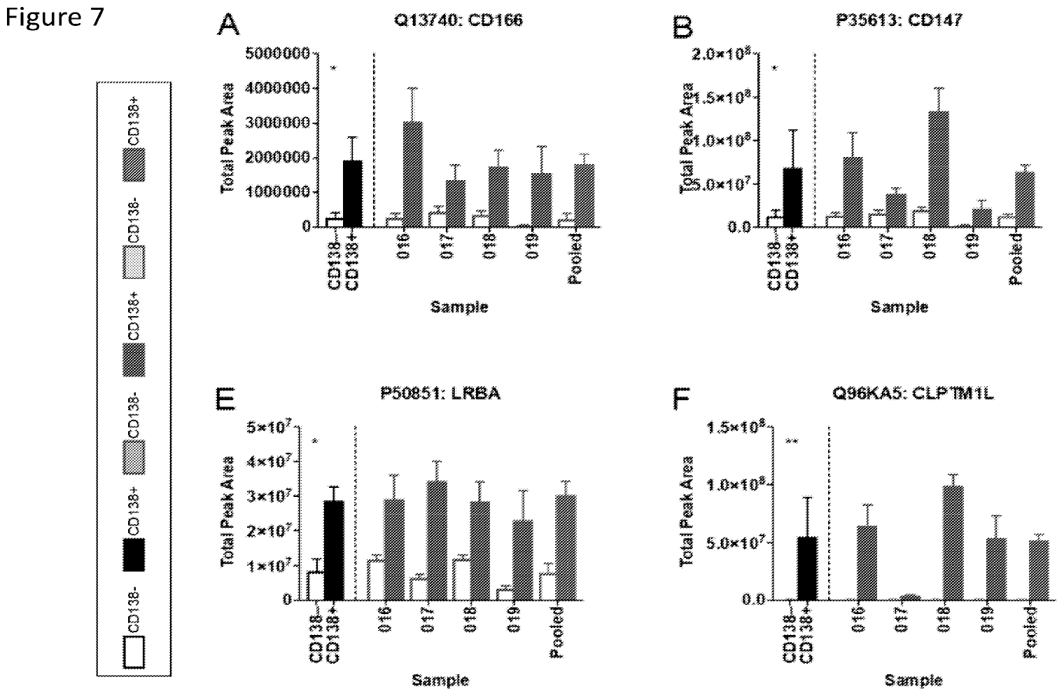

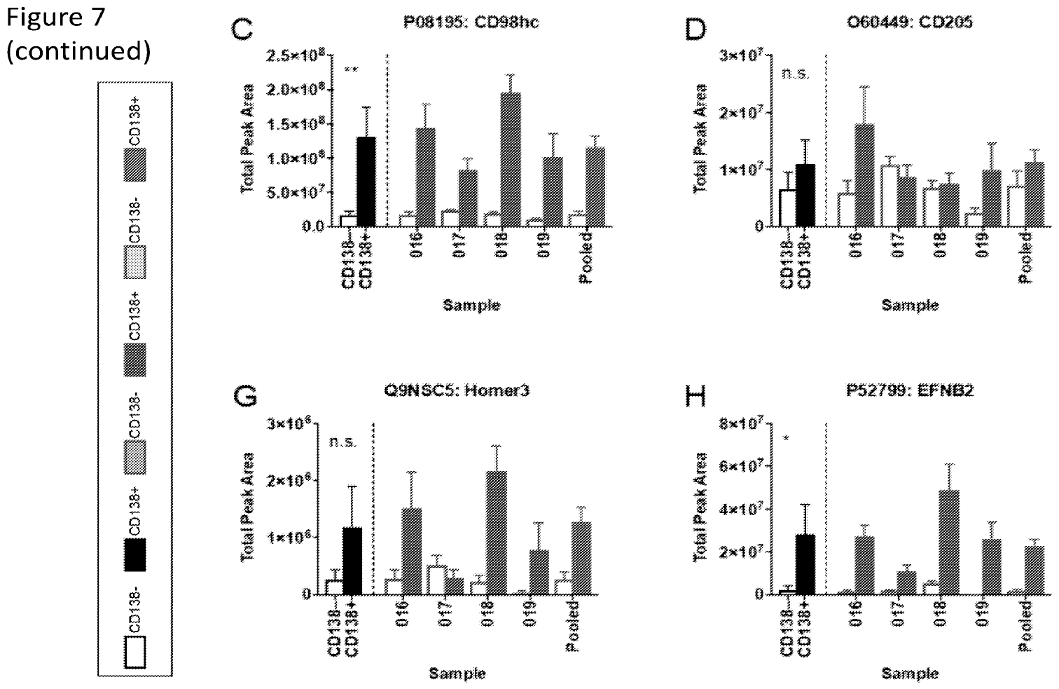

Based on an analysis of published literature and the reported expression in other cell types throughout the body, we narrowed our interest to 9 potential therapeutic targets for MM. Five of the selected proteins (i.e., CD5, CD166 (also known as ALCAMV), CD147 (also known as Basigin or EMMIPRIN), CD98hc (also known as 4F2 cell-surface antigen heavy chain), and CD205), are currently under investigation as therapeutic targets in other cancer types. An additional four proteins (i.e., LRBA, CLPTMIL, Homer3, and EFNB2), have not been previously reported as therapeutic targets in any malignancy. In a PRMV analysis of a second independent cohort of MIV patient samples, six of these proteins (i.e., LRBA, CLPTM1L, EFNB2, CD166, CD147, and CD98hc) were confirmed to be significantly more abundant in the CD138+ cell subset (FIG. 7). Two proteins (Homer3 and CD205) were present at higher levels but their abundance did not reach statistical significance, likely due to the small sample size (n=4). CD5 was not detected in this second analysis. Thus, our results for this protein are inconclusive.

### Validation of Therapeutic Targets on Live Cells

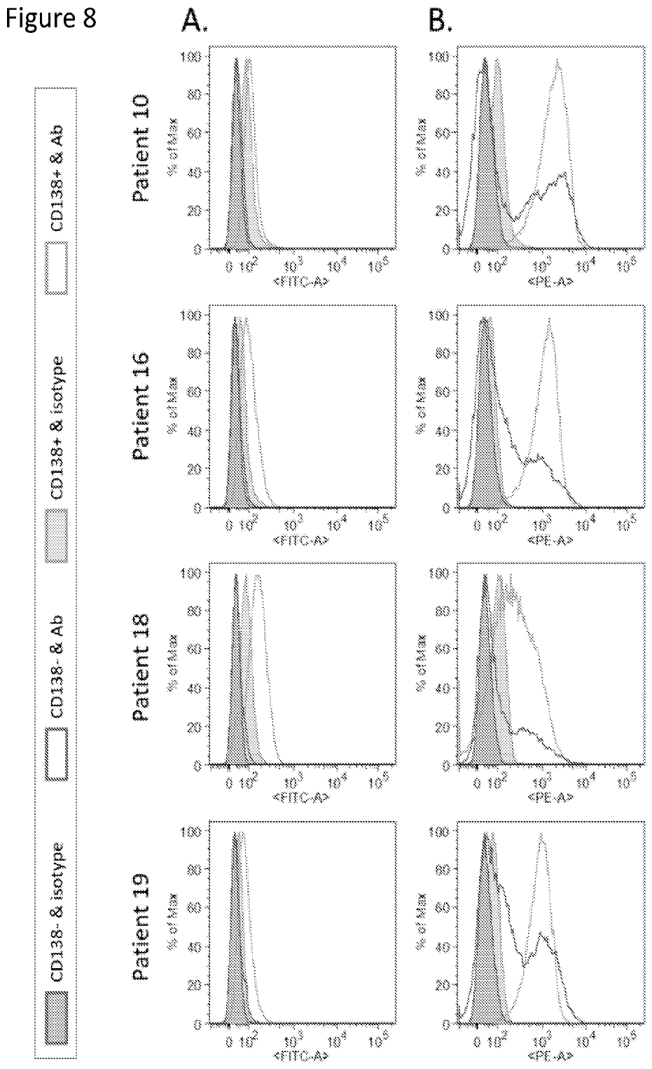

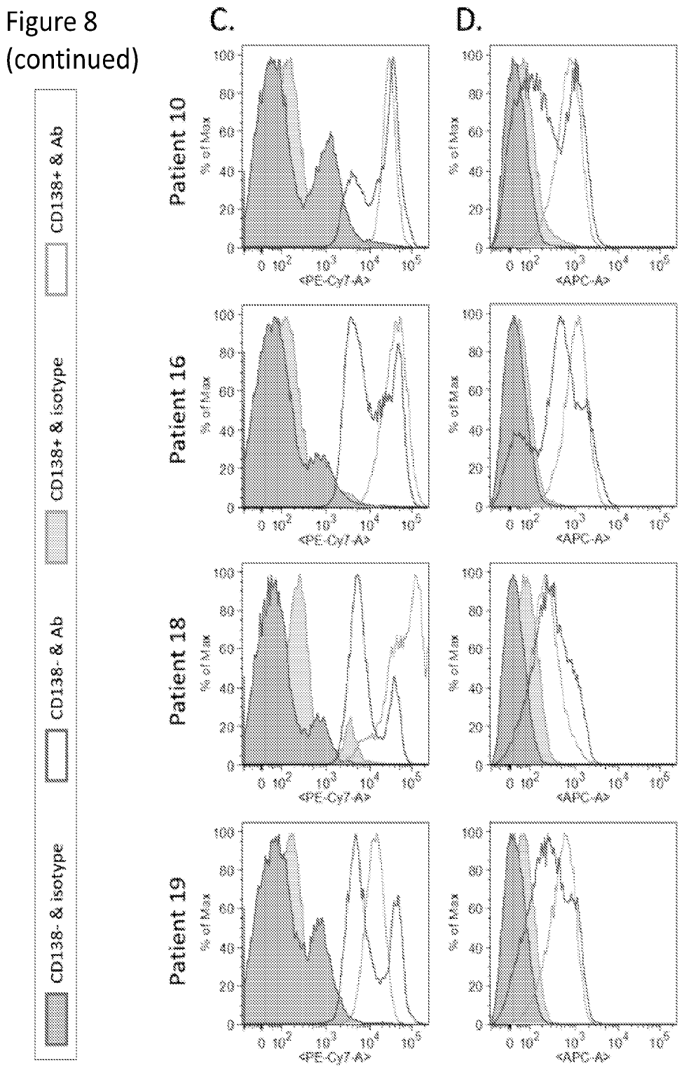

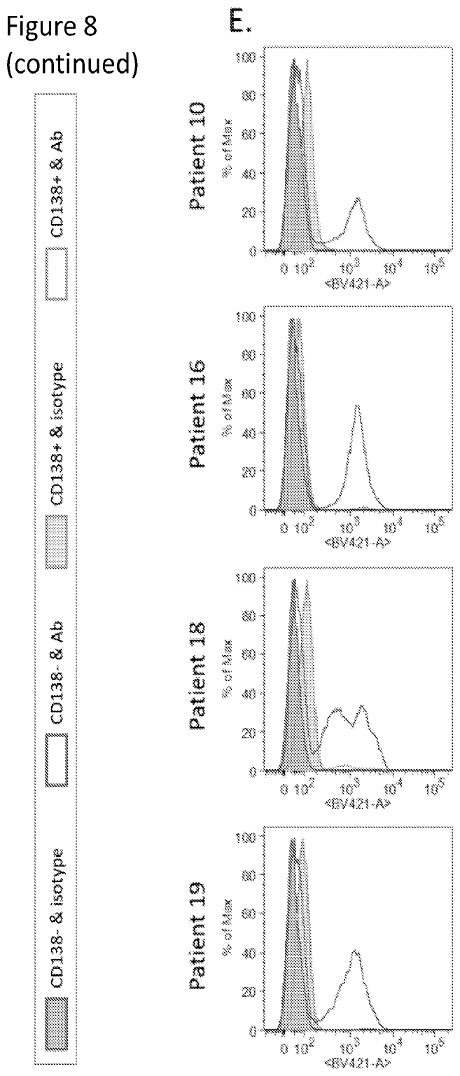

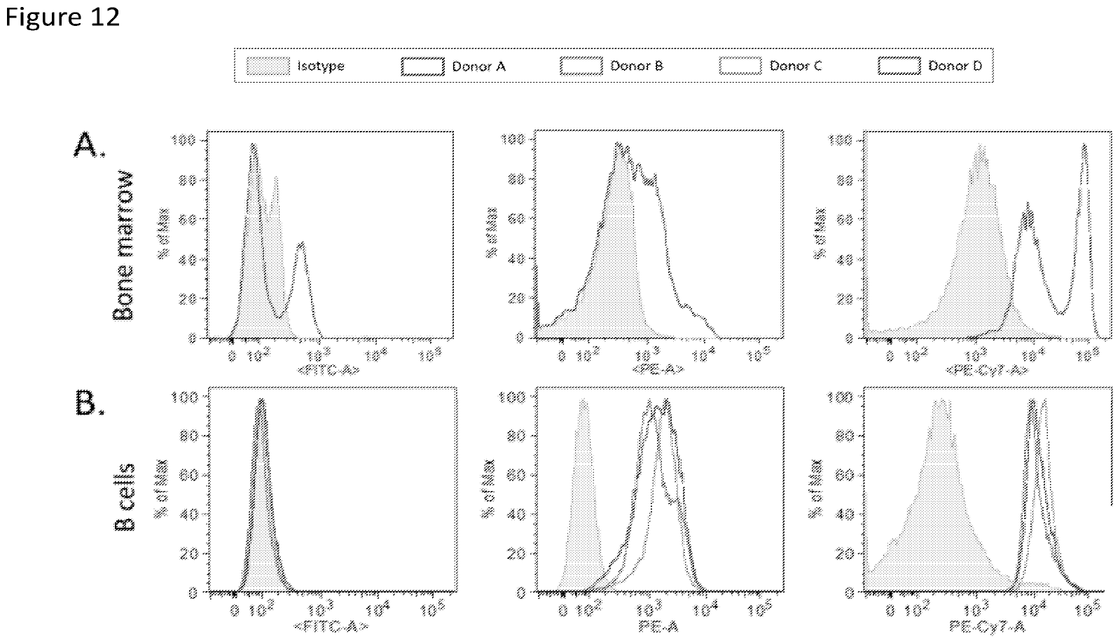

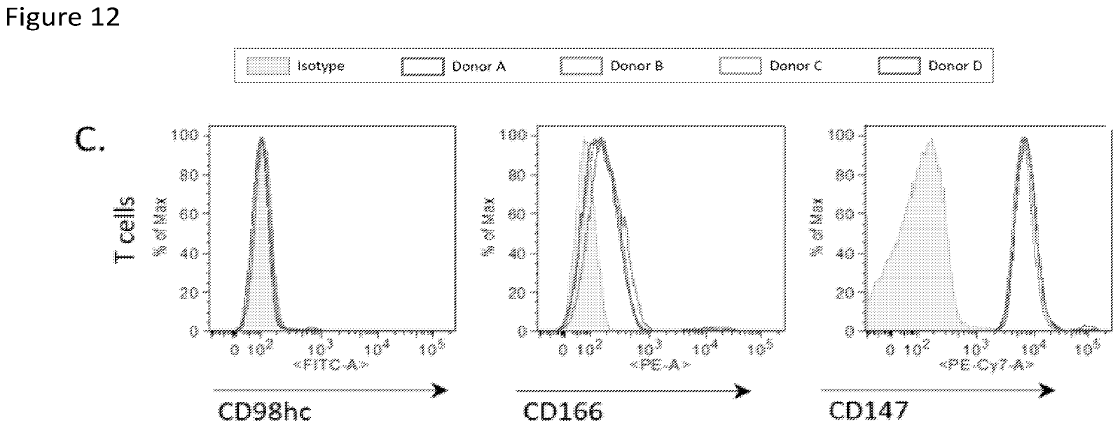

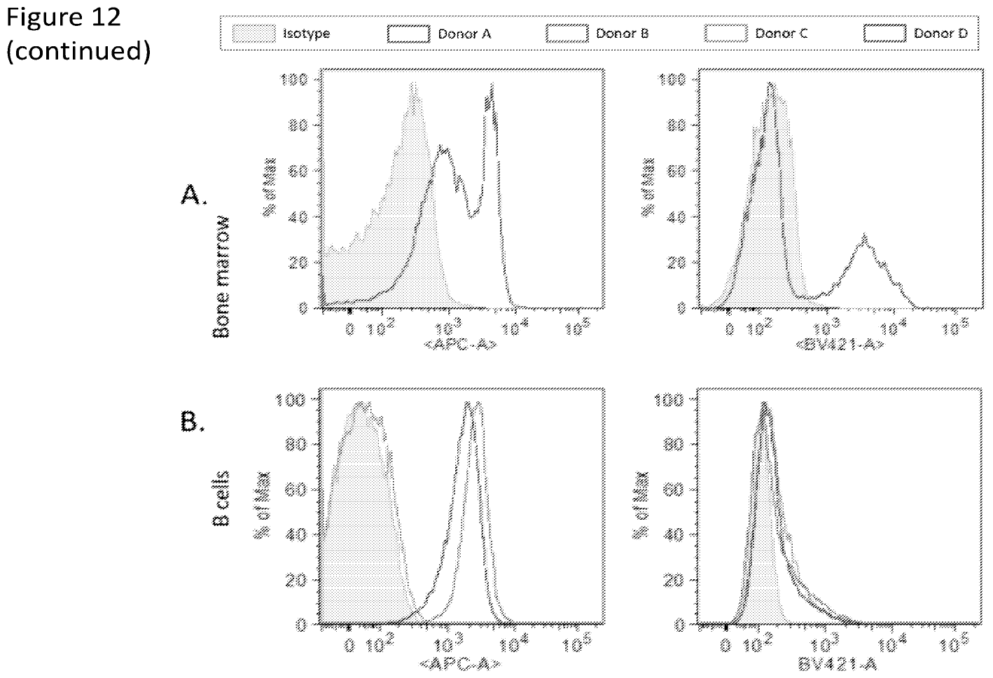

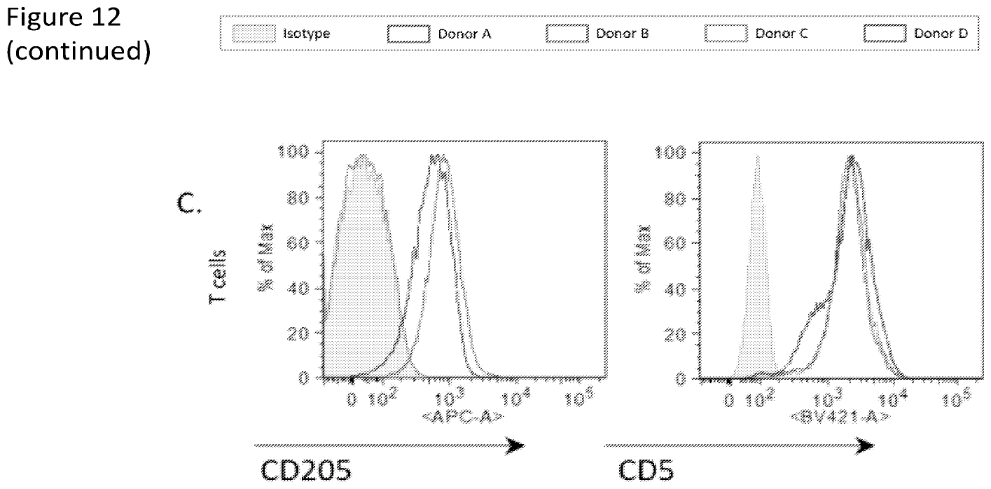

Final validation regarding cell surface expression of candidate proteins was performed using FCM on CD138+ and CD138− MM patient cells. For the nine candidate proteins of interest, 5 corresponding monoclonal antibodies were identified that were suitable for FCM: anti-CD5, anti-CD98hc, anti-CD147, anti-CD166, and anti-CD205. Analysis of CD138+ patient samples revealed the presence of all five antigens with some variation in expression levels (FIG. 8). Expression of CD5 was very low overall, present on 0.01%-5.7% of CD138+ cells, while CD147 and CD166 were expressed on nearly all CD138+ cells in most MM patient specimens examined (Table 8). MM patient-dependent variations in expression were also observed for CD98hc (1.0%-17.3% of CD38+ cells) and CD205 (25.1%-94.0% of CD138+ cells). Analysis of normal BMNCs, T cells, and B cells was included as a control; inter-donor variation in FCM assays was also observed in these cell types (FIG. 12). Altogether, these FCM data provide orthogonal confirmation that the five candidate proteins of interest originally detected by MS are present on the cell surface of human primary MM cells. Monoclonal antibodies are not yet available for LRBA, CLPTM1L, EFNB2, and Homer3, precluding their validation by FCM at this time.

TABLE 8
Percent expression of MM antigens
on CD138+ patient cells by FCM

| Antigen | Patient 10 | Patient 16 | Patient 18 | Patient 19 |
|---|---|---|---|---|
| CD5 | 0.01% | 1.92% | 5.63% | 1.07% |
| CD98hc | 1.0% | 5.2% | 17.3% | 12.7% |
| CD147 | 99.9% | 99.9% | 96.3% | 99.8% |
| CD166 | 95.3% | 97.8% | 51.3% | 99.0% |
| CD205 | 75.2% | 94.0% | 25.1% | 79.8% |

### Discussion:

In this study, we report a description of the MM cell surface N-glycoproteome based on CSC analysis of MM cell lines, followed by PRM and FCM validation of selected protein candidates in primary cells isolated from MM patient BM. While several MS-based studies of MM have been undertaken previously(16-19), our study offers several advantages, including the use of CSC to specifically detect proteins present on the cell surface, the use of multiple cell lines to account for possible differences among patients, and the use of both PRM and FCM to determine if proteins of interest discovered on immortalized cell lines are relevant to primary human MM cells.

PRM analyses identified 30 proteins that are significantly higher in abundance in the CD138+ cells from MM patients than in the CD138− cells from these patients. Although B cells or CD138+ cells from the BM of non-MM patients would be a more informative sample for comparison, BM samples from healthy individuals are challenging to obtain, and were not available for the present study. Our dataset of proteins with significantly higher abundance in the CD138+ subsets of cells includes known MM antigens, as well as proteins that may be involved in various MM pathologies including bone dysfunction, development and growth, metastasis and invasion, and therapy resistance.

Many of the proteins identified in this study that are more abundant in MM cells than in control cells (e.g., CD90, ATP1B1, CD5, CD98, ICAM-2, SERCA2, TBP, LRBA, EFNB2, Homer3, ADA2, CLPTM1L, and CD166) have been linked with poor prognoses in other cancer types. Proteins that are already associated with progression, poor prognosis, or drug resistance in MM include ICAM-1(27), VDAC1(28, 29), ITGB7(30, 31), CD147(32-34), and TFAM(35). To our knowledge, calumenin, CD205, SSR1 (also known as TRAP-alpha), and GNPTG have not previously been described as diagnostic or prognostic indicators for any cancer type. Based on our results, however, they may be relevant markers for MM. Prothrombin (F2) and serotransferrin (TF) were also identified as highly abundant in our CD138+ subset, however, the universal expression of these proteins may make their development as therapeutic targets for MM unsuitable.

In MM patients, calcium levels are often dysregulated and elevated. For instance, hypercalcemia, a result of osteoclastic bone resorption and release of calcium into the extracellular fluid, is frequently observed in MM patients with a large tumor volume. Consistent with this association, various calcium-related proteins were identified in the CD138+ patient samples, including calumenin, SERCA2 (also known as ATP2A2), ATP2B1 (also known as PMCA1), and Homer3. Several proteins with known roles in BM homing and bone disease were also identified, such as TF, ITGB7, CD147, EFNB2, and CD166. Identification of known MM-linked proteins, and proteins involved in MM pathologies, supports the credibility and utility of this approach.

Unsurprisingly, differences in protein abundance levels among patient samples were observed. Several of these proteins are associated with roles in cancer migration and invasion, such as CD90 (also known as Thy-1)(36, 37), CD147(38, 39), and Homer3.(40, 41) Some are associated with growth/tumorigenesis, such as CD98(42-44), SERCA2(45), LRBA(46), and CD166.(47, 48) Proteins involved in resistance to apoptosis and therapy were also found to differ among the 10 MM patients. These variations may be related to MM stage, aggressiveness, or responsiveness to therapy, among other factors. Larger patient cohorts will be necessary in order to validate the diagnostic or prognostic impact of these findings.

We have identified nine promising MM immunotherapy targets that were originally detected in MM cell lines and subsequently validated in primary MM cells (i.e., CD5, CD166, CD147, CD98h, CD205, LRBA, CLPTM1L, Homer3, and EFNB2). All nine targets have been validated by PRM analysis of protein abundance at the whole cell level. Five target proteins have been further validated by FCM analysis of their abundance specifically at the surface of MM patient cells. While reported expression of some of these antigens on subsets of normal cell types does not preclude their use as immunotherapy targets, it does warrant caution in their development. Indeed, five of these nine targets, including CD5, CD147, CD205, CD98hc, and CD166, are already under investigation for other tumor types. The therapies developed in the context of those tumor types may therefore be subsequently tested for use in MM. This is important because safety information (including potential ‘on-target, off-tumor’ effects) established in those studies may inform their application to MM. For example, a phase 1 clinical trial studying CD5 CARs for T-cell leukemia or lymphoma is currently underway (NCT03081910). A radio-immunotherapy product targeting CD147 has been evaluated in clinical trials for hepatocellular carcinoma.(49) There is also an antibody-drug conjugate against CD205 (also known as LY75) currently under phase 1 clinical trial investigation for non-Hodgkin lymphoma (NCT03403725). A phase 1 clinical trial was recently completed for a mAb recognizing CD98 to treat relapsed or refractory AML (NCT02040506). In a phase 1/2 study for selected solid tumors (NCT03149549), an antibody-drug conjugate targeting CD166 is currently being evaluated.

Beyond the well-known proteins identified in our study, additional proteins of interest have been described in MM or other cancers but, to our knowledge, have yet to be tested clinically as immunotherapy targets. This includes LRBA(46), EFNB2(50), and CLPTM1L(51, 52). Also, Homer3 has not previously been identified as a therapeutic target; however, interestingly, anti-Homer3 antibodies have been found in MM patients with complete response to donor lymphocyte infusion(53), suggesting that this protein may also be a promising antigen for immunotherapy. It is expected that when suitable monoclonal antibodies are available for LRBA, CLPTM1L, EFNB2 and Homer3, similar FCM-based validation efforts will be possible for these targets.

In addition to providing a detailed view of the MM cell surface and identifying new therapeutic targets, we have developed PRM assays that may be applied to patient biopsies for diagnostic or disease monitoring purposes. Currently, MM monitoring is carried out using techniques such as assessment of paraprotein levels and quantification of percentages of plasma cells in the BM. However, more informative readouts that are quick, accurate, and sensitive would benefit the care of MM patients. This is especially relevant given the heterogeneity of MM, the continued development of new therapies, and the need for individual patient analyses when administering targeted immunotherapies. In the long-term, it is possible that MM patients could be efficiently screened using an MS-based assay to inform selection of an appropriate personalized therapy, as well as to track response or resistance to treatment over time. To support this effort, the PRM assays developed here are freely available in Panorama.

### Conclusions:

This study contributes to knowledge and understanding of the MM cell surface and provides a rich resource to inform future studies aimed at characterizing malignancy. We have identified nine proteins that may be relevant, novel MM immunotherapy targets, as well as multiple proteins of prognostic and/or biologic interest. Further clinical validation of these novel MM targets and assays will expand the ability to diagnose, monitor, and treat this disease, with the goal of improving patient outcomes and quality of life.

### REFERENCES

1. Lohr J G, Stojanov P, Carter S L, Cruz-Gordillo P, Lawrence M S, Auclair D, et al. Widespread genetic heterogeneity in multiple myeloma: implications for targeted therapy. Cancer Cell. 2014; 25(1):91-101.
2. Landgren O, Rajkumar S V. New Developments in Diagnosis, Prognosis, and Assessment of Response in Multiple Myeloma. Clinical cancer research: an official journal of the American Association for Cancer Research. 2016; 22(22):5428-33.
3. Flores-Montero J, de Tute R, Paiva B, Perez J J, Bottcher S, Wind H, et al. Immunophenotype of normal vs. myeloma plasma cells: Toward antibody panel specifications for MRD detection in multiple myeloma. Cytometry B Clin Cytom. 2016; 90(1):61-72.
4. Levin A, Hari P, Dhakal B. Novel biomarkers in multiple myeloma. Transl Res. 2018; 201:49-59.
5. Flanders A, Stetler-Stevenson M, Landgren O. Minimal residual disease testing in multiple myeloma by flow cytometry: major heterogeneity. Blood. 2013; 122(6):1088-9.
6. Rajkumar S V. Multiple myeloma: 2016 update on diagnosis, risk-stratification, and management. American journal of hematology. 2016; 91(7):719-34.
7. Cancer Stat Facts: Myeloma: National Cancer Institute Surveillance, Epidemiology, and End Results Program; 2014 [Available from: seer.cancer.gov/statfacts/html/mulmy.html.
8. Shah V, Sherborne A L, Walker B A, Johnson D C, Boyle E M, Ellis S, et al. Prediction of outcome in newly diagnosed myeloma: a meta-analysis of the molecular profiles of 1905 trial patients. Leukemia. 2018; 32(1):102-10.
9. Lagana A, Perumal D, Melnekoff D, Readhead B, Kidd B A, Leshchenko V, et al. Integrative network analysis identifies novel drivers of pathogenesis and progression in newly diagnosed multiple myeloma. Leukemia. 2018; 32(1):120-30.
10. Perna F, Berman S H, Soni R K, Mansilla-Soto J, Eyquem J, Hamieh M, et al. Integrating Proteomics and Transcriptomics for Systematic Combinatorial Chimeric Antigen Receptor Therapy of AML. Cancer Cell. 2017; 32(4):506-19 e5.
11. Mirkowska P, Hofmann A, Sedek L, Slamova L, Mejstrikova E, Szczepanski T, et al. Leukemia surfaceome analysis reveals new disease-associated features. Blood. 2013; 121(25):e149-59.
12. Hofmann A, Thiesler T, Gerrits B, Behnke S, Sobotzki N, Omasits U, et al. Surfaceome of classical Hodgkin and non-Hodgkin lymphoma. Proteomics Clin Appl. 2015; 9(7-8):661-70.
13. Lee J K, Bangayan N J, Chai T, Smith B A, Pariva T E, Yun S, et al. Systemic surfaceome profiling identifies target antigens for immune-based therapy in subtypes of advanced prostate cancer. Proceedings of the National Academy of Sciences of the United States of America. 2018; 115(19):E4473-E82.
14. Gundry R L, Riordon D R, Tarasova Y, Chuppa S, Bhattacharya S, Juhasz O, et al. A cell surfaceome map for immunophenotyping and sorting pluripotent stem cells. Mol Cell Proteomics. 2012; 11(8):303-16.
15. Bausch-Fluck D, Hofmann A, Bock T, Frei A P, Cerciello F, Jacobs A, et al. A mass spectrometric-derived cell surface protein atlas. PLoS One. 2015; 10(3):e0121314.
16. Choksawangkarn W, Edwards N, Wang Y, Gutierrez P, Fenselau C. Comparative study of workflows optimized for in-gel, in-solution, and on-filter proteolysis in the analysis of plasma membrane proteins. J Proteome Res. 2012; 11(5):3030-4.
17. Choksawangkarn W, Kim S K, Cannon J R, Edwards N J, Lee S B, Fenselau C. Enrichment of plasma membrane proteins using nanoparticle pellicles: comparison between silica and higher density nanoparticles. J Proteome Res. 2013; 12(3):1134-41.
18. Dytfeld D, Rosebeck S, Kandarpa M, Mayampurath A, Mellacheruvu D, Alonge M M, et al. Proteomic profiling of naive multiple myeloma patient plasma cells identifies pathways associated with favourable response to bortezomib-based treatment regimens. British journal of haematology. 2015; 170(1):66-79.
19. Fernando R C, de Carvalho F, Mazzotti D R, Evangelista A F, Braga W M T, de Lourdes Chauffaille M, et al. Multiple myeloma cell lines and primary tumors proteoma: protein biosynthesis and immune system as potential therapeutic targets. Genes Cancer. 2015; 6(11-12):462-71.
20. Wollscheid B, Bausch-Fluck D, Henderson C, O'Brien R, Bibel M, Schiess R, et al. Mass-spectrometric identification and relative quantification of N-linked cell surface glycoproteins. Nature biotechnology. 2009; 27(4):378-86.
21. Boheler K R, Bhattacharya S, Kropp E M, Chuppa S, Riordon D R, Bausch-Fluck D, et al. A human pluripotent stem cell surface N-glycoproteome resource reveals markers, extracellular epitopes, and drug targets. Stem cell reports. 2014; 3(1):185-203.
22. Fujinaka C M, Waas M, Gundry R L. Mass Spectrometry-Based Identification of Extracellular Domains of Cell Surface N-Glycoproteins: Defining the Accessible Surfaceome for Immunophenotyping Stem Cells and Their Derivatives. Methods Mol Biol. 2018; 1722:57-78.
23. Waas M, Pereckas M, Jones Lipinski R A, Ashwood C, Gundry R L. SP2: Rapid and Automatable Contaminant Removal from Peptide Samples for Proteomic Analyses. J Proteome Res. 2019; 18(4):1644-56.
24. MacLean B, Tomazela D M, Shulman N, Chambers M, Finney G L, Frewen B, et al. Skyline: an open source document editor for creating and analyzing targeted proteomics experiments. Bioinformatics. 2010; 26(7):966-8.
25. Uhlen M, Fagerberg L, Hallstrom B M, Lindskog C, Oksvold P, Mardinoglu A, et al. Proteomics. Tissue-based map of the human proteome. Science. 2015; 347(6220):1260419.
26. Laabi Y, Gras M P, Carbonnel F, Brouet J C, Berger R, Larsen C J, et al. A new gene, BCM, on chromosome 16 is fused to the interleukin 2 gene by a t(4; 16)(q26;p13) translocation in a malignant T cell lymphoma. EMBO J. 1992; 11(11):3897-904.
27. Sampaio M S, Vettore A L, Yamamoto M, Chauffaille Mde L, Zago M A, Colleoni G W. Expression of eight genes of nuclear factor-kappa B pathway in multiple myeloma using bone marrow aspirates obtained at diagnosis. Histol Histopathol. 2009; 24(8):991-7.
28. Grills C, Jithesh P V, Blayney J, Zhang S D, Fennell D A. Gene expression meta-analysis identifies VDAC1 as a predictor of poor outcome in early stage non-small cell lung cancer. PLoS One. 2011; 6(1):e14635.
29. Liu S, Ishikawa H, Tsuyama N, Li F J, Abroun S, Otsuyama K I, et al. Increased susceptibility to apoptosis in CD45(+) myeloma cells accompanied by the increased expression of VDAC1. Oncogene. 2006; 25(3):419-29.
30. Neri P, Ren L, Azab A K, Brentnall M, Gratton K, Klimowicz A C, et al. Integrin beta7-mediated regulation of multiple myeloma cell adhesion, migration, and invasion. Blood. 2011; 117(23):6202-13.
31. Choudhury S R, Ashby C, Tytarenko R, Wang Y, Patel P H, Mikulasova A, et al. Intragenic DNA-hypomethylation promotes overexpression of ITGB7 in MF subgroup of multiple myeloma. American Association for Cancer Research Proceedings. 2018; 78(13 Supplement):5324.
32. Wang Y, Fan R, Lei L, Wang A, Wang X, Ying S, et al. Interleukin-6 Drives Multiple Myeloma Progression through Upregulating of CD147/Emmprin Expression and Its Sialylation. Blood. 2017; 130(Suppl 1):3037.
33. Zhu D, Wang Z, Zhao J J, Calimeri T, Meng J, Hideshima T, et al. The Cyclophilin A-CD147 complex promotes the proliferation and homing of multiple myeloma cells. Nature medicine. 2015; 21(6):572-80.
34. Lacina P, Butrym A, Mazur G, Bogunia-Kubik K. BSG and MCT1 Genetic Variants Influence Survival in Multiple Myeloma Patients. Genes (Basel). 2018; 9(5).
35. Ruiz-Heredia Y, Samur M K, Ortiz-Ruiz A, Fernindez R A, Sanchez-Vega B, Blazquez A, et al. Abnormalities in Mitochondrial DNA Copy Number Have Pathogenetic and Prognostic Implications in Multiple Myeloma. Blood. 2017; 130(Suppl 1):4378.
36. Avril T, Etcheverry A, Pineau R, Obacz J, Jegou G, Jouan F, et al. CD90 Expression Controls Migration and Predicts Dasatinib Response in Glioblastoma. Clinical cancer research: an official journal of the American Association for Cancer Research. 2017; 23(23):7360-74.
37. Zhang K, Che S, Su Z, Zheng S, Zhang H, Yang S, et al. CD90 promotes cell migration, viability and sphereforming ability of hepatocellular carcinoma cells. Int J Mol Med. 2018; 41(2):946-54.
38. Xiao W, Zhao S, Shen F, Liang J, Chen J. Overexpression of CD147 is associated with poor prognosis, tumor cell migration and ERK signaling pathway activation in hepatocellular carcinoma. Exp Ther Med. 2017; 14(3):2637-42.
39. Yang S, Qi F, Tang C, Wang H, Qin H, Li X, et al. CD147 promotes the proliferation, invasiveness, migration and angiogenesis of human lung carcinoma cells. Oncol Lett. 2017; 13(2):898-904.
40. Chen S, Zhang Y, Wang H, Zeng Y Y, Li Z, Li M L, et al. WW domain-binding protein 2 acts as an oncogene by modulating the activity of the glycolytic enzyme ENO1 in glioma. Cell Death Dis. 2018; 9(3):347.
41. Wu J, Pipathsouk A, Keizer-Gunnink A, Alkema W, Fusetti F, Liu S, et al. Homer3 regulates the establishment of neutrophil polarity. Abstracts: AACR Special Conference: Tumor Immunology and Immunotherapy: A New Chapter. 2015; 3(10 Supplement):B48.
42. Esteban F, Ruiz-Cabello F, Concha A, Perez Ayala M, Delgado M, Garrido F. Relationship of 4F2 antigen with local growth and metastatic potential of squamous cell carcinoma of the larynx. Cancer. 1990; 66(7):1493-8.
43. Kaira K, Oriuchi N, Imai H, Shimizu K, Yanagitani N, Sunaga N, et al. 1-type amino acid transporter 1 and CD98 expression in primary and metastatic sites of human neoplasms. Cancer Sci. 2008; 99(12):2380-6.
44. Kobayashi K, Ohnishi A, Promsuk J, Shimizu S, Kanai Y, Shiokawa Y, et al. Enhanced tumor growth elicited by L-type amino acid transporter 1 in human malignant glioma cells. Neurosurgery. 2008; 62(2):493-503; discussion -4.
45. Fan L, Li A, Li W, Cai P, Yang B, Zhang M, et al. Novel role of Sarco/endoplasmic reticulum calcium ATPase 2 in development of colorectal cancer and its regulation by F36, a curcumin analog. Biomed Pharmacother. 2014; 68(8):1141-8.
46. Wang J W, Gamsby J J, Highfill S L, Mora L B, Bloom G C, Yeatman T J, et al. Deregulated expression of LRBA facilitates cancer cell growth. Oncogene. 2004; 23(23):4089-97.
47. Xu L, Mohammad K S, Wu H, Crean C, Poteat B, Cheng Y, et al. Cell Adhesion Molecule CD166 Drives Malignant Progression and Osteolytic Disease in Multiple Myeloma. Cancer research. 2016; 76(23):6901-10.
48. Wiiger M T, Gehrken H B, Fodstad O, Maelandsmo G M, Andersson Y. A novel human recombinant single-chain antibody targeting CD166/ALCAM inhibits cancer cell invasion in vitro and in vivo tumour growth. Cancer Immunol Immunother. 2010; 59(11):1665-74.
49. Xu J, Shen Z Y, Chen X G, Zhang Q, Bian H J, Zhu P, et al. A randomized controlled trial of Licartin for preventing hepatoma recurrence after liver transplantation. Hepatology. 2007; 45(2):269-76.
50. Abengozar M A, de Frutos S, Ferreiro S, Soriano J, Perez-Martinez M, Olmeda D, et al. Blocking ephrinB2 with highly specific antibodies inhibits angiogenesis, lymphangiogenesis, and tumor growth. Blood. 2012; 119(19):4565-76.
51. James M A. Targeting CLPTM1L for treatment and prevention of cancer. 2016(US patent 20160333083).
52. Puskas L G, Man I, Szebeni G, Tiszlavicz L, Tsai S, James M A. Novel Anti-CRR9/CLPTM1L Antibodies with Antitumorigenic Activity Inhibit Cell Surface Accumulation, PI3K Interaction, and Survival Signaling. Molecular cancer therapeutics. 2016; 15(5):985-97.
53. Bellucci R, Wu C J, Chiaretti S, Weller E, Davies F E, Alyea E P, et al. Complete response to donor lymphocyte infusion in multiple myeloma is associated with antibody responses to highly expressed antigens. Blood. 2004; 103(2):656-63.
54. Lex A, Gehlenborg N, Strobelt H, Vuillemot R, Pfister H. UpSet: Visualization of Intersecting Sets. IEEE Trans Vis Comput Graph. 2014; 20(12):1983-92.

### References for Supplemental Methods and FIGS. 9-12

1. Gundry R L, Raginski K, Tarasova Y, Tchernyshyov I, Bausch-Fluck D, Elliott S T, et al. The mouse C2C12 myoblast cell surface N-linked glycoproteome: identification, glycosite occupancy, and membrane orientation. Molecular & cellular proteomics: MCP. 2009; 8(11):2555-69.
2. Wollscheid B, Bausch-Fluck D, Henderson C, O'Brien R, Bibel M, Schiess R, et al. Mass-spectrometric identification and relative quantification of N-linked cell surface glycoproteins. Nature biotechnology. 2009; 27(4):378-86.
3. Gundry R L, Riordon D R, Tarasova Y, Chuppa S, Bhattacharya S, Juhasz O, et al. A cell surfaceome map for immunophenotyping and sorting pluripotent stem cells. Molecular & cellular proteomics: MCP. 2012; 11(8):303-16.
4. Waas M, Pereckas M, Jones Lipinski R A, Ashwood C, Gundry R L. SP2: Rapid and Automatable Contaminant Removal from Peptide Samples for Proteomic Analyses. J Proteome Res. 2019; 18(4):1644-56.
5. MacLean B, Tomazela D M, Shulman N, Chambers M, Finney G L, Frewen B, et al. Skyline: an open source document editor for creating and analyzing targeted proteomics experiments. Bioinformatics. 2010; 26(7):966-8.
6. Peterson A C, Russell J D, Bailey D J, Westphall M S, Coon J J. Parallel reaction monitoring for high resolution and high mass accuracy quantitative, targeted proteomics. Molecular & cellular proteomics: MCP. 2012; 11(11):1475-88.
7. Hajek R, Okubote S A, Svachova H. Myeloma stem cell concepts, heterogeneity and plasticity of multiple myeloma. British journal of haematology. 2013; 163(5):551-64.
8. Garfall A L, Maus M V, Hwang W T, Lacey S F, Mahnke Y D, Melenhorst J J, et al. Chimeric Antigen Receptor T Cells against CD19 for Multiple Myeloma. The New England journal of medicine. 2015; 373(11):1040-7.
9. Robillard N, Avet-Loiseau H, Garand R, Moreau P, Pineau D, Rapp M J, et al. CD20 is associated with a small mature plasma cell morphology and t(11;14) in multiple myeloma. Blood. 2003; 102(3):1070-1.
10. Robillard N, Jego G, Pellat-Deceunynck C, Pineau D, Puthier D, Mellerin M P, et al. CD28, a marker associated with tumoral expansion in multiple myeloma. Clinical cancer research: an official journal of the American Association for Cancer Research. 1998; 4(6):1521-6.
11. Asosingh K, Gunthert U, Bakkus M H, De Raeve H, Goes E, Van Riet I, et al. In vivo induction of insulin-like growth factor-I receptor and CD44v6 confers homing and adhesion to murine multiple myeloma cells. Cancer research. 2000; 60(11):3096-104.
12. Van Driel M, Gunthert U, van Kessel A C, Joling P, Stauder R, Lokhorst H M, et al. CD44 variant isoforms are involved in plasma cell adhesion to bone marrow stromal cells. Leukemia. 2002; 16(1):135-43.
13. Liebisch P, Eppinger S, Schopflin C, Stehle G, Munzert G, Dohner H, et al. CD44v6, a target for novel antibody treatment approaches, is frequently expressed in multiple myeloma and associated with deletion of chromosome arm 13q. Haematologica. 2005; 90(4):489-93.
14. Kumar S, Rajkumar S V, Kimlinger T, Greipp P R, Witzig T E. CD45 expression by bone marrow plasma cells in multiple myeloma: clinical and biological correlations. Leukemia. 2005; 19(8):1466-70.
15. Morice W G, Hanson C A, Kumar S, Frederick L A, Lesnick C E, Greipp P R. Novel multi-parameter flow cytometry sensitively detects phenotypically distinct plasma cell subsets in plasma cell proliferative disorders. Leukemia. 2007; 21(9):2043-6.
16. Asosingh K, De Raeve H, Van Riet I, Van Camp B, Vanderkerken K. Multiple myeloma tumor progression in the 5T2MM murine model is a multistage and dynamic process of differentiation, proliferation, invasion, and apoptosis. Blood. 2003; 101(8):3136-41.
17. Pellat-Deceunynck C, Bataille R. Normal and malignant human plasma cells: proliferation, differentiation, and expansions in relation to CD45 expression. Blood Cells Mol Dis. 2004; 32(2):293-301.
18. Bataille R, Robillard N, Pellat-Deceunynck C, Amiot M. A cellular model for myeloma cell growth and maturation based on an intraclonal CD45 hierarchy. Immunological reviews. 2003; 194:105-11.
19. Sampaio M S, Vettore A L, Yamamoto M, Chauffaille Mde L, Zago M A, Colleoni G W. Expression of eight genes of nuclear factor-kappa B pathway in multiple myeloma using bone marrow aspirates obtained at diagnosis. Histol Histopathol. 2009; 24(8):991-7.
20. Schmidmaier R, Morsdorf K, Baumann P, Emmerich B, Meinhardt G. Evidence for cell adhesion-mediated drug resistance of multiple myeloma cells in vivo. Int J Biol Markers. 2006; 21(4):218-22.
21. Zheng Y, Yang J, Qian J, Qiu P, Hanabuchi S, Lu Y, et al. PSGL-1/selectin and ICAM-1/CD18 interactions are involved in macrophage-induced drug resistance in myeloma. Leukemia. 2013; 27(3):702-10.
22. Wichert S, Juliusson G, Johansson A, Sonesson E, Teige I, Wickenberg A T, et al. A single-arm, open-label, phase 2 clinical trial evaluating disease response following treatment with BI-505, a human anti-intercellular adhesion molecule-1 monoclonal antibody, in patients with smoldering multiple myeloma. PLoS One. 2017; 12(2):e0171205.
23. Drach J, Gattringer C, Huber H. Expression of the neural cell adhesion molecule (CD56) by human myeloma cells. Clin Exp Immunol. 1991; 83(3):418-22.
24. Leo R, Boeker M, Peest D, Hein R, Bartl R, Gessner J E, et al. Multiparameter analyses of normal and malignant human plasma cells: CD38++, CD56+, CD54+, cIg+ is the common phenotype of myeloma cells. Annals of hematology. 1992; 64(3):132-9.
25. Kraj M, Sokolowska U, Kopec-Szlezak J, Poglod R, Kruk B, Wozniak J, et al. Clinicopathological correlates of plasma cell CD56 (NCAM) expression in multiple myeloma. Leukemia & lymphoma. 2008; 49(2):298-305.
26. Tai Y T, Mayes P A, Acharya C, Zhong M Y, Cea M, Cagnetta A, et al. Novel anti-B-cell maturation antigen antibody-drug conjugate (GSK2857916) selectively induces killing of multiple myeloma. Blood. 2014; 123(20):3128-38.
27. Carpenter R O, Evbuomwan M O, Pittaluga S, Rose J J, Raffeld M, Yang S, et al. B-cell maturation antigen is a promising target for adoptive T-cell therapy of multiple myeloma. Clinical cancer research: an official journal of the American Association for Cancer Research. 2013; 19(8):2048-60.
28. Sukowati C H, Anfuso B, Torre G, Francalanci P, Croce L S, Tiribelli C. The expression of CD90/Thy-1 in hepatocellular carcinoma: an in vivo and in vitro study. PLoS One. 2013; 8(10):e76830.
29. Yang Z F, Ho D W, Ng M N, Lau C K, Yu W C, Ngai P, et al. Significance of CD90+ cancer stem cells in human liver cancer. Cancer Cell. 2008; 13(2):153-66.
30. Bahnassy A A, Fawzy M, El-Wakil M, Zekri A R, Abdel-Sayed A, Sheta M. Aberrant expression of cancer stem cell markers (CD44, CD90, and CD133) contributes to disease progression and reduced survival in hepatoblastoma patients: 4-year survival data. Transl Res. 2015; 165(3):396-406.
31. Chen J F, Zhang L J, Zhao A L, Wang Y, Wu N, Xiong H C, et al. Abnormal expression of Thy-1 as a novel tumor marker in lung cancer and its prognostic significance. Zhonghua Yi Xue Za Zhi. 2005; 85(27):1921-5.
32. Yan X, Luo H, Zhou X, Zhu B, Wang Y, Bian X. Identification of CD90 as a marker for lung cancer stem cells in A549 and H446 cell lines. Oncol Rep. 2013; 30(6):2733-40.
33. Shi J L, Fu L, Ang Q, Wang G J, Zhu J, Wang W D. Overexpression of ATP1B1 predicts an adverse prognosis in cytogenetically normal acute myeloid leukemia. Oncotarget. 2016; 7(3):2585-95.
34. Yamaguchi M, Ohno T, Oka K, Taniguchi M, Ito M, Kita K, et al. De novo CD5-positive diffuse large B-cell lymphoma: clinical characteristics and therapeutic outcome. British journal of haematology. 1999; 105(4):1133-9.
35. Soleimani A, Schmieg J J, Brown T C, Yin L, Safah H, Saba N S. CD5-Negative Mantle Cell Lymphoma Defines a Distinct Disease Entity Characterized By an Indolent Clinical Course Irrespective of Known Prognostic Markers. Blood. 2017; 130(Supplemental 1):4061.
36. Rietbergen M M, Martens-de Kemp S R, Bloemena E, Witte B I, Brink A, Baatenburg de Jong R J, et al. Cancer stem cell enrichment marker CD98: a prognostic factor for survival in patients with human papillomavirus-positive oropharyngeal cancer. Eur J Cancer. 2014; 50(4):765-73.
37. Satoh T, Kaira K, Takahashi K, Takahashi N, Kanai Y, Asao T, et al. Prognostic Significance of the Expression of CD98 (4F2hc) in Gastric Cancer. Anticancer Res. 2017; 37(2):631-6.
38. Toyoda M, Kaira K, Shino M, Sakakura K, Takahashi K, Takayasu Y, et al. CD98 as a novel prognostic indicator for patients with stage III/IV hypopharyngeal squamous cell carcinoma. Head Neck. 2015; 37(11):1569-74.
39. Sasaki Y, Tamura M, Takeda K, Ogi K, Nakagaki T, Koyama R, et al. Identification and characterization of the intercellular adhesion molecule-2 gene as a novel p53 target. Oncotarget. 2016; 7(38):61426-37.
40. Hiraoka N, Yamazaki-Itoh R, Ino Y, Mizuguchi Y, Yamada T, Hirohashi S, et al. CXCL17 and ICAM2 are associated with a potential anti-tumor immune response in early intraepithelial stages of human pancreatic carcinogenesis. Gastroenterology. 2011; 140(1):310-21.
41. Fan L, Li A, Li W, Cai P, Yang B, Zhang M, et al. Novel role of Sarco/endoplasmic reticulum calcium ATPase 2 in development of colorectal cancer and its regulation by F36, a curcumin analog. Biomed Pharmacother. 2014; 68(8):1141-8.
42. Hu Y, Wang L, Wang L, Wu X, Wu X, Gu Y, et al. Preferential cytotoxicity of bortezomib toward highly malignant human liposarcoma cells via suppression of MDR1 expression and function. Toxicol Appl Pharmacol. 2015; 283(1):1-8.
43. Johnson S A S, Lin J J, Walkey C J, Leathers M P, Coarfa C, Johnson D L. Elevated TATA-binding protein expression drives vascular endothelial growth factor expression in colon cancer. Oncotarget. 2017; 8(30):48832-45.
44. Andres S A, Brock G N, Wittliff J L. Interrogating differences in expression of targeted gene sets to predict breast cancer outcome. BMC Cancer. 2013; 13:326.
45. Husa A M, Magic Z, Larsson M, Fornander T, Perez-Tenorio G. EPH/ephrin profile and EPHB2 expression predicts patient survival in breast cancer. Oncotarget. 2016; 7(16):21362-80.
46. Alam S M, Fujimoto J, Jahan I, Sato E, Tamaya T. Overexpression of ephrinB2 and EphB4 in tumor advancement of uterine endometrial cancers. Ann Oncol. 2007; 18(3):485-90.
47. Oweida A, Bhatia S, Hirsch K, Calame D, Griego A, Keysar S, et al. Ephrin-B2 overexpression predicts for poor prognosis and response to therapy in solid tumors. Mol Carcinog. 2017; 56(3):1189-96.
48. Tachibana M, Tonomoto Y, Hyakudomi R, Hyakudomi M, Hattori S, Ueda S, et al. Expression and prognostic significance of EFNB2 and EphB4 genes in patients with oesophageal squamous cell carcinoma. Dig Liver Dis. 2007; 39(8):725-32.
49. Shen T Y, Mei L L, Qiu Y T, Shi Z Z. Identification of candidate target genes of genomic aberrations in esophageal squamous cell carcinoma. Oncol Lett. 2016; 12(4):2956-61.
50. Aghaei M, Karami-Tehrani F, Salami S, Atri M. Adenosine deaminase activity in the serum and malignant tumors of breast cancer: the assessment of isoenzyme ADA1 and ADA2 activities. Clin Biochem. 2005; 38(10):887-91.
51. Ni Z, Chen Q, Lai Y, Wang Z, Sun L, Luo X, et al. Prognostic significance of CLPTM1L expression and its effects on migration and invasion of human lung cancer cells. Cancer Biomark. 2016; 16(3):445-52.
52. James M A, Wen W, Wang Y, Byers L A, Heymach J V, Coombes K R, et al. Functional characterization of CLPTM1L as a lung cancer risk candidate gene in the 5p15.33 locus. PLoS One. 2012; 7(6):e36116.
53. Xu L, Mohammad K S, Wu H, Crean C, Poteat B, Cheng Y, et al. Cell Adhesion Molecule CD166 Drives Malignant Progression and Osteolytic Disease in Multiple Myeloma. Cancer research. 2016; 76(23):6901-10.
54. Donizy P, Zietek M, Halon A, Leskiewicz M, Kozyra C, Matkowski R. Prognostic significance of ALCAM (CD166/MEMD) expression in cutaneous melanoma patients. Diagn Pathol. 2015; 10:86.
55. Bausch-Fluck D, Hofmann A, Bock T, Frei A P, Cerciello F, Jacobs A, et al. A mass spectrometric-derived cell surface protein atlas. PLoS One. 2015; 10(3):e0121314.

We claim:

1. A method of detecting multiple myeloma in a sample from a subject having or suspected of having multiple myeloma, the method comprising:
detecting the expression of one or more proteins listed in Table 3 at a higher level in the sample than in a non-cancer control.

2. The method of claim 1, wherein the method comprises detecting three or more proteins listed in Table 3.

3. The method claim 2, wherein the method comprises detecting five or more proteins listed in Table 3.

4. The method of claim 3, wherein the method comprises detecting nine or more proteins listed in Table 3.

5. The method of claim 1, wherein the one or more proteins comprise at least one of the following: CD5, CD166, CD147, CD98h, CD205, LRBA, CLPTM1L, Homer3, EFNB2, and combinations thereof.

6. The method of claim 5, wherein the one or more proteins comprise at least one of the following: LRBA, CLPTM1L, Homer3, EFNB2, and combinations thereof.

7. The method of claim 5, wherein the one or more proteins comprise at least one of the following: CD5, CD166, CD147, CD98hc, CD205, and combinations thereof.

8. The method of claim 1, wherein the method further comprises treating the subject with an anti-cancer therapy.

9. The method of claim 8, wherein the anti-cancer therapy specifically targets the protein that was detected.

10. A method of treating multiple myeloma, the method comprising:
detecting the expression of one or more proteins in a sample from a subject having or suspected of having multiple myeloma, wherein the one or more proteins are selected from the group consisting of CD5, CD166, CD147, CD98h, CD205, LRBA, CLPTM1L, Homer3, EFNB2, and combinations thereof, and
treating the subject with an anti-cancer therapy if at least one of the one or more proteins are detected at a higher level in the sample than in a non-cancer control.

11. The method of claim 10, wherein the method comprises one or more of the following:
(a) detecting increased expression of CD5 in the sample, and treating the subject with an anti-CD5 anti-cancer therapy, preferably a CD5 chimeric antigen receptor T cell;
(b) detecting increased expression of CD147 in the sample, and treating the subject with an anti-CD147 anti-cancer therapy, preferably a radioimmunotherapy;
(c) detecting increased expression of CD205 in the sample, and treating the subject with an anti-CD205 anti-cancer therapy, preferably an anti-CD205 antibody-drug conjugate;
(d) detecting increased expression of CD98 in the sample, and treating the subject with an anti-CD98 anti-cancer therapy, preferably an antibody against CD98; and/or
(e) detecting increased expression of CD166 in the sample, and treating the subject with an anti-CD166 anti-cancer therapy, preferably an antibody-drug conjugate targeting CD166.

12. The method of claim 10, wherein the one or more proteins comprise at least one of the following: LRBA, CLPTM1L, Homer3, EFNB2, and combinations thereof.

13. The method of claim 10, wherein the one or more proteins comprise at least one of the following: CD5, CD166, CD147, CD98hc, CD205, and combinations thereof.

14. The method of claim 1, wherein the one or more proteins are detected using a mass spectrometry-based method selected from cell surface capture (CSC), and a parallel reaction monitoring (PRM) assay.

15.-16. (canceled)

17. The method of claim 1, wherein the one or more proteins are detected using flow cytometry.

18. The method of claim 1, wherein the sample is a biopsy from the subject.

19. The method of claim 1, wherein the sample is a bone marrow sample from the subject and the method further comprises obtaining a bone marrow sample from a subject.

20. The method of claim 1, wherein the method further comprises isolating CD138+ cells from the sample prior to detecting the one or more proteins.

21. (canceled)

22. A kit for detecting multiple myeloma in a sample from a subject having or suspected of having multiple myeloma, the kit comprising one or more antibodies that are specific to one or more proteins listed in Table 3.

23. The kit of claim 22, wherein the kit further comprises one or more of:
(i) a solid surface to which the one or more antibodies are attached;
(ii) a reagent for isolating mononuclear cells;
(iii) a reagent for isolating CD138+ cells;
(iv) one or more antibodies specific for a protein selected from CD5, CD166, CD147, CD98h, CD205, LRBA, CLPTM1L, Homer3, and EFNB2;
(v) one or more antibodies specific for a protein selected from CD138, CD38−, CD45, CD19 and CD56.

24.-29. (canceled)
